(19) United States
(12) Patent Application Publication
(10) Pub. No.: US 2017/0275302 A1
(43) Pub. Date: Sep. 28, 2017

(54) INHIBITORS OF THE RENAL OUTER MEDULLARY POTASSIUM CHANNEL

(71) Applicants: Alexander PASTERNAK, Kenilworth, NJ (US); Barbara PIO, Kenilworth, NJ (US); Harry R. CHOBANIAN, Kenilworth, NJ (US); Zhi-Cai SHI, Kenilworth, NJ (US); Shuzhi DONG, Kenilworth, NJ (US); Yan GUO, Kenilworth, NJ (US); Shawn P. WALSH, Kenilworth, NJ (US); Zhiqiang GUO, Rahway, NJ (US); Ronald D. FERGUSON, Rahway, NJ (US); Brian CATO, Secaucus, NJ (US); Merck Sharp & Dohme Corp., Rahway, NJ (US)

(72) Inventors: Alexander Pasternak, Princeton, NJ (US); Barbara Pio, West Orange, NJ (US); Harry Chobanian, Aberden, NJ (US); Zhi-Cai Shi, Monmouth Junction, NJ (US); Shuzhi Dong, Plainsboro, NJ (US); Yan Guo, Westfield, NJ (US); Shawn P. Walsh, Bridgewater, NJ (US); Zhiqiang Guo, Morganville, NJ (US); Ronald D. Ferguson II, Scotch Plains, NJ (US); Brian Cato, Secaucus, NJ (US)

(21) Appl. No.: 15/505,266

(22) PCT Filed: Oct. 9, 2015
(86) PCT No.: PCT/US15/54805

§ 371 Date: Feb. 21, 2017

Related U.S. Application Data
(60) Provisional application No. 62/063,664, filed on Oct. 14, 2014.

(51) Int. Cl. C07D 519/00; A61K 31/4985; C07D 471/10; A61K 31/435; C07D 498/10; A61K 31/519; A61K 31/437; A61K 31/4365; A61K 31/4375; A61K 31/4355; A61K 45/06; A61K 31/5025

(52) U.S. Cl. CPC C07D 519/00; A61K 45/06; A61K 31/4985; C07D 471/10; A61K 31/435; A61K 31/5025; A61K 31/519; A61K 31/437; A61K 31/4365; A61K 31/4375; A61K 31/4355; C07D 498/10

(57) ABSTRACT

The present invention provides compounds of Formula (I) and the pharmaceutically acceptable salts thereof, which are inhibitors of the ROMK (Kir1.1) channel. The compounds may be used as diuretic and/or natriuretic agents and for the therapy and prophylaxis of medical conditions including cardiovascular diseases such as hypertension, heart failure and chronic kidney disease and conditions associated with excessive salt and water retention.

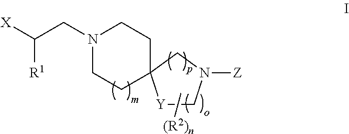

### BACKGROUND OF THE INVENTION

The Renal Outer Medullary Potassium (ROMK) channel Kir1.1) (see e.g., Ho, K., et al., Cloning and expression of an inwardly rectifying ATP-regulated potassium channel, Nature, 1993, 362(6415): p. 31-8.1, 2; and Shuck, M. E., et al., Cloning and characterization of multiple forms of the human kidney ROM-K potassium channel, J Biol Chem, 1994, 269(39): p. 24261-70) is a member of the inward rectifier family of potassium channels expressed in two regions of the kidney: thick ascending loop of Henle (TALH) and cortical collecting duct (CCD) (see Hebert, S. C., et al., Molecular diversity and regulation of renal potassium channels, Physiol Rev, 2005, 85(1): p. 319-713). At the TALH, ROMK participates in potassium recycling across the luminal membrane which is critical for the function of the Na⁺/K⁺/2Cl⁻ co-transporter, the rate-determining step for salt reuptake in this part of the nephron. At the CCD, ROMK provides a pathway for potassium secretion that is tightly coupled to sodium uptake through the amiloride-sensitive sodium channel (see Reinalter, S. C., et al., Pharmacotyping of hypokalaemic salt-losing tubular disorders, Acta Physiol Scand, 2004, 181(4): p. 513-21; and Wang, W., Renal potassium channels: recent developments, Curr Opin Nephrol Hypertens, 2004, 13(5): p. 549-55). Selective inhibitors of the ROMK channel (also referred to herein as inhibitors of ROMK or ROMK inhibitors) are expected to represent novel diuretics for the treatment of hypertension and other conditions where treatment with a diuretic would be beneficial with potentially reduced liabilities (i.e., hypo- or hyperkalemia, new onset of diabetes, dyslipidemia) over the currently used clinical agents (see Lifton, R. P., A. G. Gharavi, and D. S. Geller, Molecular mechanisms of human hypertension, Cell, 2001, 104(4): p. 545-56). Human genetics (Ji, W., et al., Rare independent mutations in renal salt handling genes contribute to blood pressure variation, Nat Genet, 2008, 40(5): p. 592-9; and Tobin, M. D., et al., Common variants in genes underlying monogenic hypertension and hypotension and blood pressure in the general population, Hypertension, 2008, 51(6): p. 1658-64) and genetic ablation of ROMK in rodents (see Lorenz, J. N., et al., Impaired renal NaCl absorption in mice lacking the ROMK potassium channel, a model for type II Bartter's syndrome, J Biol Chem, 2002, 277(40): p. 37871-80 and Lu, M., et al., Absence of small conductance K+channel (SK) activity in apical membranes of thick ascending limb and cortical collecting duct in ROMK (Bartter's) knockout mice, J Biol Chem, 2002, 277(40): p. 37881-7) support these expectations. To our knowledge, the first publicly disclosed small molecule selective inhibitors of ROMK, including VU590, were reported from work done at Vanderbilt University as described in Lewis, L. M., et al., High-Throughput Screening Reveals a Small-Molecule Inhibitor of the Renal Outer Medullary Potassium Channel and Kir7.1, Mol Pharmacol, 2009, 76(5): p. 1094-1103. The compound VU591 was later reported in Bhave, G. et al., Development of a Selective Small-Molecule Inhibitor of Kir1.1, the Renal Outer Medullary Potassium Channel, Mol Pharmacol, 2011, 79(1), p. 42-50, the text of which states that “ROMK (Kir1.1), is a putative drug target for a novel class of loop diuretics that would lower blood pressure without causing hypokalemia.”

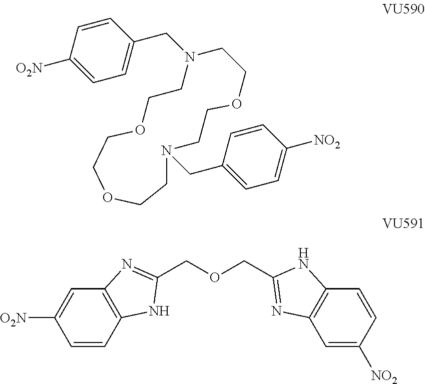 [SMILES: O=[N+]([O-])c1ccc(CN2CCOCCOCCN(Cc3ccc([N+](=O)[O-])cc3)CCOCC2)cc1 ; O=[N+]([O-])c1ccc2[nH]c(COCc3nc4cc([N+](=O)[O-])ccc4[nH]3)nc2c1]

Patent application publication number WO2010/129379, published Nov. 11, 2010 having common representative Merck Sharp & Dohme Corp., (also published as US2010/0286123 on same date), describes ROMK inhibitors having the generic formula:

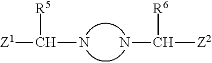

and, e.g., an embodiment

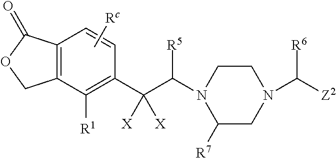

wherein R⁵ and R⁶ are independently —H, —C₁₋₆ alkyl, —C₃₋₆ cycloalkyl, —CF₃, —CHF₂, —CH₂F or —CH₂OH; X is —H, —OH, —OC₁₋₃alkyl, —F, oxo, NH₂ or —CH₃; and X¹ is —H or —CH₃.

Patent application publication number WO2012/058134, published May 3, 2012, having common representative Merck Sharp & Dohme Corp., describes ROMK inhibitors having the generic formula:

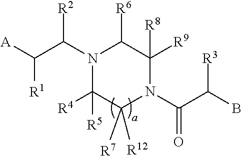

wherein A and B are mono and/or bicyclic aromatic groups; R² is —H,
—C₁₋₆ alkyl, —C₃₋₆ cycloalkyl, CF₃, —CH₂OH, or —CO₂R, or R² can be joined to R¹ or R^{10a} to form a ring; R³ is —H, —C₁₋₆ alkyl, —C₃₋₆ cycloalkyl, —OH, —F, —OC₁₋₃ alkyl, or —CH₂OH, or R³ can be joined to R^{10a} to form a ring.

Patent application publication number WO2012/058116, published May 3, 2012, having common representative Merck Sharp & Dohme Corp., describes ROMK inhibitors having the generic formula:

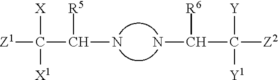

and, e.g., an embodiment

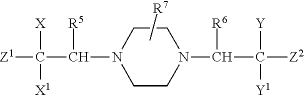

wherein R⁵ and R⁶ are independently —H, —C₁₋₆ alkyl or —C(O)OC₁₋₃alkyl; and X, X¹, Y and Y¹ are independently —H or —C₁₋₆alkyl; or Y¹ can be joined together with Z² to form a fused ring system. Additional published patent applications to Merck Sharp and Dohme, which describe ROMK inhibitors, include: WO2013/028474; WO2013/039802; WO2013/062892; WO2013/066714; WO2013/066717; WO2013/066718; and WO2013/090271. Other publications that disclose ROMK inhibitors and suggest that these compounds could be useful in the treatment of hypertension are: H. Tang et al., Discovery of Selective Small Molecule ROMK Inhibitors as Potential New Mechanism Diuretics, ACS Med. Chem. Lett. 2013, 3, p. 367-372; H. Tang, et al., Discovery of a Novel Sub-class of ROMK Channel Inhibitors Typified by 5-(2-(4-(2-(4-(1H-Tetrazol-1l-yl)phenyl)acetyl)piperazin-1-yl)ethyl) isobenzofuran-1 (3H)-one, Bioorg. Med. Chem. Lett. 2013, 23, pp. 5829-5823;

However, continuing discovery of selective small molecule inhibitors of ROMK is still needed for the development of new treatments for hypertension, heart failure, edematous states and related disorders. The compounds of Formula I and salts thereof of this invention are selective inhibitors of the ROMK channel and could be used for the treatment of hypertension, heart failure and other conditions where treatment with a diuretic or natriuretic would be beneficial.

### SUMMARY OF THE INVENTION

The present invention provides for compounds of the formula:

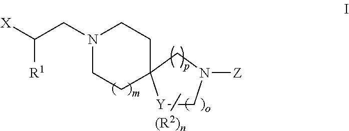

or a pharmaceutically acceptable salt thereof,

wherein:

X is

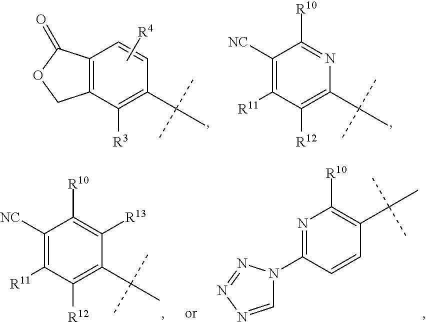

Y is —O— or —CH₂—;

Z is a N-containing multicyclic heteroaromatic group which is optionally substituted by one R⁶ group, or Z is a group of the formula:

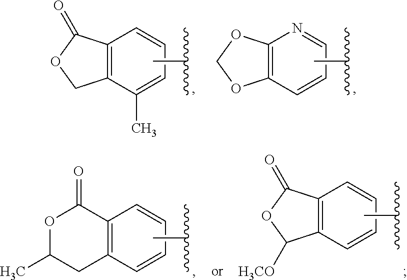

R is H, C₁₋₂ alkyl optionally substituted with 1-3 halogens, or —C(O)R⁵;

R¹ is —OR or halogen;

R² is OXO Or C₁₋₂ alkyl optionally substituted with 1-3 F;

R³ is H or CH₃;

R⁴ is H or CH₃;

R⁵ is CH₃ or C₃₋₆cycloalkyl;

R⁶ is halogen, —CN, C₃₋₆ cycloalkyl, furanyl, —SO₂N(R⁸)(R⁹), —OC₁₋₂ alkyl which is optionally substituted with 1-5 halogens, or C₁₋₂ alkyl which is optionally substituted with —SR⁷ or 1-5 halogens;

R⁷ is allyl or C₁₋₂ alkyl;

R⁸ is H or CH₃;

R⁹ is H or CH₃;

R¹⁰ is H, C₁₋₂ alkyl, or —OCH₃;

R¹¹ is H, C₁₋₂ alkyl, or —OCH₃;

R¹² is H, C₁₋₂ alkyl or —OCH₃;

R¹³ is H, halogen, C₁₋₂ alkyl or —OCH₃;

R¹⁴ is H, halogen, C₁₋₂ alkyl or —OCH₃;

R¹⁵ is H, halogen, C₁₋₂ alkyl or —OCH₃;

R¹⁶ is H, halogen, C₁₋₂ alkyl or —OCH₃;

m is 0 or 1;

n is 0 or 1;

o is 0, 1 or 2; and

p is 1, 2, or 3;

provided that o+p=2 or 3.

The compounds of Formula I are inhibitors of the ROMK (Kir1.1) channel. As a result, the compounds of Formula I could be used in methods of treatment, inhibition or amelioration of one or more disease states that could benefit from inhibition of ROMK. The compounds of this invention could be used in methods of treatment which comprise administering a therapeutically or prophylactically effective amount of a compound of Formula I to a patient in need of a diuretic and/or natriuretic agent. Therefore, the compounds of Formula I could be valuable pharmaceutically active compounds for the therapy, prophylaxis or both of medical conditions, including, but not limited to, cardiovascular diseases such as hypertension and heart failure as well as chronic kidney disease, and conditions associated with excessive salt and water retention. The compounds of this invention could further be used in combination with other therapeutically effective agents, including but not limited to, other drugs which are useful for the treatment of hypertension, heart failure and conditions associated with excessive salt and water retention. The invention furthermore relates to processes for preparing compounds of Formula I, and pharmaceutical compositions which comprise compounds of Formula I. These and other aspects of the invention will be evident from the description contained herein.

### DETAILED DESCRIPTION OF THE INVENTION

Embodiments of this invention comprise compounds of Formula I or pharmaceutically acceptable salts thereof.

Another embodiment of this invention is a compound of Formula I wherein the R⁶-substituted N-containing multicyclic heteroaromatic group is:

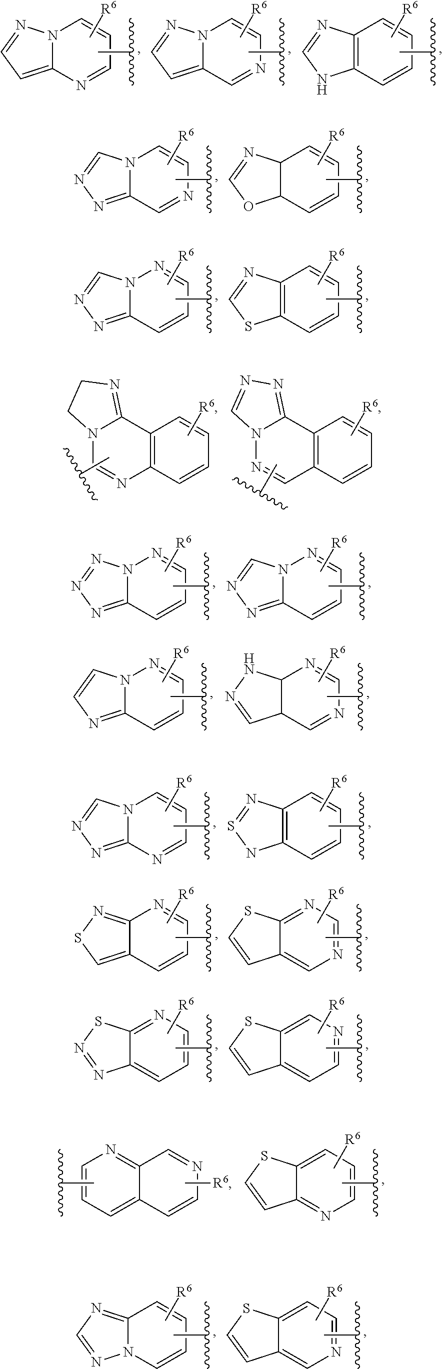

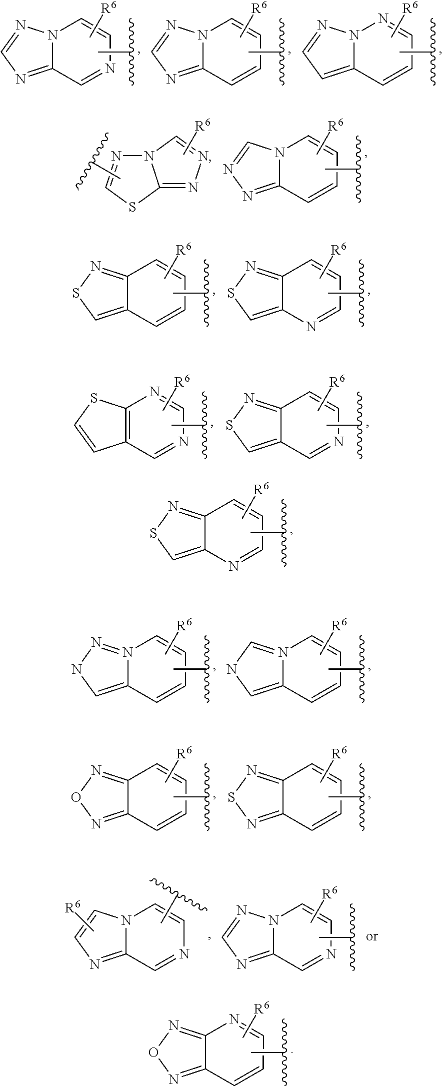

There are many embodiments of the structural elements of the compounds of this invention, as defined below. In general, structural elements for each substituent group can be independently substituted for one another.

In many embodiments of the compounds of this invention, R is H, —CH₃, or —C(═O)cyclopropyl.

In many embodiments, R¹ is —OH, —OCH₃, F, —OC(═O)cyclopropyl, or —OC(═O)CH₃.

In many embodiments, R² is oxo.

In many embodiments, R³ is CH₃.

In many embodiments, R⁴ is H.

In many embodiments, R⁵ is cyclopropyl or CH₃.

In many embodiments, R⁶ is H, F, —SO₂NH₂, —CH₂SCH₃, CF₃, CH₃, C₂H₅, —OCH₃, CN, cyclopropyl, or furanyl.

In many embodiments, R⁷ is allyl or —CH₃.

In many embodiments, R⁷ is —CH₃.

In many embodiments, R⁸ is H.

In many embodiments, R⁹ is H.

In many embodiments, R¹⁰ is H or CH₃.

In many embodiments, R¹¹ is H, CH₃ or —OCH₃.

In many embodiments, R¹¹ is H or —OCH₃.

In many embodiments, R¹² is H or CH₃.

In many embodiments, R¹³ is H, CH₃, or F.

In many embodiments, R¹⁴ is H, —OCH₃ or F.

In many embodiments, R¹⁵ is H, —OCH₃ or F.

In many embodiments, R¹⁶ is H, CH₃, F or Cl.

Another embodiment of this invention is a compound of Formula I having the structural formula II:

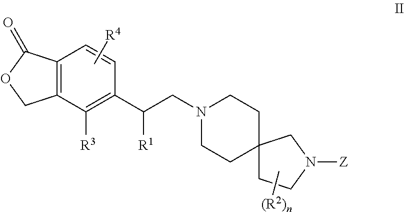

or a pharmaceutically acceptable salt thereof

wherein R¹, R³, R⁴, Z and n are as defined in Formula I.

Another embodiment of this invention is a compound of Formula IIa, which has the structural formula:

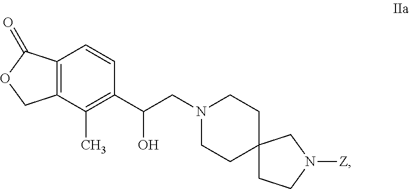

or a pharmaceutically acceptable salt thereof,

wherein:

Z is:

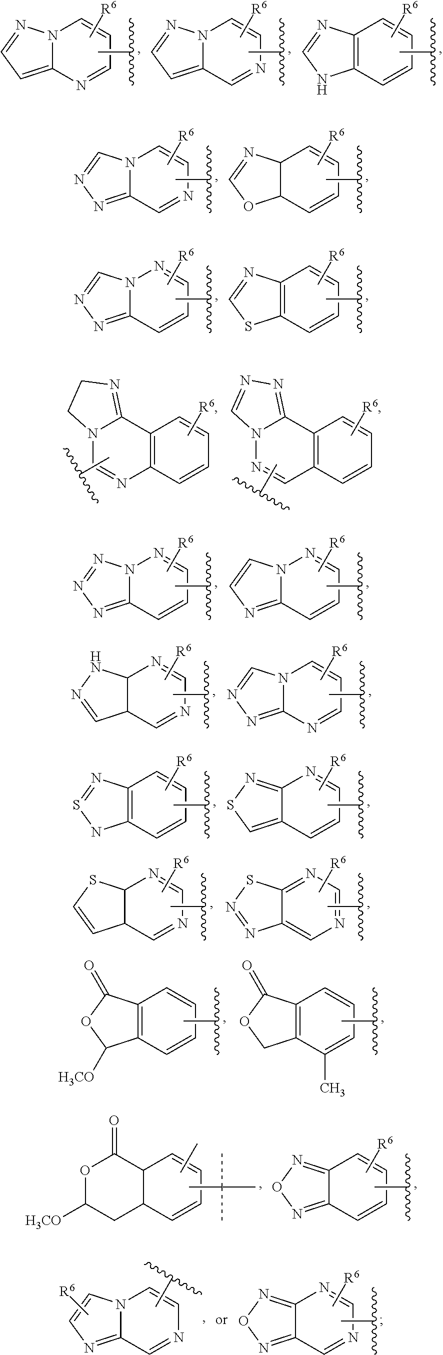

and R⁶ is as defined in Formula I.

Another embodiment of this invention is a compound of Formula IIb, which has the structural formula:

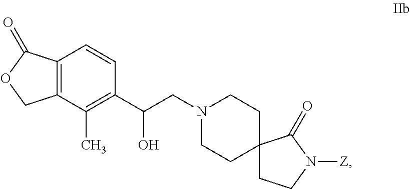

or a pharmaceutically acceptable salt thereof,

wherein:

Z is

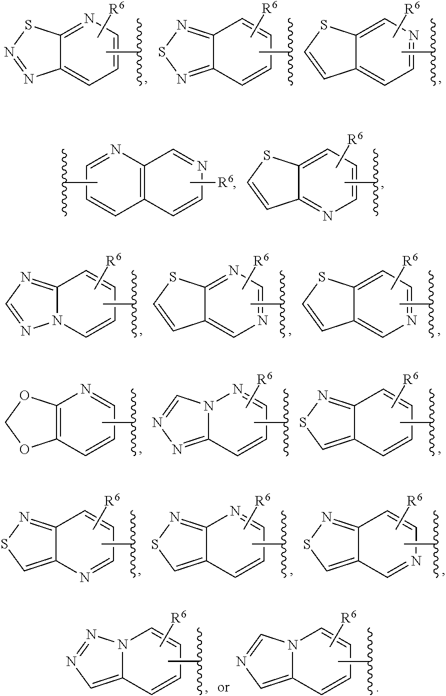

Another embodiment of this invention is a compound of Formula IIc, which has the structural formula:

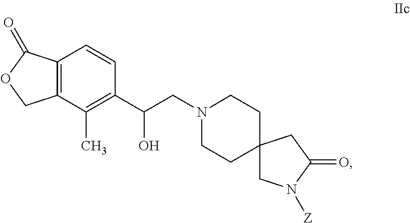

or a pharmaceutically acceptable salt thereof,

wherein:

Z is

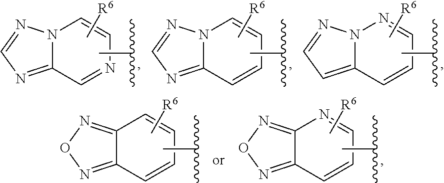

and R⁶ is as defined in Formula I.

Another embodiment of the present invention is a compound of Formula III, which has the structural formula:

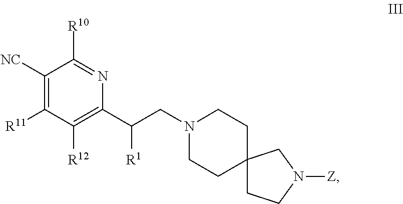

or a pharmaceutically acceptable salt thereof,

wherein:

Z is

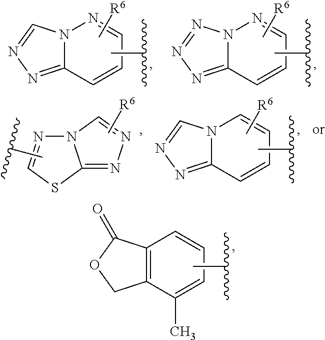

and R⁶ is as defined in Formula I.

Another embodiment of the present invention is a compound of Formula IV or IVa, which has the structural formula:

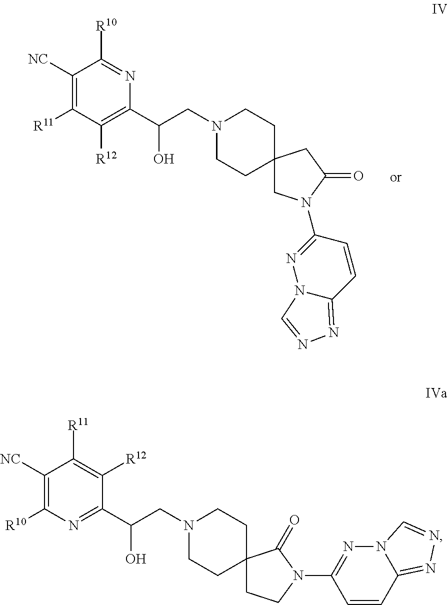

or a pharmaceutically acceptable salt thereof.

Another embodiment of the present invention is a compound of Formula V, which has the structural formula:

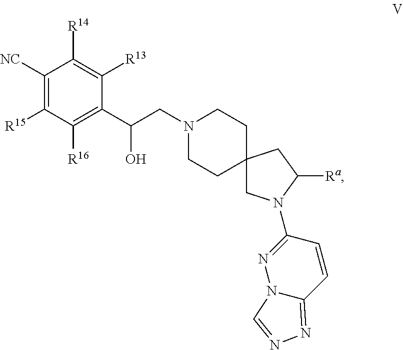

or a pharmaceutically acceptable salt thereof, wherein:

R^{a} is H or oxo;

R¹³ is H, halogen, C₁₋₂ alkyl, or —OC₁₋₂ alkyl;

R¹⁴ is H, halogen, C₁₋₂ alkyl, or —OC₁₋₂ alkyl;

R¹⁵ is H, halogen, C₁₋₂ alkyl, or —OC₁₋₂ alkyl; and

R¹⁶ is H, halogen, C₁₋₂ alkyl, or —OC₁₋₂ alkyl.

Another embodiment of the present invention is a compound of Formula VI, which has the structural formula:

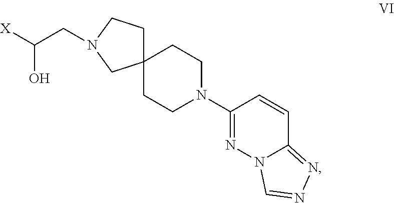

or a pharmaceutically acceptable salt thereof, wherein:

X is:

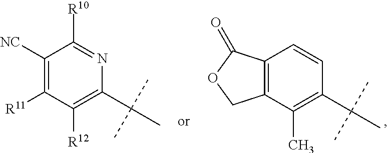

R¹⁰ is H or C₁₋₂ alkyl;

R¹ is H, C₁₋₂ alkyl, or —OC₁₋₂ alkyl; and

R¹² is H, C₁₋₂ alkyl, or —OC₁₋₂ alkyl.

Another embodiment of the present invention is a compound of Formula VII, which has the structural formula:

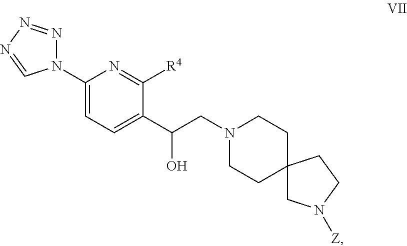

or a pharmaceutically acceptable salt thereof,

wherein:

Z is

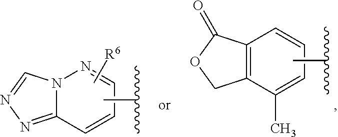

and R⁴ and R⁶ are as defined in Formula I.

Another embodiment of the present invention is a compound of Formula VIII, which has the structural formula:

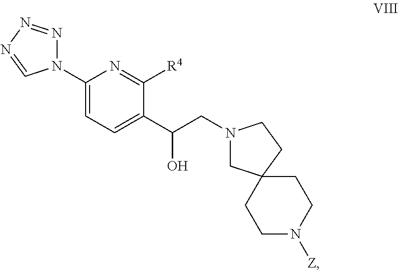

or a pharmaceutically acceptable salt thereof,

wherein:

Z is

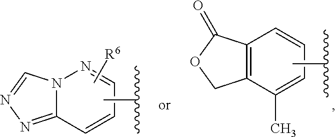

and R⁴ and R⁶ are as defined in Formula I.

Another embodiment of the present invention is a compound of Formula IX, which has the structural formula:

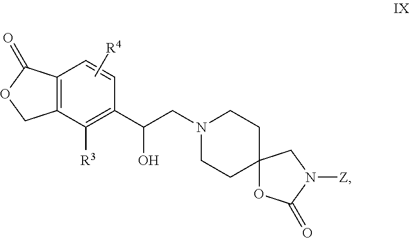

or a pharmaceutically acceptable salt thereof,

wherein

Z is

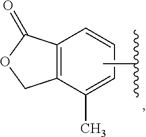

and R³ and R⁴ are as defined in Formula I.

Another embodiment of the present invention is a compound of Formula X or XI, which has the structural formula:

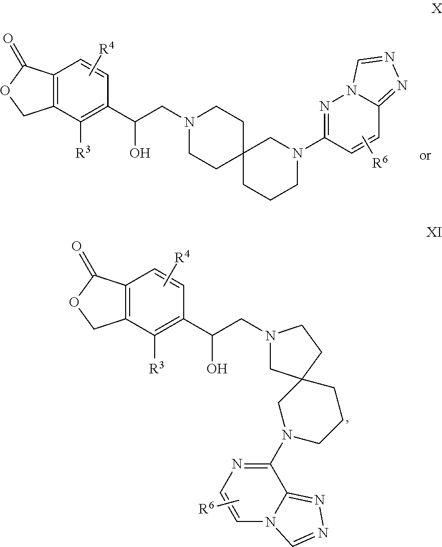

or a pharmaceutically acceptable salt thereof,

wherein R³, R⁴ and R⁶ are as defined in Formula I.

Another embodiment of the present invention is a compound represented by Formula I, IIa, IIb, IIc, III, VII, VIII, X or XI or a pharmaceutically acceptable salt thereof wherein R⁶ is H, —CN, halo, C₁₋₂ alkyl, C₁₋₂ alkyl-S-allyl, C₃₋₆ cycloalkyl, furanyl, —SO₂NH₂, or C₁₋₂ haloalkyl, wherein C₁₋₂ haloalkyl is substituted with 1-5 halogens.

Another embodiment of the present invention is a compound represented by Formula I, IIa, IIb, IIc, III, VII, VIII, X or XI or a pharmaceutically acceptable salt thereof wherein R⁶ is H.

Another embodiment of the present invention is a compound represented by Formula I which is selected from any of Examples 1-95, or a pharmaceutically acceptable salt thereof.

Another embodiment of the present invention is a compound represented by Formula I which is:
(R)-5-(2-(2-([1,2,4]triazolo[4,3-b]pyridazin-6-yl)-2,8-diazaspiro[4.5]decan-8-yl)-1-hydroxyethyl)-4-methylisobenzofuran-1 (3H)-one; (Ex. 5)
(R)-5-(1-hydroxy-2-(2-(tetrazolo[1,5-b]pyridazin-6-yl)-2,8-diazaspiro[4.5]decan-8-yl)ethyl)-4-methylisobenzofuran-1(3H)-one; (Ex 10);
(R)-2-([1,3]dioxolo[4,5-b]pyridin-7-yl)-8-(2-hydroxy-2-(4-methyl-1-oxo-1,3-dihydroisobenzofuran-5-yl)ethyl)-2,8-diazaspiro[4.5]decan-1-one; (Ex 38)
6-(2-(2-([1,2,4]triazolo[4,3-b]pyridazin-6-yl)-2,8-diazaspiro[4.5]decan-8-yl)-1-hydroxyethyl)-2-methylnicotinonitrile; (Ex 46)
4-(2-(2-([1,2,4]triazolo[4,3-b]pyridazin-6-yl)-2,8-diazaspiro[4.5]decan-8-yl)-1-hydroxyethyl)-2,5-difluoro-3-methylbenzonitrile; (54)
6-(2-(8-([1,2,4]triazolo[4,3-b]pyridazin-6-yl)-2,8-diazaspiro[4.5]decan-2-yl)-1-hydroxyethyl)-5-methylnicotinonitrile; (Ex 65)
6-(2-(8-([1,2,4]triazolo[4,3-b]pyridazin-6-yl)-2,8-diazaspiro[4.5]decan-2-yl)-1-hydroxyethyl)-2-methylnicotinonitrile; (Ex 67)
(R)-5-(2-(8-([1,2,4]triazolo[4,3-b]pyridazin-6-yl)-2,8-diazaspiro[4.5]decan-2-yl)-1-hydroxyethyl)-4-methylisobenzofuran-1(3H)-one; (Ex 70)
(R)-6-(1-hydroxy-2-(2-(4-methyl-1-oxo-1,3-dihydroisobenzofuran-5-yl)-2,8-diazaspiro[4.5]decan-8-yl)ethyl)-4-methoxynicotinonitrile; (Ex. 77)
(R)-5-(1-hydroxy-2-(2-(4-methyl-1-oxo-1,3-dihydroisobenzofuran-5-yl)-2,8-diazaspiro[4.5]decan-8-yl)ethyl)-4-methylisobenzofuran-1(3H)-one; (Ex 79)
(R)-8-(2-hydroxy-2-(4-methyl-1-oxo-1,3-dihydroisobenzofuran-5-yl)ethyl)-3-(4-methyl-1-oxo-1,3-dihydroisobenzofuran-5-yl)-1-oxa-3,8-diazaspiro[4.5]decan-2-one; (Ex. 81)
(R)-2-([1,2,3]triazolo[1,5-a]pyridin-5-yl)-8-(2-hydroxy-2-(4-methyl-1-oxo-1,3-dihydroisobenzofuran-5-yl)ethyl)-2,8-diazaspiro[4.5]decan-1-one; (Ex 88)
(R)-2-(benzo[c][1,2,5]oxadiazol-5-yl)-8-(2-hydroxy-2-(4-methyl-1-oxo-1,3-dihydroisobenzofuran-5-yl)ethyl)-2,8-diazaspiro[4.5]decan-3-one; (Ex 89);
(R)-5-(2-(2-([1,2,5]oxadiazolo[3,4-b]pyridin-6-yl)-2,8-diazaspiro[4.5]decan-8-yl)-1-hydroxyethyl)-4-methylisobenzofuran-1(3H)-one; (Ex 92)
(R)-5-(2-(2-([1,2,5]oxadiazolo[3,4-b]pyridin-5-yl)-2,8-diazaspiro[4.5]decan-8-yl)-1-hydroxyethyl)-4-methylisobenzofuran-1(3H)-one; (Ex 95)
   or a pharmaceutically acceptable salt thereof.

All structural formulae, embodiments and classes thereof described herein include the pharmaceutically acceptable salts of the compounds defined herein.

“Alkyl” is intended to include both branched- and straight-chain saturated aliphatic hydrocarbon groups having, e.g., 1-12, 1-6 or 1-4 carbon atoms. Commonly used abbreviations for alkyl groups are used throughout the specification. For example the term “C₁₋₆ alkyl” (or “C₁-C₆ alkyl”), means linear or branched chain alkyl groups, including all isomers, having the specified number of carbon atoms and includes all of the hexyl and pentyl isomers as well as n-, iso-, sec- and tert-butyl (butyl, s-butyl, i-butyl, t-butyl; Bu=butyl), n- and i-propyl (Pr=propyl), ethyl (Et) and methyl (Me).

“Alkoxy” is an alkyloxy group wherein the alkyl group is as previously defined and the bond to the parent moiety is through the oxy group. Non-limiting examples include —OCH₃, —OCH₂CH₃, etc.

“Halogen” means a fluorine, chlorine, bromine or iodine atom. “Halo” means —F, —Cl, —Br, or —I. A non-limiting examples includes fluorine or fluoro.

“Haloalkyl” means a halo-alkyl group in which the halo and alkyl groups are as previously defined. The bond to the parent moiety is through the alkyl group. Non-limiting examples include —CH₂CF₃ and —CF₃.

“Cycloalkyl” is a cyclized alkyl ring having 3-12 or 3-6 carbon atoms. Examples of cycloalkyl include cyclopropyl, cyclobutyl, cyclopentyl and cyclohexyl.

“Oxo” is a “C═(O)” functional group, that is a carbonyl group.

A “N-containing multicyclic heteroaromatic” group means a bicyclic or tricyclic fused ring system containing 9 to 14 ring members in which from 1 to 5 ring members are heteroatoms that are independently selected from the group consisting of nitrogen, sulfur or oxygen and the remainder of the ring members are carbon, provided that at least one of the ring members is nitrogen. The point of attachment to the parent moiety is through any available ring member. Further, no two adjacent ring members may be oxygen or sulfur. Non-limiting examples of N-containing heteroaromatic groups (showing the R⁶ substituent) include:

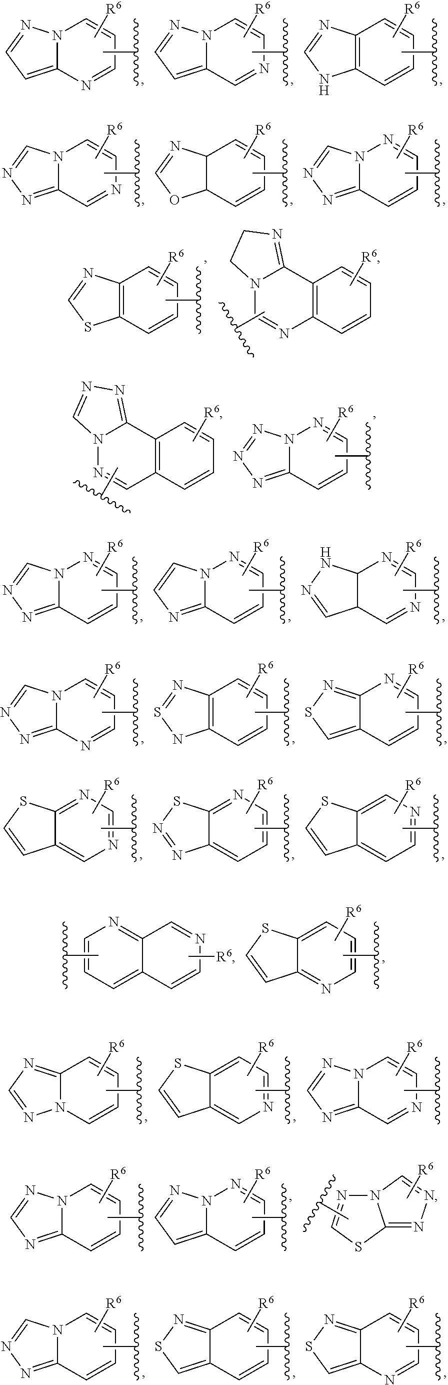

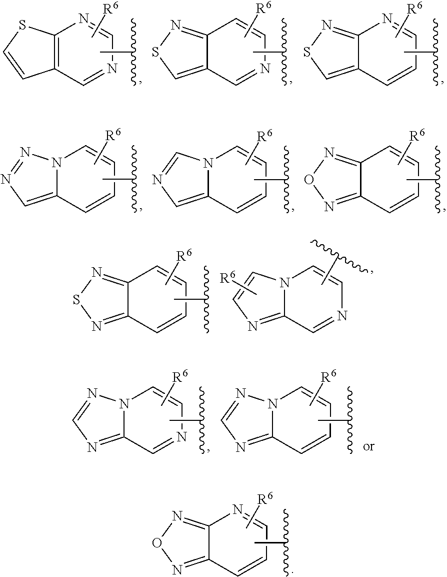

Unless expressly depicted or described otherwise, variables depicted in a structural formula with a “floating” bond, such as R⁶, are permitted on any available carbon atom in the ring to which the variable is attached. If the ring is multicyclic (e.g., a bicyclic ring), then the variable may be attached to any carbon in the multicyclic (e.g., bicyclic) ring.

The symbols
[symbol] or [symbol]
   refer to the rest of the molecule described by any of the formulae to which X or Z attaches.

In choosing compounds of the present invention, one of ordinary skill in the art will recognize that the various substituents, i.e. R¹, R², etc., are to be chosen in conformity with well-known principles of chemical structure connectivity and stability.

The term “substituted” shall be deemed to include multiple degrees of substitution by a named substituent. Where multiple substituent moieties are disclosed or claimed, the substituted compound can be independently substituted by one or more of the disclosed or claimed substituent moieties, singly or plurally. By independently substituted, it is meant that the (two or more) substituents can be the same or different.

Where a substituent or variable has multiple definitions, it is understood that the substituent or variable is defined as being selected from the group consisting of the indicated definitions.

The compounds of Formula I may have one or more chiral (asymmetric) centers. The present invention encompasses all stereoisomeric forms of the compounds of Formula I. Centers of asymmetry that are present in the compounds of Formula I can all independently of one another have (R) or (S) configuration. When bonds to a chiral carbon are depicted as straight lines in the structural Formulas of the invention, or when a compound name is recited without an (R) or (S) chiral designation for a chiral carbon, it is understood that both the (R) and (S) configurations of each such chiral carbon, and hence each enantiomer or diastereomer and mixtures thereof, are embraced within the Formula or by the name. The production of specific stereoisomers or mixtures thereof may be identified in the Examples where such stereoisomers or mixtures were obtained, but this in no way limits the inclusion of all stereoisomers and mixtures thereof from being within the scope of this invention.

The invention includes all possible enantiomers and diastereomers and mixtures of two or more stereoisomers, for example mixtures of enantiomers and/or diastereomers, in all ratios. Thus, enantiomers are a subject of the invention in enantiomerically pure form, both as levorotatory and as dextrorotatory antipodes, in the form of racemates and in the form of mixtures of the two enantiomers in all ratios. In the case of a cis/trans isomerism the invention includes both the cis form and the trans form as well as mixtures of these forms in all ratios. The preparation of individual stereoisomers can be carried out, if desired, by separation of a mixture by customary methods, for example by chromatography or crystallization, by the use of stereochemically uniform starting materials for the synthesis or by stereoselective synthesis. Optionally a derivatization can be carried out before a separation of stereoisomers. The separation of a mixture of stereoisomers can be carried out at an intermediate step during the synthesis of a compound of Formula I or it can be done on a final racemic product. Absolute stereochemistry may be determined by X-ray crystallography of crystalline products or crystalline intermediates which are derivatized, if necessary, with a reagent containing a stereogenic center of known configuration. Alternatively, absolute stereochemistry may be determined by Vibrational Circular Dichroism (VCD) spectroscopy analysis. Where compounds of this invention are capable of tautomerization, all individual tautomers as well as mixtures thereof are included in the scope of this invention. The present invention includes all such isomers, as well as salts, solvates (which includes hydrates) and solvated salts of such racemates, enantiomers, diastereomers and tautomers and mixtures thereof.

Reference to the compounds of Formula I herein encompasses the compounds of Formulae I-XI and all embodiments and classes thereof. Reference to the compounds of this invention as those of a specific formula or embodiment, e.g., Formulae I-XI or embodiments thereof, or any other generic structural formula or specific compound described or claimed herein, is intended to encompass the specific compound or compounds falling within the scope of the Formula or embodiment, including salts thereof, particularly pharmaceutically acceptable salts, solvates (including hydrates) of such compounds and solvated salt forms thereof, where such forms are possible, unless specified otherwise.

In the compounds of Formula I, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the compounds of Formula I. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds within Formula I can be prepared without undue experimentation by conventional techniques well known to those skilled in the art or by processes analogous to those described in the Schemes and Examples herein using appropriate isotopically-enriched reagents and/or intermediates.

When the compounds of Formula I contain one or more acidic or basic groups the invention also includes the corresponding pharmaceutically acceptable salts. Thus, the compounds of Formula I which contain acidic groups can be used according to the invention as, for example but not limited to, alkali metal salts, alkaline earth metal salts or as ammonium salts. Examples of such salts include but are not limited to sodium salts, potassium salts, calcium salts, magnesium salts or salts with ammonia or organic amines such as, for example, ethylamine, ethanolamine, triethanolamine or amino acids. Compounds of Formula I which contain one or more basic groups, i.e. groups which can be protonated, can be used according to the invention in the form of their acid addition salts with inorganic or organic acids as, for example but not limited to, salts with hydrogen chloride, hydrogen bromide, phosphoric acid, sulfuric acid, nitric acid, benzenesulfonic acid, methanesulfonic acid, p-toluenesulfonic acid, naphthalenedisulfonic acids, oxalic acid, acetic acid, trifluoroacetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, etc. If the compounds of Formula I simultaneously contain acidic and basic groups in the molecule the invention also includes, in addition to the salt forms mentioned, inner salts or betaines (zwitterions). Salts can be obtained from the compounds of Formula I by customary methods which are known to the person skilled in the art, for example by combination with an organic or inorganic acid or base in a solvent or dispersant, or by anion exchange or cation exchange from other salts. The present invention also includes all salts of the compounds of Formula I which, owing to low physiological compatibility, are not directly suitable for use in pharmaceuticals but which can be used, for example, as intermediates for chemical reactions or for the preparation of pharmaceutically acceptable salts.

Furthermore, compounds of the present invention may exist in amorphous form and/or one or more crystalline forms, and as such all amorphous and crystalline forms and mixtures thereof of the compounds of Formula I are intended to be included within the scope of the present invention. In addition, some of the compounds of the instant invention may form solvates with water (i.e., a hydrate) or common organic solvents. Such solvates and hydrates, particularly the pharmaceutically acceptable solvates and hydrates, of the instant compounds are likewise encompassed within the scope of this invention, along with un-solvated and anhydrous forms.

Any pharmaceutically acceptable pro-drug modification of a compound of this invention which results in conversion in vivo to a compound within the scope of this invention is also within the scope of this invention. For example, esters can optionally be made by esterification of an available carboxylic acid group or by formation of an ester on an available hydroxy group in a compound. Similarly, labile amides can be made. Pharmaceutically acceptable esters or amides of the compounds of this invention may be prepared to act as pro-drugs which can be hydrolyzed back to an acid (or —COO— depending on the pH of the fluid or tissue where conversion takes place) or hydroxy form particularly in vivo and as such are encompassed within the scope of this invention. Examples of pharmaceutically acceptable pro-drug modifications include, but are not limited to, —C₁₋₆alkyl esters and —C₁₋₆alkyl substituted with phenyl esters.

Accordingly, the compounds within the generic structural formulas, embodiments and specific compounds described and claimed herein encompass salts, all possible stereoisomers and tautomers, physical forms (e.g., amorphous and crystalline forms), solvate and hydrate forms thereof and any combination of these forms, as well as the salts thereof, pro-drug forms thereof, and salts of pro-drug forms thereof, where such forms are possible unless specified otherwise.

The compounds of Formula I according to the invention are inhibitors of ROMK, and therefore could be used as diuretic and/or natriuretic agents. ROMK inhibitors may be used to help to increase urination and increase urine volume and also to prevent or reduce reabsorption of sodium in the kidneys leading to increased excretion of sodium and water. Therefore, the compounds could be used for treatment or prophylaxis or both of disorders that benefit from increased excretion of water and sodium from the body. Accordingly, the compounds of this invention could be used in a method for inhibiting ROMK comprising administering a compound of Formula I in a ROMK-inhibitory effective amount to a patient in need thereof. This also encompasses the use of the compounds for inhibiting ROMK in a patient comprising administering a compound of claim 1 in a therapeutically effective amount to a patient in need of diueresis, natriuresis or both. The inhibition of ROMK by the compounds of Formula I can be examined, for example, in the Thallium Flux Assay described below. Moreover, this invention also relates to the use of the compounds of Formula I or salts thereof to validate in vitro assays, for example but not limited to the Thallium Flux Assay described herein.

The compounds of this invention could be used in a method for causing diuresis, natriuresis or both, comprising administering a compound of Formula I in a therapeutically effective amount to a patient in need thereof. Therefore, the compounds of Formula I of this invention could be used in methods for treatment of, prevention of or reduction of risk for developing medical conditions that benefit from increased excretion of water and sodium, such as but not limited to one or more of hypertension, such as essential hypertension (also known as primary or idiopathic hypertension) which is a form of hypertension for which no cause can be found, heart failure (which includes both acute heart failure and chronic heart failure, the latter also known as congestive heart failure) and/or other conditions associated with excessive salt and water retention. The compounds could also be used to treat hypertension which is associated with any of several primary diseases, such as renal, pulmonary, endocrine, and vascular diseases, including treatment of patients with medical conditions such as heart failure and/or chronic kidney disease. Furthermore, the compounds of Formula I could be used in methods for treatment of, prevention of or reduction of risk for developing one or more disorders such as pulmonary hypertension, particularly pulmonary arterial hypertension (PAH), cardiovascular disease, edematous states, diabetes mellitus, diabetes insipidus, post-operative volume overload, endothelial dysfunction, diastolic dysfunction, systolic dysfunction, stable and unstable angina pectoris, thromboses, restenosis, myocardial infarction, stroke, cardiac insufficiency, pulmonary hypertonia, atherosclerosis, hepatic cirrhosis, ascitis, pre-eclampsia, cerebral edema, nephropathy, glomerulonephritis, nephrotic syndrome, acute kidney insufficiency, chronic kidney insufficiency (also referred to as chronic kidney disease, or more generally as renal impairment), acute tubular necrosis, hypercalcemia, idiopathic edema, Dent's disease, Meniere's disease, glaucoma, benign intracranial hypertension, and other conditions for which a diuretic or natriuretic or both would have therapeutic or prophylactic benefit. The compounds of the invention may be administered to a patient having, or at risk of having, one or more conditions for which a diuretic or natriuretic or both would have therapeutic or prophylactic benefit such as those described herein.

The compounds of Formula I may potentially have reduced liabilities (for example, hypo- or hyperkalemia, new onset of diabetes, dyslipidemia, etc.) over currently used clinical agents. Also the compounds may have reduced risk for diuretic tolerance, which can be a problem with long-term use of loop diuretics.

In general, compounds that are ROMK inhibitors can be identified as those compounds which, when tested, have an IC₅₀ of 5 μM or less, preferably 1 μM or less, and more preferably 0.25 μM or less, in the Thallium Flux Assay, described in more detail further below.

The dosage amount of the compound to be administered depends on the individual case and is, as is customary, to be adapted to the individual circumstances to achieve an optimum effect. Thus, it depends on the nature and the severity of the disorder to be treated, and also on the sex, age, weight and individual responsiveness of the human or animal to be treated, on the efficacy and duration of action of the compounds used, on whether the therapy is acute or chronic or prophylactic, or on whether other active compounds are administered in addition to compounds of Formula I. A consideration of these factors is well within the purview of the ordinarily skilled clinician for the purpose of determining the therapeutically effective or prophylactically effective dosage amount needed to prevent, counter, or arrest the progress of the condition. It is expected that the compound will be administered chronically on a daily basis for a length of time appropriate to treat or prevent the medical condition relevant to the patient, including a course of therapy lasting days, months, years or the life of the patient.

In general, a daily dose of approximately 0.001 to 100 mg/kg, preferably 0.001 to 30 mg/kg, in particular 0.001 to 10 mg/kg (in each case mg per kg of bodyweight) is appropriate for administration to an adult weighing approximately 75 kg in order to obtain the desired results. The daily dose is preferably administered in a single dose or can be divided into several, for example two, three or four individual doses, and may be, for example but not limited to, 0.1 mg, 0.25 mg, 0.5 mg, 0.75 mg, 1 mg, 1.25 mg, 2 mg, 2.5 mg, 5 mg, 10 mg, 20 mg, 40 mg, 50 mg, 75 mg, 100 mg, 125 mg, 150 mg, 175 mg, 200 mg, etc., on a daily basis. In some cases, depending on the potency of the compound or the individual response, it may be necessary to deviate upwards or downwards from the given daily dose. Furthermore, the compound may be formulated for immediate or modified release such as extended or controlled release.

The term “patient” includes animals, preferably mammals and especially humans, who use the instant active agents for the prophylaxis or treatment of a medical condition. Administering of the drug to the patient includes both self-administration and administration to the patient by another person. The patient may be in need of treatment for an existing disease or medical condition, or may desire prophylactic treatment to prevent or reduce the risk for developing said disease or medical condition or developing long-term complications from a disease or medical condition.

The term “therapeutically effective amount” is intended to mean that amount of a drug or pharmaceutical agent that will elicit the biological or medical response of a tissue, a system, animal or human that is being sought by a researcher, veterinarian, medical doctor or other clinician. A prophylactically effective amount is intended to mean that amount of a pharmaceutical drug that will prevent or reduce the risk of occurrence of the biological or medical event that is sought to be prevented in a tissue, a system, animal or human by a researcher, veterinarian, medical doctor or other clinician. The terms “preventing,” “prevention,” “prophylactic” and derivatives of these terms as used herein refer to administering a compound to a patient before the onset of clinical symptoms of a condition not yet present in the patient. It is understood that a specific daily dosage amount can simultaneously be both a therapeutically effective amount, e.g., for treatment of hypertension, and a prophylactically effective amount, e.g., for prevention or reduction of risk of myocardial infarction or prevention or reduction of risk for complications related to hypertension.

In the methods of treatment of this invention, the ROMK inhibitors may be administered via any suitable route of administration such as, for example, orally, parenterally, or rectally in dosage unit formulations containing conventional non-toxic pharmaceutically acceptable carriers, adjuvants and vehicles. The term parenteral as used herein includes subcutaneous injections, intravenous (IV), intramuscular, intrasternal injection or infusion techniques. Oral formulations are preferred for treatment of chronic indications such as hypertension or chronic heart failure, particularly solid oral dosage units such as pills, tablets or capsules, and more particularly tablets. IV dosing is preferred for acute treatment, for example for the treatment of acute heart failure.

This invention also provides pharmaceutical compositions comprised of a compound of Formula I and a pharmaceutically acceptable carrier which is comprised of one or more excipients or additives. An excipient or additive is an inert substance used to formulate the active drug ingredient. For oral use, the pharmaceutical compositions of this invention containing the active ingredient may be in forms such as pills, tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions. Tablets contain the active ingredient in admixture with non-toxic pharmaceutically acceptable excipients which are suitable for the manufacture of tablets. The excipients may be for example, inert diluents, such as calcium carbonate, sodium carbonate, lactose, mannitol, calcium phosphate or sodium phosphate; granulating and disintegrating agents, for example, corn starch, or alginic acid; binding agents, for example starch, gelatin or acacia, and lubricating agents, for example, magnesium stearate, stearic acid or talc.

Pharmaceutical compositions may also contain other customary additives, for example but not limited to, wetting agents, stabilizers, emulsifiers, dispersants, preservatives, sweeteners, colorants, flavorings, aromatizers, thickeners, buffer substances, solvents, solubilizers, agents for achieving a depot effect, salts for altering the osmotic pressure, coating agents or antioxidants. Oral immediate-release and time-controlled release dosage forms may be employed, as well as enterically coated oral dosage forms. Tablets may be uncoated or they may be coated by known techniques for aesthetic purposes, to mask taste or for other reasons. Coatings can also be used to delay disintegration and absorption in the gastrointestinal tract and thereby provide a sustained action over a longer period. For example, a time delay material such as glyceryl monostearate or glyceryl distearate may be employed.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredients is mixed with water or miscible solvents such as propylene glycol, PEGs and ethanol, or an oil medium, for example peanut oil, liquid paraffin, or olive oil.

Aqueous suspensions contain the active material in admixture with excipients suitable for the manufacture of aqueous suspensions. Oily suspensions may be formulated by suspending the active ingredient in a vegetable oil, for example arachis oil, olive oil, sesame oil or coconut oil, or in mineral oil such as liquid paraffin. The oily suspensions may contain a thickening agent, for example beeswax, hard paraffin or cetyl alcohol. Sweetening agents and flavoring agents may be added to provide a palatable oral preparation. These compositions may be preserved by the addition of an anti-oxidant such as ascorbic acid. Syrups and elixirs may be formulated with sweetening agents, for example glycerol, propylene glycol, sorbitol or sucrose.

The instant invention also encompasses a process for preparing a pharmaceutical composition comprising combining a compound of Formula I with a pharmaceutically acceptable carrier. Also encompassed is the pharmaceutical composition which is made by combining a compound of Formula I with a pharmaceutically acceptable carrier. Furthermore, a therapeutically effective amount of a compound of this invention can be used for the preparation of a medicament useful for inhibiting ROMK, for causing diuresis and/or natriuresis, and/or for treating, preventing or reducing the risk for any of the medical conditions described herein, in dosage amounts described herein.

The amount of active compound of Formula I and/or its pharmaceutically acceptable salts in the pharmaceutical composition may be, for example but not limited to, from about 0.1 mg to 1 g, particularly 0.1 mg to about 200 mg, more particularly from about 0.1 mg to about 100 mg, and even more particularly from about 0.1 to about 50 mg, per dose on a free acid/free base weight basis, but depending on the type of the pharmaceutical composition, potency of the active ingredient and/or the medical condition being treated, it could also be lower or higher. Pharmaceutical compositions usually comprise about 0.5 to about 90 percent by weight of the active compound on a free acid/free base weight basis.

The compounds of Formula I inhibit ROMK. Due to this property, apart from use as pharmaceutically active compounds in human medicine and veterinary medicine, they can also be employed as a scientific tool or as aid for biochemical investigations in which such an effect on ROMK is intended, and also for diagnostic purposes, for example in the in vitro diagnosis of cell samples or tissue samples. The compounds of Formula I can also be employed as intermediates for the preparation of other pharmaceutically active compounds.

One or more additional pharmacologically active agents may be administered in combination with a compound of Formula I. The additional active agent (or agents) is intended to mean a medicinal compound that is different from the compound of Formula I, and which is a pharmaceutically active agent (or agents) that is active in the body, including pro-drugs, for example esterified forms, that convert to pharmaceutically active form after administration, and also includes free-acid, free-base and pharmaceutically acceptable salts of said additional active agents when such forms are sold commercially or are otherwise chemically possible. Generally, any suitable additional active agent or agents, including but not limited to anti-hypertensive agents, additional diuretics, anti-atherosclerotic agents such as a lipid modifying compound, anti-diabetic agents and/or anti-obesity agents may be used in any combination with the compound of Formula I in a single dosage formulation (a fixed dose drug combination), or may be administered to the patient in one or more separate dosage formulations which allows for concurrent or sequential administration of the active agents (co-administration of the separate active agents). Examples of the one or more additional active agents which may be employed include but are not limited to thiazide-like diuretics, e.g., hydrochlorothiazide (HCTZ or HCT); angiotensin converting enzyme inhibitors (e.g, alacepril, benazepril, captopril, ceronapril, cilazapril, delapril, enalapril, enalaprilat, fosinopril, imidapril, lisinopril, moveltipril, perindopril, quinapril, ramipril, spirapril, temocapril, or trandolapril); dual inhibitors of angiotensin converting enzyme (ACE) and neutral endopeptidase (NEP) such as omapatrilat, sampatrilat and fasidotril; angiotensin II receptor antagonists, also known as angiotensin receptor blockers or ARBs, which may be in free-base, free-acid, salt or pro-drug form, such as azilsartan, e.g., azilsartan medoxomil potassium (EDARBI®), candesartan, e.g., candesartan cilexetil (ATACAND®), eprosartan, e.g., eprosartan mesylate (TEVETAN®), irbesartan (AVAPRO®), losartan, e.g., losartan potassium (COZAAR®), olmesartan, e.g, olmesartan medoximil (BENICAR®), telmisartan (MICARDIS®), valsartan (DIOVAN®), and any of these drugs used in combination with a thiazide-like diuretic such as hydrochlorothiazide (e.g., HYZAAR®, DIOVAN HCT, ATACAND HCT®), etc.); potassium sparing diuretics such as amiloride HCl, spironolactone, epleranone, triamterene, each with or without HCTZ; carbonic anhydrase inhibitors, such as acetazolamide; neutral endopeptidase inhibitors (e.g., thiorphan and phosphoramidon); aldosterone antagonists; aldosterone synthase inhibitors; renin inhibitors (e.g enalkrein; RO 42-5892; A 65317; CP 80794; ES 1005; ES 8891; SQ 34017; aliskiren (2(S),4(S),5 (S),7(S)-N-(2-carbamoyl-2-methylpropyl)-5-amino-4-hydroxy-2,7-diisopropyl-8-[4-methoxy-3-(3-methoxypropoxy)-phenyl]-octanamid hemifumarate) SPP600, SPP630 and SPP635); endothelin receptor antagonists; vasodilators (e.g. nitroprusside); calcium channel blockers (e.g., amlodipine, nifedipine, verapamil, diltiazem, felodipine, gallopamil, niludipine, nimodipine, nicardipine, bepridil, nisoldipine); potassium channel activators (e.g., nicorandil, pinacidil, cromakalim, minoxidil, aprilkalim, loprazolam); sympatholitics; beta-adrenergic blocking drugs (e.g., acebutolol, atenolol, betaxolol, bisoprolol, carvedilol, metoprolol, metoprolol tartate, nadolol, propranolol, sotalol, timolol); alpha adrenergic blocking drugs (e.g., doxazocin, prazocin or alpha methyldopa); central alpha adrenergic agonists; peripheral vasodilators (e.g. hydralazine); nitrates or nitric oxide donating compounds, e.g. isosorbide mononitrate; lipid lowering agents, e.g., HMG-CoA reductase inhibitors such as simvastatin and lovastatin which are marketed as ZOCOR® and MEVACOR® in lactone pro-drug form and function as inhibitors after administration, and pharmaceutically acceptable salts of dihydroxy open ring acid HMG-CoA reductase inhibitors such as atorvastatin (particularly the calcium salt sold in LIPITOR®), rosuvastatin (particularly the calcium salt sold in CRESTOR®), pravastatin (particularly the sodium salt sold in PRAVACHOL®), and fluvastatin (particularly the sodium salt sold in LESCOL®); a cholesterol absorption inhibitor such as ezetimibe (ZETIA®), and ezetimibe in combination with any other lipid lowering agents such as the HMG-CoA reductase inhibitors noted above and particularly with simvastatin (VYTORIN®) or with atorvastatin calcium); and/or with an HMG-CoA reductase inhibitor; niacin in immediate-release or controlled release forms, and particularly niacin in combination with a DP antagonist such as laropiprant and/or with an HMG-CoA reductase inhibitor; niacin receptor agonists such as acipimox and acifran, as well as niacin receptor partial agonists; metabolic altering agents including insulin sensitizing agents and related compounds for the treatment of diabetes such as biguanides (e.g., metformin), meglitinides (e.g., repaglinide, nateglinide), sulfonylureas (e.g., chlorpropamide, glimepiride, glipizide, glyburide, tolazamide, tolbutamide), thiazolidinediones also referred to as glitazones (e.g., pioglitazone, rosiglitazone), alpha glucosidase inhibitors (e.g., acarbose, miglitol), dipeptidyl peptidase inhibitors, (e.g., sitagliptin (JANUVIA®), alogliptin, vildagliptin, saxagliptin, linagliptin, dutogliptin, gemigliptin), ergot alkaloids (e.g., bromocriptine), combination medications such as JANUMET® (sitagliptin with metformin), and injectable diabetes medications such as exenatide and pramlintide acetate; phosphodiesterase-5 (PDE5) inhibitors such as sildenafil (REVATIO®, VIAGRA®), tadalafil (CIALIS®, ADCIRCA®) vardenafil HCl (LEVITRA®); or with other drugs beneficial for the prevention or the treatment of the above-mentioned diseases including but not limited to diazoxide; and including the free-acid, free-base, and pharmaceutically acceptable salt forms, pro-drug forms (including but not limited to esters), and salts of pro-drugs of the above medicinal agents where chemically possible. Trademark names of pharmaceutical drugs noted above are provided for exemplification of the marketed form of the active agent(s); such pharmaceutical drugs could be used in a separate dosage form for concurrent or sequential administration with a compound of Formula I, or the active agent(s) therein could be used in a fixed dose drug combination including a compound of Formula I.

Several methods for preparing the compounds of this invention are described in the following Schemes and Examples. Starting materials and intermediates are purchased or are made using known procedures, or as otherwise illustrated. Some frequently applied routes to the compounds of Formula I are described in Schemes 1-4 that follow. In some cases the order of carrying out the reaction steps in the schemes may be varied to facilitate the reaction or to avoid unwanted reaction products.

The figure below represents the N-containing multicyclic heteroaromatic group as it is used in the following schemes to illustrate chemical reactions that are used to synthesize the group Z from synthetic intermediates that contain the N-containing multicyclic heteroaromatic groups:

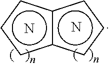

Schemes 1-4 illustrate the chemical reactions that convert synthetic intermediates that contain N-containing multicyclic heteroaromatic groups to the groups Z. “N-containing multicyclic heteroaromatic” groups are defined earlier in this application in the Detailed Description. The figure shown above is not meant to represent any specific chemical structure or to limit N-containing multicyclic heteroaromatic groups to bicyclic rings (some groups Z are tricyclic).

Several methods for preparing the compounds of this invention are described in the examples. Starting materials and intermediates are purchased, made using known procedures, or as otherwise illustrated. Some frequently applied routes to the compounds of Formula I are also described by the Schemes as follows. In some cases the order of carrying out the steps of reaction schemes may be varied to facilitate the reaction or to avoid unwanted reaction products.

Starting from a spirobicyclic core, important steps for the synthesis of the inventive compounds include the opening of an epoxide and the formation of a C—N bond. As outlined in Scheme 1, the mono protected diaza spirobicyclic core 2 reacts with epoxide 1 to give compound 3; removal of the Boc protecting group leads to compound 4, and subsequent C—N coupling or SnAr substitution provides the final compound 5.

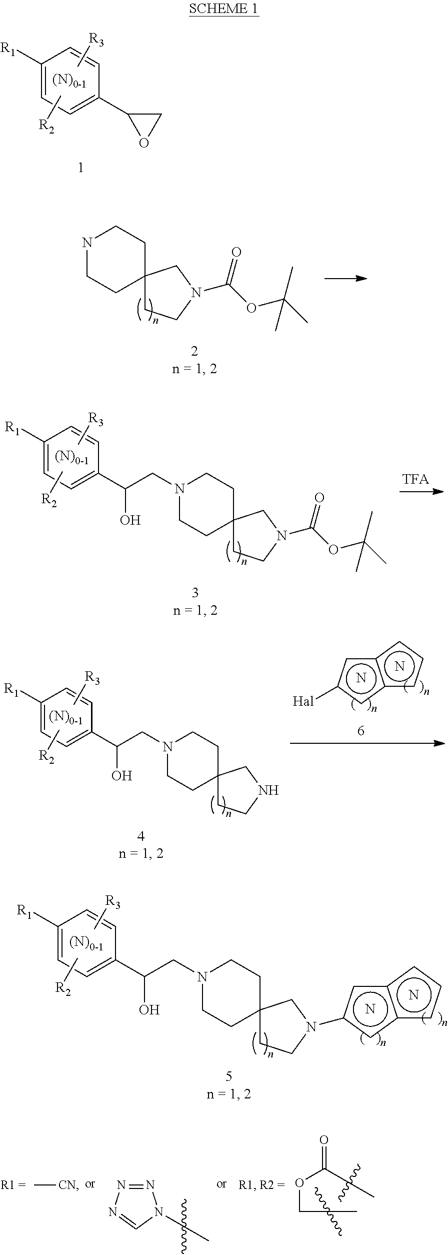

Similar chemistry can be applied to a spirobicyclic lactam core 7 (Scheme 2) to afford final compound 9 (Scheme 2).

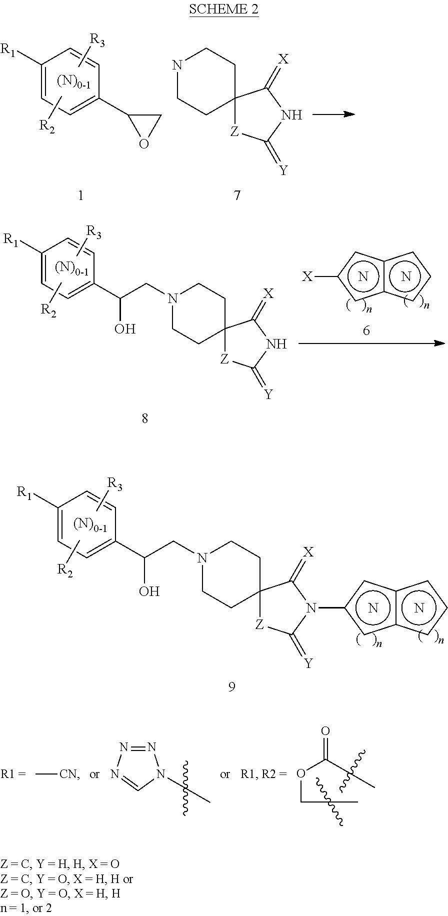

Alternatively, as outlined in Schemes 3 and 4, the compounds can be synthesized by a two stage process where the mono-protected spirobicyclic core, such as 10 (Scheme 3) or 14 (Scheme 4) is first reacted with halide 6 followed by deprotection of Boc group, and then the epoxide ring is opened as illustrated in Scheme 3 and Scheme 4.

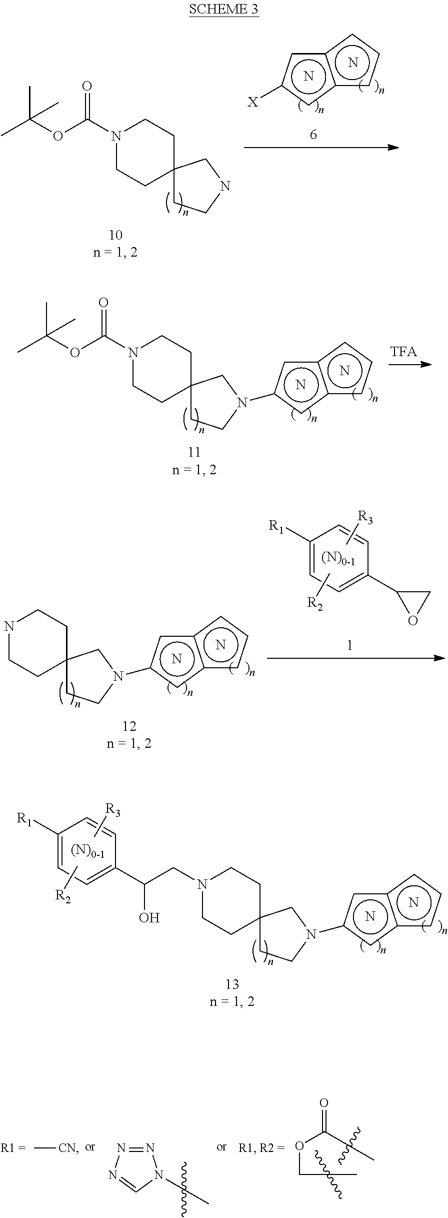

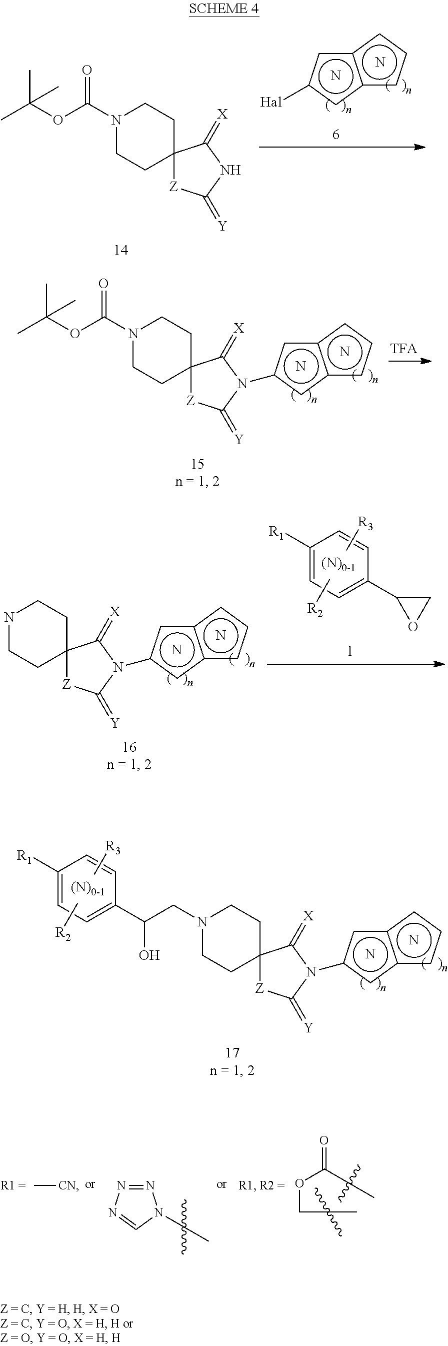

### General Procedures:

Reactions sensitive to moisture or air were performed under nitrogen or argon using anhydrous solvents and reagents. The progress of reactions was determined by either analytical thin layer chromatography (TLC) usually performed with E. Merck pre-coated TLC plates, silica gel 60F-254, layer thickness 0.25 mm or liquid chromatography-mass spectrometry (LC-MS). Typically the analytical LC-MS system used consisted of a Waters ZQ™ platform with electrospray ionization in positive ion detection mode with an Agilent 1100 series HPLC with autosampler. The column was usually a Water Xterra MS C18, 3.0×50 mm, 5 μm. The flow rate was 1 mL/min, and the injection volume was 10 μL. UV detection was in the range 210-400 nm. The mobile phase consisted of solvent A (water plus 0.06% TFA) and solvent B (acetonitrile plus 0.05% TFA) with a gradient of 100% solvent A for 0.7 min changing to 100% solvent B over 3.75 min, maintained for 1.1 min, then reverting to 100% solvent A over 0.2 min. Preparative HPLC purifications were usually performed using a mass spectrometry directed system. Usually they were performed on a Waters Chromatography Workstation configured with LC-MS System Consisting of: Waters ZQ™ single quad MS system with Electrospray Ionization, Waters 2525 Gradient Pump, Waters 2767 Injecto/Collector, Waters 996 PDA Detector, the MS Conditions of: 150-750 amu, Positive Electrospray, Collection Triggered by MS, and a Waters SUNFIRE® C-18 5 micron, 30 mm (id)×100 mm column. The mobile phases consisted of mixtures of acetonitrile (10-100%) in water containing 0.1% TFA. Flow rates were maintained at 50 mL/min, the injection volume was 1800 μL, and the UV detection range was 210-400 nm. Mobile phase gradients were optimized for the individual compounds. Reactions performed using microwave irradiation were normally carried out using an Emrys Optimizer manufactured by Personal Chemistry, or an Initiator manufactured by Biotage. Concentration of solutions was carried out on a rotary evaporator under reduced pressure. Flash chromatography was usually performed using a Biotage® Flash Chromatography apparatus (Dyax Corp.) on silica gel (32-63 mM, 60 Å pore size) in pre-packed cartridges of the size noted. ¹H NMR spectra were acquired at 500 MHz spectrometers in CDCl₃ solutions unless otherwise noted. Chemical shifts were reported in parts per million (ppm). Tetramethylsilane (TMS) was used as internal reference in CD₃Cl solutions, and residual CH₃OH peak or TMS was used as internal reference in CD₃OD solutions. Coupling constants (J) were reported in hertz (Hz). Chiral analytical chromatography was performed on one of CHIRALPAK® AS, CHIRALPAK®AD, CHIRALCEL®OD, CHIRALCEL® IA, or CHIRALCEL® OJ columns (250×4.6 mm) (Daicel Chemical Industries, Ltd.) with noted percentage of either ethanol in hexane (% Et/Hex) or isopropanol in heptane (% IPA/Hep) as isocratic solvent systems. Chiral preparative chromatography was conducted on one of CHIRALPAK AS, of CHIRALPAK AD, CHIRALCEL®OD, CHIRALCEL®IA, CHIRALCEL® OJ columns (20×250 mm) (Daicel Chemical Industries, Ltd.) with desired isocratic solvent systems identified on chiral analytical chromatography or by supercritical fluid (SFC) conditions.

Abbreviations and acronyms that may be used herein include: —C(O)CH₃ (Ac); —OC(O)CH₃ (OAc); acetic acid (AcOH; HOAc); 1-chloroethylchloroformate (ACE-Cl); 2,2′-bis(diphenylphosphino)-1,1′-binaphthyl (BINAP); benzyl (Bn); t-butyloxycarbonyl (Boc or BOC); di-t-butyl dicarbonate ((BOC)₂O, Boc₂O); benzyloxycarbonyl (Cbz); n-butyl (Bu); tert-butyl (t-butyl); cyclopentyl methyl ether (CPME); carbonyldiimidazole (CDI); diethylaminosulfur trifluoride (DAST); dibenzylideneacetone (dba); 1,8-diazabicyclo[5.4.0]undec-7-ene (DBU); 1,2-dichloroethane (DCE); dichloromethane (DCM); diethyl amine (DEA); dimethoxyethane (DME); diisobutylaluminium hydride (DIBAL-H); N,N-diisopropylethylamine (DIEA, DIPEA, Hunig's base); dioxane is 1,4-dioxane; di-isopropylamine (DIPA); 1,1′-bis(diphenylphosphino)ferrocene (dppf, DPPF); Dess-Martin Periodinane (DMP; 1,1,1-triacetoxy-1,1-dihydro-1,2-benziodoxol-3 (1H)-one); dimethylsulfide (DMS); dimethylsulfoxide (DMSO); N;N-dimethylformamide (DMF); 4-dimethylaminopyridine (DMAP); dimethylacetamide (DMA; DMAC); 1,3-bis(diphenylphosphino)propane (DPPP); (Oxydi-2,1-phenylene)bis(diphenylphosphine) (DPEPhos); diphenyl phosphoryl azide (DPPA); ethyl (ET); ethyl acetate (EtOAc or EA); ethanol (EtOH); diethyl ether (ether or Et₂O); 1-ethyl-3-(3-dimethylaminopropyl)carbodiimide (EDC, EDAC or EDCI); 2-(7-Aza-1H-benzotriazole-1-yl)-1,1,3,3-tetramethyluronium hexafluorophosphate (HATU); hexane (Hex); hexamethylphosphoramide (HMPA); 1-hydroxybenzotriazole hydrate (HOBt); isopropanol (IPA or iPrOH); isopropyl acetate (IPAc); potassium bis(trimethylsilyl)amide (KHMDS); lithium aluminum hydride (LAH); lithium diisopropylamide (LDA); 3-chloroperoxybenzoic acid (mCPBA); methanol (MeOH); CH₃SO₂— (mesyl or Ms); methane sulfonyl chloride or mesyl chloride (MsCl); methanesulfonic acid (MsOH); methyl (Me); methyl tert-butyl ether (MTBE); nicotinamide adenine dinucleotide phosphate (NADP); N-bromo succinimide (NBS); N-chlorosuccinimide (NCS); N-iodosuccinimide (NIS); N-methylmorpholine-N-oxide (NMO); N-methyl morpholine (NMP); sodium hexamethyldisilazide (NaHMDS); sodium triacetoxyborohydride (NaBH(OAc)₃); pyridinium chlorochromate (PCC); phenyl (Ph); petroleum ether (PE or petrol ether); tetrakis(triphenylphosphine)palladium (Pd(PPh₃)₄); tris(dibenzylidineacetone)dipalladium (Pd₂(dba)₃); Pd(dppf)Cl₂ or PdCl₂(dppf) is 1,1′-Bis(diphenylphosphino)ferrocene]-dichloropalladium(II) which may be complexed with CH₂Cl₂; Chloro-(2-Dicyclohexylphosphino-2′,6′-diisopropoxy-1,1′-biphenyl) [2-(2-aminoethyl)phenyl]palladium(II)-methyl-t-butyl ether adduct (RuPhos precatalyst); tetra-n-butylammonium fluoride (TBAF); tetrabutylammonium tribromide (TBATB); tert-butyldimethylsilyl chloride (TBS-Cl); triethylamine (TEA); trifluoroacetic acid (TFA); —SO₂CF₃ (Tf); trifluoromethanesulfonic acid (triflic acid, TfOH); trifluoromethanesulfonic anhydride (triflic anhydride, (Tf)₂O); 2-tetrahydrofuran (THF); N,N,N′,N′-tetramethylethylenediamine (TMEDA); p-toluenesulfonic acid (TsOH or PTSA); dicyclohexylphosphino-2′,4′,6′-triisopropylbiphenyl (X-Phos); diethylaminodifluorosulfinium tetrafluoroborate (XtalFluor-E®); 4,5-bis(diphenylphosphino)-9,9-dimethylxanthene (Xantphos). Additional abbreviations and acronyms are: racemic or racemate (rac.); starting material (SM); round-bottom flask (RB or RBF); aqueous (aq); saturated aqueous (sat'd); saturated aqueous sodium chloride solution (brine); maximum temperature (Tₘₐₓ); medium pressure liquid chromatography (MPLC); high pressure liquid chromatography (HPLC); preparative HPLC (prep-HPLC); reverse phase high pressure liquid chromatorgraphy (RP-HPLC); ionization energy (IE); flash chromatography (FC); liquid chromatography (LC); solid phase extraction (SPE); supercritical fluid chromatography (SFC); 2-dicyclohexylphosphino-2′,6′-dimethoxybiphenyl (SPhos); thin layer chromatography (TLC); preparative TLC (prep-TLC); mass spectrum (ms or MS); liquid chromatography-mass spectrometry (LC-MS, LCMS or LC/MS); column volume (CV); room temperature (rt, r.t. or RT); hour(s) (h or hr); minute(s) (min); retention time (Rₜ); gram(s) (g); milligram(s) (mg); milliliter(s) (mL); microliter(s) (L); millimole (mmol); volume:volume (V/V). CELITE® is a trademark name for diatomaceous earth, and SOLKA FLOC® is a trademark name for powdered cellulose. X or x may be used to express the number of times an action was repeated (e.g., washed with 2×200 mL 1N HCl), or to convey a dimension (e.g., the dimension of a column is 30×250 mm).

The following are representative procedures for the preparation of the compounds used in the following Examples, or which can be substituted for the compounds used in the following Examples, which may not be commercially available.

### Intermediate 1

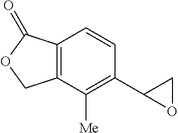 [SMILES: Cc1c(C2CO2)ccc2c1COC2=O]

### 4-methyl-5-oxiran-2-yl-2-benzofuran-1 (3H)-one

### Step A: 5-ethenyl-4-methyl-2-benzofuran-1(3H)-one

5-Bromo-4-methyl-2-benzofuran-1(3H)-one (598 mg, 4.47 mmol), potassium vinyl trifluoroborate (507 mg, 2.23 mmmol), PdCl₂(dppf)-CH₂Cl₂Adduct (182 mg, 0.223 mmmol), and TEA (0.622 mL, 4.47 mmol) were added to 10 mL ethanol in a 20 mL microwave tube. The tube was sealed and degassed, then heated to 140° C. for 20 min. Analysis by LC-MS showed product peak. The reaction mixture was diluted with ethyl acetate, washed with brine twice, dried and evaporated to dryness. The crude product was purified by MPLC chromatography using a 120 g RediSep® column and 0-80% EtOAc/hexane solvent system to yield 5-ethenyl-4-methyl-2-benzofuran-1(3H)-one. ¹H-NMR (500 MHz, CDCl₃): δ ppm 7.76 (d, J=8 Hz, 1H), 7.03 (dd, J=11, 17 Hz, 1H), 5.84 (d, J=17 Hz, 1H), 5.55 (d, J=11 Hz, 1H), 5.29 (s, 2H), 2.34 (s, 3H); LC-MS: M+1=175; Step B: 4-methyl-5-oxiran-2-yl-2-benzofuran-1(3H)-one: 5-ethenyl-4-methyl-2-benzofuran-1(3H)-one (1.46 g, 8.38 mmol) was added to DCM (25 mL) at 0° C. then mCPBA (2.89 g, 16.8 mmol) was added and the mixture was stirred at RT overnight. The reaction mixture was washed once each with saturated aqueous Na₂S₂O₃, NaHCO₃, and brine. The organic layer was dried over Na₂SO₄, filtered, and evaporated to dryness. The crude material was purified by MPLC chromatography through 120 g RediSep® column eluting with 0-80% EtOAc/hexane solvent system to yield target 4-methyl-5-oxiran-2-yl-2-benzofuran-1(3H)-one. ¹H-NMR (500 MHz, CDCl₃): δ ppm 7.77 (d, J=8 Hz, 1H), 7.43 (d, J=8 Hz, 1H), 5.30 (s, 2H), 4.12 (s, 1H), 3.27 (t, J=4 Hz, 1H), 2.735 (dd, J=2.2, 5.5 Hz, 1H), 2.43 (s, 3H). LC-MS: M+1=191.

### Intermediates 1A and 1B (Method 1)

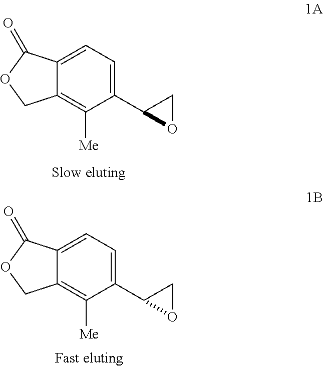 [SMILES: Cc1c([C@H]2CO2)ccc2c1COC2=O ; Cc1c([C@@H]2CO2)ccc2c1COC2=O]

### 1A: 4-methyl-5-[(2S)-oxiran-2-yl]-2-benzofuran-1 (3H)-one

### 1B: 4-methyl-5-[(2R)-oxiran-2-yl]-2-benzofuran-1 (3H)-one

Racemic 4-methyl-5-oxiran-2-yl-2-benzofuran-1(3H)-one was resolved on a CHIRALPAK® AD-H column (5×25 cm) under supercritical fluid chromatography (SFC) conditions on a Berger MGIII preparative SFC instrument. The racemate was diluted to 50 mg/mL in 1:1 DCM:MeOH. The separation was accomplished using 10% EtOH/CO₂, flow rate 200 mL/min, 100 bar, 25° C. 500 μl injections were spaced every 2.12 mins. The fast epoxide (4-methyl-5-[(2R)-oxiran-2-yl]-2-benzofuran-1(3H)-one, 1B) eluted first, and the slow epoxide (4-methyl-5-[(2S)-oxiran-2-yl]-2-benzofuran-1(3H)-one, 1A) eluted second.

Alternatively, the resolution could also be achieved using a mobile phase of 8% MeOH/98% CO₂ with a flow rate of 100 mL/min. In that case the sample was prepared by dissolving in methanol, 20 mg/mL, and using a 1 mL volume per injection. After separation, the fractions were dried off via rotary evaporator at bath temperature 40° C.

The absolute stereochemistry of each enantiomer was inferred based on the X-ray crystal structure determination of a final compound made with 1B and by Mosher ester and Trost ester HNMR analysis of esters made starting from 1B. Both epoxide isomers find utility in the present invention.

### Intermediate 1B (Method 2)

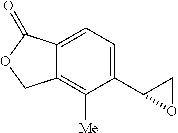 [SMILES: Cc1c([C@@H]2CO2)ccc2c1COC2=O]

### 4-methyl-5-[(2R)-oxiran-2-yl]-2-benzofuran-1(3H)-one

### Step A: 3-hydroxymethyl-2-methyl phenol

To a 5 L 3-neck round bottom flask equipped with overhead stirrer was charged NaBH₄ (87.0 g, 2.30 mol) and THF (3.0 L) and the resulting slurry was cooled to 10° C. To the slurry 3-hydroxy-2-methyl benzoic acid (175 g, 1.15 mol) was added portionwise over 20 min (Tₘₐₓ 17° C.). A stirrable slurry formed, which was aged for an additional 45 min at 10-15° C. after which BF₃—OEt₂ (321 mL, 2.53 mol) was added slowly over 1.5 hours. The slurry was aged at 10° C.-15° C. for 2 h and then assayed for reaction completion (98.5% conversion). The slurry was cooled to <10° C. and quenched with 931 mL MeOH slowly over 1.5 h (gas evolution). The resulting slurry was aged overnight at RT. The batch was cooled to <10° C. then quenched with 1 N HCl (1.5 L) to get a homogeneous solution (pH solution ˜1), which was aged for 30 min and then the organic solvents were removed by rotary evaporation to approximately 1.8 L of total reaction volume (bath temperature was set to 50° C.; internal temp of concentrate after rotary evaporation was approximately 40° C.). The slurry was held at 45° C. for 30 min then cooled slowly to 15° C. The solids were filtered and washed with cold (15° C.) water (2×300 mL), providing 3-hydroxymethyl-2-methyl phenol. ¹H-NMR (400 MHz, DMSO-d₆): δ 9.11 (s, 1H), 6.95 (t, J=7.8 Hz, 1H), 6.82 (d, J=7.4 Hz, 1H), 6.71 (d, J=7.8 Hz, 1H), 4.93 (t, J=5.5 Hz, 1H), 4.44 (d, J=5.5 Hz, 2H), 2.06 (s, 3H).

### Step B: 4-bromo-3-hydroxymethyl-2-methyl phenol

3-Hydroxymethyl-2-methyl phenol (113.9 g, 824.0 mmol) was dissolved in a mixture of acetonitrile (850 mL) and trifluoroacetic acid (750.0 mL, 9,735 mmol) in a 3-neck 5-L flask under nitrogen. The reaction mixture was cooled to −33° C. N-bromosuccinimide (141 g, 791 mmol) was added over 15 minutes, with the temperature during addition in the range of about −35 to about −33° C. The reaction mixture was allowed to stir for an additional 15 min during which time the temperature decreased to −40° C.

The cooling bath was removed, and potassium carbonate (741.0 g, 5,358 mmol) diluted with water to a total of 1.0 L was added. The evolution of gas was observed and the temperature increased to 25° C. MTBE (1.5 L) was added and the reaction mixture was transferred to a separatory funnel. The layers were separated. The aqueous layer was diluted with water (500 mL) and extracted with MTBE (1 L)+EtOAc (500 mL), and then MTBE (500 mL)+EtOAc (250 mL). The combined organic layers were washed with water (240 mL) and dried over sodium sulfate. The sodium sulfate was removed by filtration, washed with additional MTBE and concentrated under reduced pressure. MTBE (684 mL, 2 volumes) was added and the resulting suspension was heated to 40° C. to produce a homogeneous solution. The solution was allowed to cool to room temperature. Six volumes of heptane were added and the resulting suspension was stirred overnight. The suspension was filtered, and the crystals were washed with 4:1 heptane: MTBE (500 mL), followed by heptane (500 mL). The solid was dried under vacuum, providing 4-bromo-3-hydroxymethyl-2-methyl phenol. ¹H NMR (400 MHz, DMSO-d₆): δ 9.52 (s, 1H), 7.21 (d, J=8.6 Hz, 1H), 6.71 (d, J=8.6 Hz, 1H), 4.88 (t, J=5.1 Hz, 1H), 4.59 (d, J=5.1 Hz, 2H), 2.23 (s, 3H)

### Step C: 5-hydroxy-4-methyl-3H-isobenzofuran-1-one

4-Bromo-3-hydroxymethyl-2-methyl phenol (100 g, 461 mmol), CuCN (83.0 g, 921 mmol), and DMF (500 mL) were charged to a 2 L 3-neck flask equipped with overhead stirrer, N₂ inlet, and condenser. The solution was sparged with N₂ for 15 min then heated to 145° C. to obtain a homogeneous solution. The solution was aged at 145° C. for 2 h and then the reaction mixture was cooled to 95° C. 41.5 mL of water was added (sparged with N₂) and the reaction aged for 20 h. The reaction was cooled to RT then the solids filtered through SOLKA FLOC® and the cake washed with 50 mL DMF. The filtrate from the DMF was added to a 3 L flask containing 1 L EtOAc. A precipitate coating formed in bottom of flask. The DMF/EtOAc suspension was filtered through SOLKA FLOC® and the cake was washed with 250 mL EtOAc. The resulting filtrate was washed with 5% brine solution (3×500 mL). The aqueous layers were extracted with 500 mL EtOAc and the combined organics were dried over MgSO₄, filtered and evaporated. The solids were slurried in 250 mL MTBE at RT then filtered and washed with 100 mL MTBE. The solids were dried under vacuum at RT, providing 5-hydroxy-4-methyl-3H-isobenzofuran-1-one. ¹H NMR (400 MHz, DMSO-d₆): δ 10.52 (s, 1H), 7.51 (d, J=8.3 Hz, 1H), 6.99 (d, J=8.3 Hz, 1H), 5.28 (s, 2H), 2.07 (s, 3H).

### Step D: 4-methyl-1-oxo-1,3-dihydroisobenzofuran-5-yl trifluoromethanesulfonate

5-Hydroxy-4-methyl-3H-isobenzofuran-1-one (46.8 g, 285 mmol) was suspended in dichloromethane (935 mL) in 2-L roundbottom flask equipped with overhead stirrer under nitrogen. Triethylamine (59.5 mL, 427 mmol) was added and the reaction mixture was cooled in an ice bath to 3.8° C. Trifluoromethanesulfonic anhydride (67.4 mL, 399 mmol) was added via addition funnel over 50 min, keeping the temperature <10° C. After stirring the reaction mixture for an additional 15 min, the reaction mixture was quenched with water (200 mL) and then stirred with DARCO® KB (activated carbon, 25 g) for 15 min. The biphasic mixture was filtered over SOLKA FLOC®, washing with additional dichloromethane, and transferred to a separatory funnel, whereupon it was diluted with additional water (300 mL). The layers were separated, and the organic layer was washed with water (500 mL) and 10% brine (200 mL). The dichloromethane solution was dried over sodium sulfate, filtered and evaporated. The orange-red solid was adsorbed onto silica gel (27.5 g) and eluted through a pad of silica gel (271 g) with 25% ethyl acetate/hexanes. The resulting solution was concentrated under vacuum with the product crystallizing during concentration. The suspension was filtered, the solid washed with heptane and dried under vacuum and nitrogen, providing trifluoromethanesulfonic acid 4-methyl-1-oxo-1,3-dihydro-isobenzofuran-5-yl ester. ¹H NMR (400 MHz, CDCl₃): δ 7.87 (d, J=8.4 Hz, 1H), 7.47 (d, J=8.4 Hz, 1H), 5.32 (s, 2H), 2.41 (s, 3H)

### Step E: 5-(1-butoxy-vinyl)-4-methyl-3H-isobenzofuran-1-one

Trifluoromethanesulfonic acid 4-methyl-1-oxo-1,3-dihydro-isobenzofuran-5-yl ester (63.0 g, 213 mmol), DMF (315 mL), butyl vinyl ether (138 mL, 1063 mmol)) were charged to a 1 L 3-neck flask and then Et₃N (35.6 mL, 255 mmol) were added. The solution was sparged with N₂ for 20 min. To the solution was added Pd(OAc)₂ (1.19 g., 5.32 mmol) and DPPP (2.41 g., 5.85 mmol) and sparged for an additional 10 min then heated to 80° C. After aging for 1 hr, the solution was cooled to <10° C., quenched with 630 mL EtOAc, washed with 5% NH₄Cl (2×315 mL), 10% brine (2×315 mL), dried over MgSO₄, filtered, and concentrated by rotary evaporation and flushed with EtOAc (3×100 mL) to remove excess butyl vinyl ether, and provided crude 5-(1-butoxy-vinyl)-4-methyl-3H-isobenzofuran-1-one. ¹H NMR (400 MHz, DMSO-d₆): δ 7.67 (d, J=7.7 Hz, 1H), 7.48 (d, J=7.7 Hz, 1H), 5.42 (s, 2H), 4.54 (d, J=2.3 Hz, 1H), 4.27 (d, J=2.3 Hz, 1H), 3.85 (t, J=6.4 Hz, 2H), 2.27 (s, 3H), 1.71-1.64 (m, 2H), 1.46-1.37 (m, 2H), 0.92 (t, J=7.4 Hz, 3H)

### Step F: 5-(2-bromo-acetyl)-4-methyl-3H-isobenzofuran-1-one

Crude 5-(1-butoxy-vinyl)-4-methyl-3H-isobenzofuran-1-one (55.8 g) and THF (315 mL) were added to a 1 L 3-neck flask equipped with overhead stirrer. The solution was cooled to <5° C. after which water (79 mL) was added and the solution was maintained at <5° C. NBS (41.6 g) was then added portion-wise while maintaining Tₘₐₓ of 19° C. The solution was then warmed to RT for 30 minutes. HBr (48%, 0.241 mL) was added and the reaction was aged at RT for approximately 1 h after which 236 mL water was then added to the batch. A water bath is used to maintain temp at 20° C. Another 315 mL of water was added (solvent composition 1:2 THF:water) and the slurry was cooled to 15° C. The resulting solids were filtered and washed with cold 1:2 THF:water (15° C.): 150 mL displacement wash followed by 100 mL slurry wash. The solids were dried under vacuum at RT to provide 5-(2-bromo-acetyl)-4-methyl-3H-isobenzofuran-1-one. ¹H NMR (400 MHz, DMSO-d₆): δ 7.99 (d, J=7.8 Hz, 1H), 7.82 (d, J=7.8 Hz, 1H), 5.49 (s, 2H), 4.92 (s, 2H), 2.33 (s, 3H)

### Step G: 4-methyl-5-[(2R)-oxiran-2-yl]-2-benzofuran-1 (3H)-one

5-(2-Bromo-acetyl)-4-methyl-3H-isobenzofuran-1-one (48.8 g., 181 mmol) was charged to a 5 L 3 neck round bottom equipped with overhead stirrer, thermocouple, and heating mantle. 2-Propanol (1.22 L) was added, followed by 610 mL of pH 7 0.1M potassium phosphate buffer. Buffer solution (610 mL) was charged to a 1.0 L erlenmeyer, and 2.44 g of NADP was added to the Erlenmeyer and swirled to dissolve. A reducing enzyme, KRED MIF-20 (2.44 g) (available from Codexis, Inc., 200 Penobscot Drive, Redwood City, Calif. 94063, www.codexis.com, tel. 1-650-421-8100) was added to the Erlenmeyer flask and the mixture was swirled to dissolve the solids. The resulting solution was added to the 5 L round bottom, which was then heated to 28° C. and aged for 6 hours, at which point the reaction was cooled to RT and triethylamine (50.2 mL, 360 mmol) was added. The resulting solution was aged at 40° C. for 1 h. The light slurry solution was cooled to RT, after which 122 g NaCl was added. The solution was aged at RT then extracted with 1.22 L IPAc. The aqueous layer was re-extracted with 400 mL IPAc and the combined organics were washed with 400 mL 20% brine solution, dried over MgSO₄, filtered and concentrated by rotary evaporation. The resulting solids were taken up in 100 mL IPAc (thick slurry). Hexanes were added (400 mL) and the suspension aged at RT then filtered and washed w/5:1 hexanes:IPAc solution (150 mL). The crystalline solids were dried under vacuum at RT to provide 4-methyl-5-[(2R)-oxiran-2-yl]-2-benzofuran-1(3H)-one. ¹H NMR (400 MHz, CDCl₃): δ 7.75 (d, J=8.1 Hz, 1H), 7.42 (d, J=8.1 Hz, 1H), 5.28 (s, 2H), 4.10 (dd, J=4.0, 2.8, 1H), 3.26 (dd, J=5.6, 4.0, 1H), 2.72 (dd, J=5.6, 2.8, 1H), 2.42 (s, 3H).

### Intermediate 2A and 2B

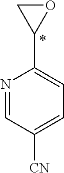 [SMILES: N#Cc1ccc(C2CO2)nc1]

### Step A: 6-Vinylnicotinonitrile

To a stirring solution of 6-bromonicotinonitrile (2.0 g, 10.9 mmol) in EtOH (70 ml) were added bis[(diphenylphosphino)ferrocene]dichloropalladium (II), complex with dichloromethane (0.892 mg, 0.10 mmol), potassium vinyl trifluoroborate (2.93 g, 21.9 mmol), triethylamine (3.0 ml, 21.9 mmol), and water (0.5 mL). The reaction mixture was heated to reflux. Upon completion as determined by reverse phase HPLC-MS (1-2 h) and TLC (elute: 10% ethyl acetate in hexanes), the reaction was cooled to room temperature, and then diluted with water and extracted with EtOAc. The combined organic layers were washed with brine and dried over MgSO₄. The extracts were concentrated and chromatographed over a column of SiO₂ (0-20% EtOAc/hexanes as eluent). Evaporation of the solvent yielded 6-vinylnicotinonitrile. LC/MS: [(M+1)]⁺=131; ¹H NMR (500 MHz, CDCl₃) δ 8.85 (s, 1H), 7.94-7.93 (m, 1H), 6.89-6.83 (m, 1H), 7.45 (d, J=8.2 Hz, 1H), 6.85 (dd, J=10.8 Hz, 1H), 6.42 (d, J=17.4 Hz, 1H).

### Step B: 6-(oxiran-2-yl)nicotinonitrile

A solution of 6-vinylnicotinonitrile (0.742 g, 5.70 mmol) in a 2:1 ratio of water:t-BuOH (30 mL) was treated with N-bromosuccinimide in portions over 5 minutes (1.07 g, 5.99 mmol) and stirred at 40° C. for 1 h. After cooling to 5° C., the reaction was basified with dropwise addition of solution of sodium hydroxide (0.684 g in 5 ml of water, 17.1 mmol) and stirred for another 1 h. The reaction mixture was poured into water (10 ml) and extracted with EtOAc (2×50 mL). The combined organic layers were washed with saturated aqueous NaCl (1×30 ml) and dried over MgSO₄. Evaporation of the solvent and purification over SiO2 (0-30% EtOAc/hexanes as eluent) provided 6-(oxiran-2-yl)nicotinonitrile.

LC/MS: [(M+1)]⁺=147; ¹H NMR (500 MHz, CDCl₃), δ 8.87 (s, 1H), 7.99 (d, J=8.1 Hz, 1H), 7.40 (d, J=8.1 Hz, 1H), 4.11 (s, 1H), 4.08 (dd, J=2.6 Hz, J=2.3 Hz, 1H), 3.29 (m, 1H), 2.94 (m, 1H). Resolution of the epoxide was carried out (prep SFC, 160 mL/min., 10% MeOH in SC CO₂, AD-H) to provide:

Fast eluted isomer A: (M+1)⁺=147
Slow eluted isomer B: (M+1)⁺=147

### Intermediate 3

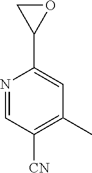

4-Methyl-6-(oxiran-2-yl)nicotinonitrile was prepared in a similar fashion to that described for the synthesis if Intermediate 2 starting from 6-chloro-4-methylnicotinonitrile. LC/MS: [(M+1)]⁺=161.

### Intermediate 4A and 4B

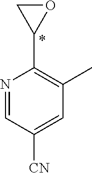

5-Methyl-6-(oxiran-2-yl)nicotinonitrile was prepared in a similar fashion to that described for the synthesis of Intermediate 2 starting from 6-chloro-5-methylnicotinonitrile. LC/MS: [(M+1)]⁺=161. Resolution of the epoxide was carried out on prep SFC in a similar fashion to that described for INTERMEDIATE 2A and 2B to provide fast eluted 4A and slow eluted 4B.

### Intermediate 5

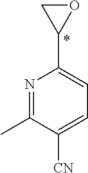

2-Methyl-6-(oxiran-2-yl)nicotinonitrile was prepared in a similar fashion to that described for the synthesis of INTERMEDIATE 2 starting from 6-chloro-2-methylnicotinonitrile. LC/MS: [(M+1)]⁺=161. Resolution of the epoxide was carried out on prep SFC in a similar fashion to that described for INTERMEDIATE 2A and 2B to provide fast eluted 5A and slow eluted 5B.

### Intermediates 6A and 6B

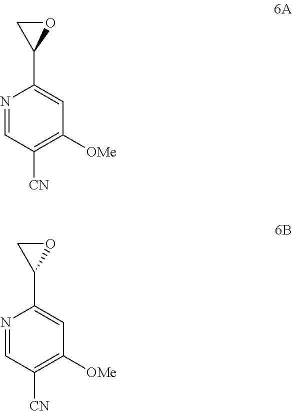 [SMILES: COc1cc([C@H]2CO2)ncc1C#N ; COc1cc([C@@H]2CO2)ncc1C#N]

### (S)-4-methoxy-6-(oxiran-2-yl)nicotinonitrile (6A) and (R)-4-methoxy-6-(oxiran-2-yl)nicotinonitrile (6B)

### Step A: 5-bromo-2-chloro-4-methoxypyridine

To a solution of 2-chloro-4-methoxypyridine (10.0 g, 69.7 mmol) in 50 mL of sulfuric acid at 0° C. was added NBS. The reaction mixture was allowed to stir and warm up to room temperature for 2 h and then heated at 60° C. for 5 h. Next, the reaction mixture was cooled to room temperature, neutralized with 1 N NaOH (pH˜7), diluted with water (50 ml) and the aqueous layer was extracted with ethyl acetate (2×100 mL). The organic layers were washed with water (2×50 mL), saturated NaHCO₃, brine, dried over Mg₂SO₄ and concentrated to provide an oil, which was chromatographed to give 5-bromo-2-chloro-4-methoxypyridine eluting with 0-25% EtOAc/hexanes. ¹HNMR (500 MHz, DMSO-d6) δ 8.4 (s, 1H), 7.29 (s, 1H), 3.97 (s, 3H); LC/MS: [(M+1)]⁺=223.

### Step B: 6-chloro-4-methoxynicotinonitrile

A solution of 5-bromo-2-chloro-4-methoxypyridine (5.0 g, 22.48 mmol) in DMF (80 mL) was purged nitrogen for 15 min. Next, Zn(CN)₂ (3.96 g, 33.7 mmol) and Pd(Ph₃P)₄ (2.60 g, 2.25 mmol) were added, successively. The resulting suspension was stirred at 95° C. for 12 h under nitrogen atmosphere. The reaction mixture was cooled to ambient temperature and filtered to remove inorganic solid. The solvent (DMF) was evaporated to provide the crude residue as an oil, which was purified on silica gel and eluted with 0-30% ethyl acetate/hexanes to afford the product. ¹HNMR (500 MHz, DMSO-d6) δ 8.69 (s, 1H), 7.50 (s, 1H), 4.04 (s, 3H); LC/MS: [(M+1)]⁺=169.

### Step C: 4-methoxy-6-vinylnicotinonitrile

A 20 mL microwave tube was charged with 6-chloro-4-methoxynicotinonitrile (200.0 mg, 1.2 mmol), bis(diphenylphosphino)ferrocene dichloropalladium (II), complex with dichloromethane (97.0 mg, 0.12 mmol), potassium vinyl trifluorobotate (318.0 mg, 2.37 mmol), triethylamine (0.33 mL, 2.37 mmol), and EtOH (6 mL). The microwave tube was evacuated and filled with nitrogen (two times) and heated to 140° C. After 1 h, the reaction mixture was diluted with water and extracted with EtOAc. The combined organic layers were washed with brine and dried over Na₂SO₄. The extracts were concentrated and chromatographed over a column of SiO₂ eluting with 0-30% EtOAc/hexanes. Evaporation of solvents yielded the 4-methoxy-6-vinylnicotinonitrile. ¹HNMR (500 MHz, DMSO-d6) δ 8.65 (s, 1H), 6.89 (s, 1H), 6.83 (dd, J=10.7 Hz, 1H), 6.42 (d, J=7.3 Hz, 1H), 5.70 (d, J=10.6 Hz, 1H), 4.05 (s, 3H); LC/MS: [(M+1)]⁺=161.

### Step D: 6-(2-bromo-1-hydroxyethyl)-4-methoxynicotinonitrile

A solution of 4-methoxy-6-vinylnicotinonitrile (80.0 mg, 0.499 mmol) in 1,4-dioxane (8 mL) and H₂O (4 mL) was treated with N-bromosuccinimide (89.0 mg, 0.499 mmol, 1.0 eq). The reaction mixture was allowed to stir at room temperature overnight. The reaction mixture was poured into H₂O (8 mL) and extracted with EtOAc (3×30 mL). The combined organic layers were washed with saturated aqueous NaCl (1×30 mL), dried over anhydrous Na₂SO₄. Evaporation of the solvent gave an oil that was purified over SiO₂ by eluting with 0-30% EtOAc/hexanes to afford 6-(2-bromo-1-hydroxyethyl)-4-methoxynicotinonitrile. ¹HNMR (500 MHz, DMSO-d6) δ 8.65 (s, 1H), 7.19 (s, 1H), 5.05 (t, J=5.4 Hz, 1H), 4.05 (s, 3H), 3.85 (dd, J=4.5 Hz, 1H), 3.75 (dd, J=6.1 Hz, 1H); LC/MS: [(M+1)]⁺=241.

### Step E: (4-methoxy-6-(oxiran-2-yl)nicotinonitrile

A solution of 6-(2-bromo-1-hydroxyethyl)-4-methoxynicotinonitrile (74.0 mg, 0.288 mmol) in anhydrous methanol (7 ml) with treated with sodium carbonate (61.0 mg, 0.576 mmol, 2.0 eq), and allowed to stir at room temperature overnight. The solvent was evaporated. The residue was taken up in EtOAc (30 mL) and washed with water and brine. After drying over Na₂SO₄, the organic layer was removed and the residue was purified over SiO₂ eluting with 10-45% EtOAc/hexanes to yield the title compound.

¹HNMR (500 MHz, DMSO-d6) δ 8.64 (s, 1H), 6.87 (s, 1H), 4.08 (dd, J=2.6 Hz, J=2.3 Hz, 1H), 4.03 (s, 3H), 3.26 (dd, J=4.6 Hz, J=5.4 Hz, 1H), 2.87 (dd, J=2.2 Hz, J=2.4 Hz, 1H); LC/MS: [(M+1)]⁺=177. Resolution of the epoxide was carried out (prep SFC, 160 mL/min., 10% MeOH in SC CO₂, AD-H) to provide:

(S)-4-Methoxy-6-(oxiran-2-yl)nicotinonitrile (fast eluting isomer A): LC/MS: [(M+1)]⁺=177.
(R)-4-Methoxy-6-(oxiran-2-yl)nicotinonitrile (slow eluting isomer B): LC/MS: [(M+1)]⁺=177.
Absolute chemistry was determined by using vibrational circular dichroism (VCD) spectroscopy with high confidence. Analysis was done comparing experimental data to the calculated VCD and IR spectra of the (R) and (S) compounds.

### Intermediate 7

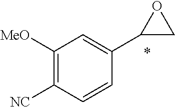 [SMILES: COc1cc(C2CO2)ccc1C#N]

### Step A: 4-formyl-2-methoxyphenyl trifluoromethanesulfonate

Potassium carbonate (36 g, 263 mmol) and 4-nitrophenyl trifluoromethanesulfonate (54.0 g, 197 mmol) was added to a solution of vanillin (20.0 g, 131 mmol) in DMF (200 mL) at rt and the reaction mixture was stirred for 8 h. EtOAc (600 mL) was added to the reaction mixture and the organic layer washed three times with water, dried, filtered, and concentrated. The crude compound was then purified by flash chromatography (10-30% ethylacetate/hexanes) to provide 4-formyl-2-methoxyphenyl trifluoromethanesulfonate. LC/MS: [(M+1)]⁺=285.

### Step B: 4-formyl-2-methoxybenzonitrile

A mixture of 4-formyl-2-methoxyphenyl trifluoromethanesulfonate (37.0 g, 130 mmol), zinc cyanide (61.0 g, 521 mmol) and tetrakis triphenylphosphine palladium (0) (22.6 g, 19.5 mmol) in DMF (300 mL) was stirred at 110° C. for 8 h. EtOAc was added to the reaction mixture and the organic layer was washed two times with water, dried, filtered and concentrated. The crude product was then purified by column chromatography eluting with 30% EtOAc/hexanes, which afforded 4-formyl-2-methoxybenzonitrile. LC/MS: [(M+1)]⁺=162.

### Step C: 2-methoxy-4-(oxiran-2-yl)benzonitrile

To a cool solution of NaH (0.16 g, 3.9 mmol) in THF (40 mL) was added dropwise a solution of trimethylsulfonium iodide (0.91 g, 4.5 mmol) in DMSO (20 mL). The resulting mixture was stirred at 0° C. under N₂ for 20 min. The solution of 4-formyl-2-methoxybenzonitrile (0.60 g, 3.7 mmol) in THF (20 mL) was added. The resulting reaction mixture was stirred at 0° C. under N₂ for 1 h, and then it was warmed gradually to room temperature and stirred at that temperature for 12 h. After the starting material was consumed as indicated by TLC (25% ethyl acetate/hexanes), the reaction mixture was cooled to 0° C. and quenched by the dropwise addition of water. The mixture was extracted with ethyl acetate (2×70 mL). The combined organic layers were washed with water, brine, then dried (MgSO₄) and filtered. The filtrates were concentrated in vacuo. The residue was purified by column chromatography (10-30% EtOAc/hexanes) to afford 2-methoxy-4-(oxiran-2-yl)benzonitrile. ¹H-NMR (500 MHz, CDCl₃) δ 7.57 (d, J=8 Hz, 1H), 6.99 (dd, J=1.1 Hz, J=1.2 Hz, 1H), 6.89 (s, 1H), 3.97 (s, 3H), 3.93 (m, 1H), 3.22 (dd, J=5.2 Hz, J=4.1 Hz, 1H), 2.77 (J=2.5 Hz, 1H); LC/MS: [(M+1)]⁺=176. Resolution of the epoxide was carried out on prep SFC in similar fashion to that described for INTERMEDIATE 2A and 2B to provide fast eluted 7A and slow eluted 7B.

### Intermediate 8

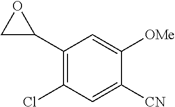 [SMILES: COc1cc(C2CO2)c(Cl)cc1C#N]

### Step A: di-t-butyl 2-(2-chloro-4-cyano-5 fluorophenyl)malonate

To sodium hydride (60% in mineral oil, 3.75 g, 94 mmol) under nitrogen was added dry DMF (150 mL) and the suspension was cooled in an ice bath. Di-t-butyl malonate (8.1 g, 37.5 mmol) was added dropwise over 15 minutes via syringe with hydrogen evolution. The suspension was stirred for 30 minutes after which time 5-chloro-2,4-difluorobenzonitrile (5.0 g, 28.8 mmol) in DMF (10 mL) was added dropwise over 15 minutes and the reaction was heated to 80° C. for 12 h. The reaction was cooled to room temperature, diluted with ether and quenched with aqueous ammonium chloride. The mixture was then extracted twice with ethyl acetate and the organic layers were washed with brine, dried over sodium sulfate and concentrated in vacuo. The residue was purified on silica gel (eluting with 2-10% ethyl acetate/hexanes) to give the title compound.

### Step B: methyl 2-(2-chloro-4-cyano-5-fluorophenyl)acetate

A solution of di-t-butyl 2-(2-chloro-4-cyano-5-fluorophenyl)malonate (9.10 g, 24.6 mmol) in 1:2 TFA: dichloromethane (25:50 mL) was stirred at RT for 3 hours and then concentrated in vacuo to give a solid after twice evaporating with toluene. The resulting solid was taken up in 1:1 methanol: dichloromethane (50 mL) and 2M trimethylsilyldiazomethane in ether was added until the yellow color persisted. Excess diazomethane was quenched with acetic acid and the mixture was concentrated. The residue was purified by flash chromatography (5-15% ethyl acetate/hexanes containing 5% DCM for solubility) to give separation from higher R_{f} 4-chloro-2-cyano-5-fluorophenyl isomer and still impure title isomer. Flash chromatography was repeated (50-100% DCM/hexanes) to afford the title product.

### Step C: methyl 2-(2-chloro-4-cyano-5-methoxyphenyl)acetate

A solution of methyl 2-(2-chloro-4-cyano-5-fluorophenyl)acetate (1.40 g, 6.15 mmol) in methanol (30 ml) was divided into two 20 mL microwave vials. Potassium carbonate (2×850 mg) was added to each vial. Each was heated in a microwave at 130° C. for 60 minutes, at which time HPLC/MS indicated no starting material was left, and the product was all hydrolyzed to the acid. Most of the methanol was removed in vacuo and the residue was diluted with water, acidified with 2M HCl and the mixture was extracted twice with ethyl acetate. The organic layers were washed with brine, dried over sodium sulfate and concentrated in vacuo. The crude product was taken up in 1:1 methanol: dichloromethane (50 mL), and the acid was re-esterified by the addition of 2M trimethylsilyldiazomethane in ether until a yellow color persisted. The excess diazomethane was quenched with acetic acid and the mixture was concentrated. Flash column chromatography (40-100% DCM/hexanes) gave the title compound.

### Step D: 5-chloro-4-(2-hydroxyethyl)-2-methoxybenzonitrile

To a solution of methyl 2-(2-chloro-4-cyano-5-methoxyphenyl)acetate (200 mg, 0.835 mmol) in THF (5 ml) was added 2M lithium borohydride (0.835 mL, 1.67 mmol) and the reaction was stirred at RT for 16 hours. The reaction was diluted with ether and quenched into water containing 2N HCl. The mixture was extracted twice with ethyl acetate and the organic layers were washed with brine, dried over sodium sulfate and concentrated in vacuo. The product mixture was separated by MPLC (20-60% ethylacetae/hexanes) to afford 5-chloro-4-(2-hydroxyethyl)-2-methoxybenzonitrile.

### Step E: 2-chloro-4-cyano-5-methoxyphenethyl methanesulfonate

A solution of 5-chloro-4-(2-hydroxyethyl)-2-methoxybenzonitrile (205 mg, 0.969 mmol), DIPEA (0.846 mL, 4.84 mmol) and pyridine (0.0780 ml, 0.969 mmol) in DCM (3 mL) was treated dropwise with mesyl chloride (0.110 mL, 1.42 mmol). The reaction was stirred for 2 hours and then diluted with DCM and washed twice with aqueous citric acid, then washed with brine, and dried over sodium sulfate and concentrated in vacuo. Purification of the residue by flash chromatography (20-50% ethyl acetate/hexanes) afforded 5-chloro-4-(2-hydroxyethyl)-2-methoxybenzonitrile.

### Step F: 5-chloro-2-methoxy-4-vinvylbenzonitrile

A solution of 2-chloro-4-cyano-5-methoxyphenethyl methanesulfonate (274 mg, 0.945 mmol) in DCM (4 mL) was treated with DBU (0.712 mL, 4.73 mmol) and stirred for 3 hours at 50° C., then at RT for 12 hours. TLC (50% ethyl acetate/hexanes) showed the complete conversion to a faster intense UV band for the product. The reaction was diluted with DCM and aqueous citric acid and the mixture was extracted twice with DCM. The organic layers were washed with brine, dried over sodium sulfate and concentrated in vacuo. Purification of the residue by flash chromatography (10-20% ethylacetate/hexanes) afforded 5-chloro-2-methoxy-4-vinylbenzonitrile.

### Step G: 5-chloro-2-methoxy-4-(oxiran-2-yl)benzonitrile

A solution of 5-chloro-2-methoxy-4-vinylbenzonitrile (130 mg, 0.671 mmol) in DCM (6 mL) was treated with 85% mCPBA (226 mg, 1.10 mmol) and stirred for 5 hours at RT when another portion of mCPBA (115 mg) was added. The reaction was stirred at room temperature for another 16 hours and was then diluted with DCM and stirred with saturated sodium bicarbonate containing some sodium bisulfite. The mixture was then extracted twice with DCM and the organic layers were washed with another portion of sodium bicarbonate and brine, dried over sodium sulfate and concentrated in vacuo to afford crude 5-chloro-2-methoxy-4-(oxiran-2-yl)benzonitrile. ¹H-NMR (500 MHz, CDCl₃) δ ppm 7.56 (s, 1H), 6.91 (s, 1H), 4.22 (dd, J=2.5, 3.9 Hz, 1H), 3.95 (s, 3H), 3.28 (dd, J=4.1, 5.5 Hz, 1H), 2.67 (dd, J=2.6, 5.8 Hz, 1H).

### Intermediate 9

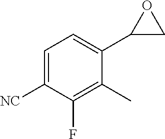

### Step A: 2-fluoro-3-methyl-4-vinylbenzonitrile

A mixture of 4-bromo-2-fluoro-3-methylbenzonitrile (7.0 g, 32.7 mmol), potassium vinyltrifluoroborate (5.3 g, 39.3 mmol), Pd(dppf)Cl₂ (0.5 g, 0.7 mmol) and TEA (30 mL) in EtOH (70 mL) was refluxed under Ar for 4 hours. After being cooled to room temperature, the reaction mixture was concentrated and the residue was purified by column chromatography (petrol ether:EtOAc=10:1) to afford 2-fluoro-3-methyl-4-vinylbenzonitrile as a white solid.

### Step B: 2-fluoro-3-methyl-4-(oxiran-2-yl)benzonitrile

A mixture of 2-fluoro-3-methyl-4-vinylbenzonitrile (4.6 g, 28.5 mmol) and mCPBA (85%, 12.3 g, 71.4 mmol) in 300 mL of DCM was stirred at room temperature for 120 hours. The reaction mixture was cooled to 0° C. and washed subsequently with saturated NaHCO₃ (50 mL), saturated Na₂SO₃ (50 mL), 5% NaOH (50 mL×2) and brine (50 mL), dried over anhydrous Na₂SO₄ and concentrated. The residue was purified by column chromatography (petrol ether:EtOAc=20:1) to afford 2-fluoro-3-methyl-4-(oxiran-2-yl)benzonitrile. ¹H-NMR (400 MHz, CDCl₃) δ ppm 7.36-7.39 (m, 1H), 7.04-7.06 (m, 1H), 3.92-3.94 (m, 1H), 3.15-3.17 (m, 1H), 2.57-2.59 (m, 1H), 2.30 (d, J=2.0 Hz, 3H).

### Intermediate 10

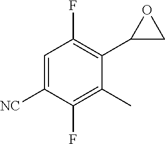

### Step A: 4-amino-2,5-difluorobenzonitrile

2, 4, 5-trifluorobenzonitrile (20 g, 127 mmol) was added to 15 mL of liquid NH₃ and was reacted under 1.4 MPa at 60° C. for 4 hours. The reaction mixture was then cooled to room temperature, diluted with ether (200 mL), washed with brine, dried and concentrated to give 4-amino-2,5-difluorobenzonitrile.

### Step B: 4-amino-3-bromo-2,5-difluorobenzonitrile

To a solution of 4-amino-2,5-difluorobanzonitrile (19 g, 0.13 mol) in 190 mL of AcOH and 8 mL of H₂O was added Br₂ (6.27 mL, 0.13 mol) dropwise. The resulting mixture was stirred at room temperature for 4 hours. The mixture was then poured into water, and the white precipitates were filtered, washed with water and dried to give 4-amino-3-bromo-2,5-difluorobenzonitrile.

### Step C: 4-amino-2,5-difluoro-3-methylbenzonitrile

A mixture of 4-amino-3-bromo-2,5-difluorobenzonitrile (15 g, 64 mmol), SnMe₄ (23 g, 128 mmol), LiCl (5.5 g, 128 mmol) and Pd(PPh₃)₄ (3.72 g, 3.2 mmol) in 300 mL of DMF was heated under N₂ at 90˜100° C. overnight. The mixture was then cooled to room temperature and diluted with 250 mL of EtOAc and filtered. The filtrates were washed with water and brine, dried over anhydrous Na₂SO₄ and concentrated to dryness. The residue was purified by column chromatography (DCM/PE=1/5) to give 4-amino-2,5-difluoro-3-methylbenzonitrile.

### Step D: 4-Bromo-2,5-difluoro-3-methylbenzonitrile

To a solution of 4-amino-2,5-difluoro-3-methylbenzonitrile (10.5 g, 62.4 mol) in 40 mL of concentrated HBr acid and 20 mL of water was added a solution of sodium nitrite (4.72 g, 68.6 mmol) in 10 mL of water at −5˜0° C. CuBr (18 g, 124 mol) in 40 mL concentrated HBr acid was added dropwise at room temperature. The light yellow precipitate produced was collected by filtration and was washed with concentrated hydrochloric acid and water then dried at 40-50° C. by vacuum to give the title compound.

### Step E: 2,5-Difluoro-3-methyl-4-vinylbenzonitrile

A mixture of 4-bromo-2,5-difluoro-3-methylbenzonitrile (4.0 g, 17.2 mmol), potassium vinyltrifluoroborate (2.5 g, 18.9 mmol) and Pd(dppf)₂Cl₂ (0.4 g, 0.6 mmol) in 40 mL of EtOH and 12 mL of TEA was refluxed under Ar for 4 hours. The reaction mixture was concentrated and the residue was purified by column chromatography (petrol ether:EtOAc=10:1) to afford the title compound as a white solid.

### Step F: 2,5-Difluoro-3-methyl-4-(oxiran-2-yl)benzonitrile

A mixture of 2,5-difluoro-3-methyl-4-vinylbenzonitrile (2.7 g, 15.1 mmol) and mCPBA (85%, 6.5 g, 35.1 mmol) in 270 mL of DCM was stirred at room temperature for 120 hours. The reaction mixture was cooled to 0° C. and was washed subsequently with saturated NaHCO₃ (50 mL), saturated Na₂SO₃ (50 mL), 5% NaOH (50 mL×2) and brine (50 mL), dried over anhydrous Na₂SO₄ and concentrated. The residue was purified by column chromatography (petrol ether:EtOAc=20:1) to afford the title compound.

### Intermediate 11

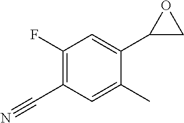

### Step A: 4-bromo-2-fluoro-5-methylaniline

To a solution of 2-fluoro-5-methylaniline (20 g, 0.16 mol) in DCM was added TBATB (118 g, 0.17 mol) at 0° C. portionwise, then the mixture was stirred at rt for 1 hour, water was added and extracted with EtOAc (200 mL×3). The combined organic layers were washed with water and brine, dried and concentrated. The residue was purified by column chromatography to afford 4-bromo-2-fluoro-5-methylaniline.

### Step B: 4-bromo-2-fluoro-5-methylbenzonitrile

A suspension of 4-bromo-2-fluoro-5-methylaniline (15 g, 73.5 mmol) in 30 mL of concentrated HCl was added 30 mL of water and a solution of NaNO₂ (5.33 g, 77.2 mmol) in water (20 mL) was added over a 20 minute period at 0° C. This diazonium solution was then brought to pH 6 with NaHCO₃. In a separate vial, a solution of CuSO₄ (22.9 g, 91.9 mmol) in water (100 mL) was added dropwise to a solution of KCN (23.9 mg, 368 mmol) in water (100 mL) at 0° C., then toluene (100 mL) was added and the mixture was stirred and heated to 60° C. The previously prepared diazonium solution was added dropwise to the brown CuCN solution at rt for 1 hour and EtOAc (100 ml) was added. The organic phase was washed with brine (200 mL) and concentrated. The crude product was purified via Prep-TLC to afford the 4-bromo-2-fluoro-5-methylbenzonitrile.

### Step C: 2-fluoro-5-methyl-4-vinylbenzonitrile

A mixture of 4-bromo-2-fluoro-5-methylbenzonitrile (4.0 g, 18.7 mmol), potassium vinyltrifluoroborate (2.8 g, 20.6 mmol) and Pd(dppf)₂Cl₂ (0.4 g, 0.6 mmol) in 40 mL of EtOH and 13 mL of TEA was refluxed under Ar for 4 hours. The reaction mixture was concentrated, and the residue was purified by column chromatography (petrol ether:EtOAc=10:1) to afford the title compound as a yellow solid.

### Step D: 2-fluoro-5-methyl-4-(oxiran-2-yl)benzonitrile

A mixture of 2-fluoro-5-methyl-4-vinylbenzonitrile (2.6 g, 16.1 mmol) and mCPBA (85%, 7 g, 40 mmol) in 300 mL of DCM was stirred at room temperature for 120 hours. The reaction mixture was cooled to 0° C. and was washed subsequently with saturated NaHCO₃ (50 ml), saturated Na₂SO₃ (50 mL), 5% NaOH (50 mL×2) and brine (50 mL), dried over anhydrous Na₂SO₄ and concentrated. The residue was purified by column chromatography (petrol ether:EtOAc=20:1) to afford the title compound.

### Intermediate 12

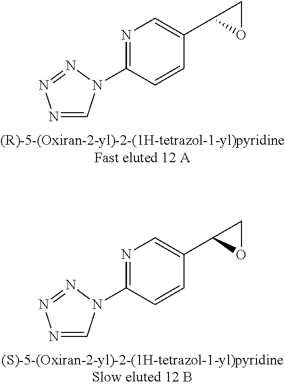 [SMILES: c1cc(-n2cnnn2)ncc1[C@H]1CO1 ; c1cc(-n2cnnn2)ncc1[C@@H]1CO1]

### Step A: 5-Bromo-2-(1H-tetrazol-1-yl)pyridine

To a solution of 5-bromopyridin-2-amine (5.0 g, 28.9 mmol) in acetic acid (40 ml, 699 mmol) was added (diethoxymethoxy) ethane (7.70 ml, 46.2 mmol), followed by sodium azide (2.82 g, 43.3 mmol). The mixture was heated at 80° C. for 1 h, cooled to room temperature and diluted with water. Precipitate was collected by filtration and dried under high vacuum to provide the title compound.

### Step B: 5-Ethenyl-2-(1H-tetrazol-1-yl)pyridine

To a stirring solution of 5-bromo-2-(1H-tetrazol-1-yl)pyridine (1.0 g, 4.42 mmol), in EtOH (70 mL) were added bis[(diphenylphosphino)ferrocene]dichloropalladium(II), complex with dichloromethane (0.361 g, 0.442 mmol), potassium vinyl trifluoroborate (1.18 g, 8.85 mmol, 2 equiv.), triethylamine (1.23 mL, 8.85 mmol, 2 equiv), and water (0.5 mL). The reaction mixture was heated at reflux (90° C., oil bath) under N₂. Upon completion (1-2 h) as determined by reverse phase HPLC-MS and TLC (eluent: 10% ethyl acetate in hexane), the mixture was cooled to room temperature, diluted with water. The organic layer was separated, and the aqueous was extracted with EtOAc. The combined organic layers were washed with brine, dried over MgSO₄, and concentrated. The crude material was chromatographed over a column of SiO₂ (0-20% EtOAc in hexane as eluent). Evaporation of the solvent yielded the title compound. LCMS [M+1]⁺=174.0.

### Step C: 5-(Oxiran-2-yl)-2-(1H-tetrazol-1-yl)pyridine

To a solution of 5-ethenyl-2-(1H-tetrazol-1-yl)pyridine (0.664 g, 3.83 mmol) in a 2:1 ratio of H₂O:t-BuOH (30 mL) was added N-bromosuccinimide (0.751 g, 4.22 mmol) in portions over 5 min. The mixture was heated at 40° C. for 1 h, cooled to 5° C., made basic with sodium hydroxide aqueous solution (0.46 g in 5 mL of H₂O, 11.50 mmol), stirred for another 1 h at the same temperature, and poured into H₂O (10 mL). The product was precipitated out as white solid. The solid was collected by filtration, washed with water, and dried in vacuum. ¹H NMR (500 MHz, DMSO-d6) δ 10.17 (s, 1H), 8.60 (d, J=1.4 Hz, 1H), 8.04-7.99 (m, 2H), 4.14 (dd, J=2.7 Hz, J=2.8 Hz, 1H), 3.23 (t, J=4.6 Hz, 1H), 3.02 (dd, J=25 Hz, 1H); LCMS [M+1]⁺=190. Further chiral separation (AD-H 30×250 mm, 50% MeOH/CO₂, 70 mL/min, 100 bar, 46 mg in MeOH/DCM) conducted by the separation and purification group afforded fast eluted 12A (R)-5-(oxiran-2-yl)-2-1H-tetrazol-1-yl)pyridine and slow eluted 12B (S)-5-(oxiran-2-yl)-2-(1H-tetrazol-1-yl)pyridine. Absolute chemistry was determined by using VCD spectroscopy with high confidence. Analysis was done comparing experimental data to the calculated VCD and IR spectra of the (R) and (S) compounds.

### Intermediate 13

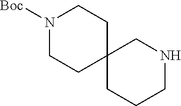 [SMILES: CC(C)(C)OC(=O)N1CCC2(CCCNC2)CC1]

Step A: tert-butyl 4-(hydroxymethyl)piperidine-1-carboxylate

The mixture of 70 g of LiAlH₄ in 1500 mL of THF was cooled to 0° C., then 180 g of 1-tert-butyl 4-methyl piperidine-1,4-dicarboxylate in THF was added dropwise. When the reaction was finished, 200 mL of ethyl acetate and solid anhydrous Na₂SO₄ were added. Water was added until solution became clear. The mixture was filtered and the filtrates were evaporated to afford the title compound.

### Step B: tert-butyl 4-formylpiperidine-1-carboxylate

The solution of 200 mL of DMSO in CH₂Cl₂ was cooled to −78° C., and 118 mL of (COCl)₂ was added dropwise. Then 255 g of tert-butyl 4-(hydroxymethyl)piperidine-1-carboxylate was also added dropwise. The mixture was stirred for 4 hours. After the reaction was finished, 638 mL of Et₃N was added at −78° C. The organic layer was washed by brine, dried and purified by column chromatography to afford tert-butyl 4-formylpiperidine-1-carboxylate.

### Step C: tert-butyl 4-(2-cyanoethyl)-4-formylpiperidine-1-carboxylate

tert-Butyl 4-formylpiperidine-1-carboxylate was dissolved in 66 mL of acrylonitrile, and 5 g of 50% aqueous sodium hydroxide solution was added. The reaction was heated at 50° C. until TLC showed it was finished. The mixture was then poured into 700 mL of ether. The organic layer was separated, and washed with brine. The crude product was purified with column chromatography to afford tert-butyl 4-(2-cyanoethyl)-4-formylpiperidine-1-carboxylate.

### Step D: tert-butyl 2,9-diazaspiro[5.5]undecane-9-cane-9-carboxylate

30 g of tert-butyl 4-(2-cyanoethyl)-4-formylpiperidine-1-carboxylate was dissolved in methanol saturated with ammonia, and 15 g of Raney Ni was added. The reaction mixture was heated to 110° C. and kept at 80 atmosphere in a 2 L high-pressure autoclave. After the reaction was finished, the mixture was cooled to room temperature, filtered to remove the catalyst and the filtrates were concentrated to give a residue, which was purified by column chromatography to afford tert-butyl 2,9-diazaspiro[5.5]undecane-9-carboxylate.

### Intermediate 14

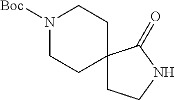 [SMILES: CC(C)(C)OC(=O)N1CCC2(CCNC2=O)CC1]

tert-Butyl 1-oxo-2,8-diazaspiro[4.5]decane-8-carboxylate is commercially available from a number of vendors, for example, Shanghai AQ BioPharma Co., Ltd, catalog #ABP1882. Alternatively, it may be prepared in various ways, including the procedure described below:

### Step A: 1-tert-butyl 4-methyl 4-(cyanomethyl)piperidine-1,4-dicarboxylate

To a solution of commercially available 1-tert-butyl 4-methyl piperidine-1,4-dicarboxylate (200 g, 0.82 mol) in anhydrous THF (2 L) was added LDA (2M in THF, 575 mL, 1.15 mol) dropwise at −65° C. under N₂. The mixture was stirred at −65° C. for 1.5 h. To the mixture was added bromoacetonitrile (148 g, 1.23 mol) in anhydrous THF (500 mL) at −65° C. The mixture was stirred at −65° C. for 1 h, then warmed up to room temperature and stirred overnight. The reaction was quenched with water (800 mL) at 0° C. and the combined reaction mixture was concentrated under vacuum to give a crude product, which was extracted with ethyl acetate (1 L three times). The combined organic phases were washed with brine (1 L) and dried over Na₂SO₄. The organic layer was filtered and the filtrate was concentrated under vacuum to give a crude product, which was purified by column chromatography on silica gel eluting with petroleum ether/ethyl acetate (from petroleum ether to 2/1) to give title compound. ¹H-NMR (400 MHz, CDCl₃) δ 3.900-3.750 (m, 5H), 3.120-3.000 (m, 2H), 2.612-2.562 (m, 2H), 2.190-2.111 (m, 2H), 1.590-1.502 (m, 2H), 1.402 (s, 9H).

### Step B: tert-butyl 1-oxo-2,8-diazaspiro[4.5]decane-8-carboxylate

A suspension of 1-tert-butyl 4-methyl 4-(cyanomethyl)piperidine-1,4-dicarboxylate (70.0 g, 247.9 mmol) and Raney Ni (60 g) in MeOH (1500 mL) and NH₃.H₂O (80 mL) was stirred at 2 MPa of hydrogen at 50° C. for 18 h. The reaction mixture was filtered through a pad of CELITE® and the filtrate was concentrated under vacuum to give a crude product, which was washed with ethyl acetate (200 mL) to give title compound. ¹H-NMR (400 MHz, CDCl₃) δ 6.05 (s, 1H), 4.0 (s, 2H), 3.37-3.34 (m, 2H), 3.02-2.96 (m, 2H), 2.08-2.05 (m, 2H), 1.88-1.87 (m, 2H), 1.51-1.41 (m, 11H).

### Intermediate 15

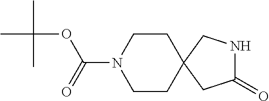 [SMILES: CC(C)(C)OC(=O)N1CCC2(CC1)CNC(=O)C2]

### Step A: tert-Butyl 4-(2-ethoxy-2-oxoethylidene)piperidine-1-carboxylate

Into a 10-L 4-necked round-bottom flask purged and maintained with an inert atmosphere of nitrogen was placed a suspension of NaH (74.0 g, 2.16 mol 1.05 equiv, 70%) in tetrahydrofuran (2000 mL) at 0° C., then added dropwise ethyl 2-(diethoxyphosphoryl)acetate (514 g, 2.06 mol, 1.05 equiv, 98%) with stirring at 0° C. This was followed by the dropwise addition of a solution of tert-butyl 4-oxopiperidine-1-carboxylate (400 g, 1.97 mol, 1.00 equiv, 98%) in tetrahydrofuran (1200 mL) dropwise with stirring at 0° C. The resulting solution was stirred for 60 min at room temperature, then was quenched by the addition of water (2000 mL). The resulting solution was extracted with ethyl acetate (2×1000 mL). The organic layers were combined, dried over anhydrous magnesium sulfate and concentrated under vacuum. The residue was washed with hexane (1000 mL) and dried. This resulted in tert-butyl 4-(2-ethoxy-2-oxoethylidene)piperidine-1-carboxylate.

### Step B: tert-butyl 4-(2-ethoxy-2-oxoethyl)-4-(nitromethyl)piperidine-1-carboxylate

Into a 3000-mL 4-necked round-bottom flask were placed potassium carbonate (93.2 g, 662 mmol, 0.50 equiv) and DMSO (2000 mL). The resulting solution was heated to 80° C. This was followed by the addition of tert-butyl 4-(2-ethoxy-2-oxoethylidene)piperidine-1-carboxylate (368 g, 1.30 mol, 1.00 equiv, 95%) and CH₃NO₂ (417 g, 6.70 mol, 5.00 equiv, 98%) slowly. The resulting solution was stirred for 120 min at 90° C. After cooled to room temperature, the reaction mixture was adjusted to pH 5 with HCl (0.5 mol/L) and diluted with water (2000 mL). The resulting solution was extracted with ether (3×1500 mL). The organic layers were combined, washed with water (2000 mL) and brine (2000 mL), dried and concentrated under vacuum. The residue was applied onto a silica gel column and eluted with ethyl acetate/petroleum ether (1:20˜1:15˜1:10) to afford the title compound.

### Step C: 3-oxo-2,8-diaza-spiro[4,5]decane-8-carboxylic acid tert-butylester

A mixture of tert-butyl 4-(2-ethoxy-2-oxoethyl)-4-(nitromethyl)piperidine-1-carboxylate (330 g, 990 mmol, 1.00 equiv, 99%) and Ni (40 g, 0.15 equiv) in ethanol (1200 mL) was stirred for 24 h under a hydrogen atmosphere at rt. The solid was filtered out. The filtrate was concentrated under vacuum. The crude product was purified by re-crystallization from ether to afford the title compound. LC-MS (ES, m/z): 199 [M+H]⁺; ¹H NMR (400 MHz, CDCl3, ppm): 1.447-1.476 (9H, s), 1.597-1.673 (4H, m, J=30.4 Hz), 2.235 (2H, s), 3.226 (2H, s), 3.284-3.348 (2H, m, J=25.6 Hz), 3.507-3.567 (2H, m, J=24 Hz), 6.048 (1H, s).

### Intermediate 16

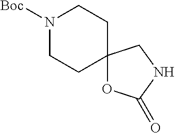 [SMILES: CC(C)(C)OC(=O)N1CCC2(CC1)CNC(=O)O2]

### Step A: tert-butyl 4-(2-ethoxy-2-oxoethyl)-4-hydroxypiperidine-1-carboxylate

To a solution of lithium bis(trimethylsilyl)amide (120 mL, 1.0 M solution in THF, 0.12 mol) in THF (120 mL) at −78° C. was added ethyl acetate (13 mL). Next, a solution of tert-butyl 4-oxopiperidine-1-carboxylate (20 g, 0.1 mol) in THF (80 mL) was added at −78° C. After the addition, the mixture was warmed up to 0° C. and stirred for another 2 h. The aqueous layer was extracted with ethyl acetate; the organic phase was washed with brine, dried over Na₂SO₄ and concentrated to afford the crude tert-butyl 4-(2-ethoxy-2-oxoethyl)-4-hydroxypiperidine-1-carboxylate.

### Step B: 2-(1-(tert-butoxycarbonyl)-4-hydroxypiperidin-4-yl)acetic acid

A solution of tert-butyl 4-(2-ethoxy-2-oxoethyl)-4-hydroxypiperidine-1-carboxylate (30.0 g, 0.105 mol) in methanol (130 mL) and 2N NaOH solution (100 mL, 0.2 mol) was stirred at 25° C. for 1.5 h, then the mixture was evaporated and the aqueous layer was extracted with ethyl acetate. The water phase was adjusted to pH 6 with 2N HCl, the aqueous layer was extracted with ethyl acetate, then the organic phase was washed with brine, dried over Na₂SO₄ and concentrated to afford 2-(1-(tert-butoxycarbonyl)-4-hydroxypiperidin-4-yl)acetic acid.

### Step C: tert-butyl 2-oxo-1-oxa-3,8-diazaspiro[4.5]decane-8-carboxylate

A mixture of 2-(1-(tert-butoxycarbonyl)-4-hydroxypiperidin-4-yl)acetic acid (22 g, 0.085 mol), DPPA (30 g, 0.11 mol), Et₃N (150 mL) in toluene (400 mL) was stirred at 105° C. under nitrogen for 12 h. The reaction mixture was quenched by the addition of the saturated aqueous NaHCO₃, the organic phase was washed with brine, dried over Na₂SO₄, the mixture was concentrated to remove most of toluene, then ether was added and filtered. The filter cake was washed with ether, the solid was dried under vacuum to afford the title compound. ¹H NMR (300 MHz, CDCl₃) δ: 5.35 (brs, 1H), 3.83-3.85 (m, 2H), 3.26-3.35 (m, 4H), 1.93-1.97 (m, 2H), 1.68-1.75 (m, 2H), 1.46 (s, 9H).

### Intermediate 17

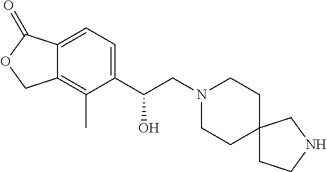 [SMILES: Cc1c([C@@H](O)CN2CCC3(CCNC3)CC2)ccc2c1COC2=O]

### (R)-5-(1-Hydroxy-2-(2,8-diazaspiro[4.5]decan-8-yl)ethyl)-4-methylisobenzofuran-1 (3H)-one

4-Methyl-5-[(2R)-oxiran-2-yl]-2-benzofuran-1(3H)-one (INTERMEDIATE 1B) (2.0 g, 10.52 mmoles) and tert-butyl 2,8-diazaspiro[4.5]decane-2-carboxylate (2.53 g, 10.52 mmoles) were added to a 20 ml microwave vial. Ethanol (15 ml) was then added. The vial was capped and the mixture was irradiated at 150° C. for 70 min in a Biotage microwave reactor. The ethanol was then removed in vacuo to give the crude product, to which was then added 4 M hydrochloric acid in 1,4-dioxanes (20 mL). The mixture was stirred at room temperature for 1 h. The mixture was then concentrated in vacuo to give the title compound, which was used in the next step without further purification. LC-MS (IE, m/z): 331 [M+1]⁺.

### Intermediate 18

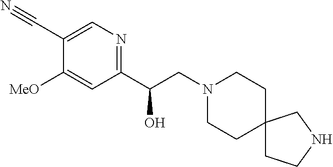 [SMILES: COc1cc([C@H](O)CN2CCC3(CCNC3)CC2)ncc1C#N]

### Step A: (R)-tert-butyl 8-(2-(5-cyano-4-methoxypyridin-2-yl)-2-hydroxyethyl)-2,8-diazaspiro[4.5]decane-2-carboxylate

A microwave tube was charged with tert-butyl 2,8-diazaspiro[4.5]decane-2-carboxylate (0.500 g, 2.08 mmol), (S)-4-methoxy-6-(oxiran-2-yl)nicotinonitrile (INTERMEDIATE 6A) (0.367 g, 2.08 mmol), and ethanol (4.0 mL). The solution was degassed and filled with nitrogen (3×), then sealed and heated in a microwave reactor to 140° C. for 1 h. The reaction was cooled to room temperature and concentrated in vacuo. The resulting residue was purified by prep TLC (2% MeOH:DCM) to provide the title compound. LC-MS (IE, m/z): 417 [M+H]; ¹H NMR (500 MHz, CDCl₃): 8.44 (s, 1H), 7.21 (s, 1H), 4.71 (m, 1H), 3.95 (s, 3H), 3.28 (m, 2H), 3.09 (m, 2H), 2.78 (m, 1H), 2.65 (m, 1H), 2.48 (m, 1H), 2.37 (m, 3H), 1.58 (m, 4H) 1.38 (s, 9H).

### Step B: (R)-6-(1-hydroxy-2-(2,8-diazaspiro[4.5]decan-8-yl)ethyl)-4-methoxynicotinonitrile

To a solution of (R)-tert-butyl 8-(2-(5-cyano-4-methoxypyridin-2-yl)-2-hydroxyethyl)-2,8-diazaspiro[4.5]decane-2-carboxylate (0.500 mg, 1.20 mmol) in dichloromethane (4.0 mL) at 0° C. was added trifloruoacetic acid (2.0 mL). The reaction was stirred at room temperature for 30 min. The mixture was concentrated and the resulting residue was partitioned between DCM and saturated sodium bicarbonate solution which was adjusted with 1 N NaOH to maintain pH˜9. The aqueous layer was extracted with iPrOH:CHCl₃ (1:3, 3×) and the combined organic layers were washed with brine, dried over Mg₂SO₄, filtered and concentrated to provide (R)-6-(1-hydroxy-2-(2,8-diazaspiro[4.5]decan-8-yl)ethyl)-4-methoxynicotinonitrile. LC-MS (IE, m/z): 317 [M+1]⁺.

### Intermediate 19

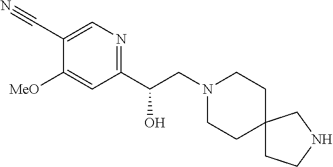 [SMILES: COc1cc([C@@H](O)CN2CCC3(CCNC3)CC2)ncc1C#N]

### Step A: (S)-tert-butyl 8-(2-(5-cyano-4-methoxypyridin-2-yl)-2-hydroxyethyl)-2,8-diazaspiro[4.5]decane-2-carboxylate

(S)-tert-Butyl 8-(2-(5-cyano-4-methoxypyridin-2-yl)-2-hydroxyethyl)-2,8-diazaspiro[4.5]decane-2-carboxylate was prepared in a similar fashion to that described for the synthesis of (R)-tert-butyl 8-(2-(5-cyano-4-methoxypyridin-2-yl)-2-hydroxyethyl)-2,8-diazaspiro[4.5]decane-2-carboxylate (INTERMEDIATE 18, Step A) from tert-butyl 2,8-diazaspiro[4.5]decane-2-carboxylate and (R)-4-methoxy-6-(oxiran-2-yl)nicotinonitrile (INTERMEDIATE 6 B)

### Step B: (S)-6-(1-hydroxy-2-(2,8-diazaspiro[4.5]decan-8-yl)ethyl)-4-methoxynicotinonitrile

(S)-6-(1-Hydroxy-2-(2,8-diazaspiro[4.5]decan-8-yl)ethyl)-4-methoxynicotinonitrile was prepared in a similar fashion to that described for the synthesis of (R)-6-(1-hydroxy-2-(2,8-diazaspiro[4.5]decan-8-yl)ethyl)-4-methoxynicotinonitrile (INTERMEDIATE 18, Step B). LC-MS (IE, m/z): 317 [M+1]⁺.

### Intermediate 20

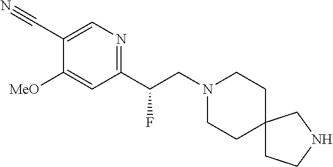 [SMILES: COc1cc([C@@H](F)CN2CCC3(CCNC3)CC2)ncc1C#N]

### Step A: (S)-tert-butyl 8-(2-(5-cyano-4-methoxypyridin-2-yl)-2-fluoroethyl)-2,8-diazaspiro[4.5]decane-2-carboxylate

To a solution of (R)-tert-butyl 8-(2-(5-cyano-4-methoxypyridin-2-yl)-2-hydroxyethyl)-2, 8-diazaspiro[4.5]decane-2-carboxylate (INTERMEDIATE 18, Step A) (340 mg, 0.816 mmol) and triethylamine (114 μl, 0.816 mmol) in THF (4.0 mL) was added DAST (129 μl, 0.980 mmol) at room temperature in a plastic vial. The mixture was stirred at room temperature for 45 min. The reaction was quenched with water. After concentration, the residue was partitioned between EtOAc and water. The aqueous layer was extracted with EtOAc (2×). The combined organic phase was washed with brine, dried over anhydrous MgSO₄, and filtered. After concentration, the mixture was purified by prep TLC (silica gel, 10% MeOH/DCM) to provide (R)-tert-butyl 8-(2-(5-cyano-4-methoxypyridin-2-yl)-2-fluoroethyl)-2, 8-diazaspiro[4.5]decane-2-carboxylate. LC-MS (IE, m/z): 419 [M+1]⁺.

### Step B: (S)-6-(1-fluoro-2-(2,8-diazaspiro[4.5]decan-8-yl)ethyl)-4-methoxynicotinonitrile

(S)-6-(1-Fluoro-2-(2,8-diazaspiro[4.5]decan-8-yl)ethyl)-4-methoxynicotinonitrile was prepared in a similar fashion to that described for the synthesis of INTERMEDIATE 18 Step B starting from (S)-tert-butyl 8-(2-(5-cyano-4-methoxypyridin-2-yl)-2-fluoroethyl)-2,8-diazaspiro[4.5]decane-2-carboxylate. LC-MS (IE, m/z): 319 [M+1]⁺.

### Intermediate 21

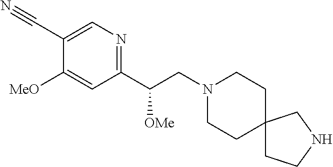 [SMILES: COc1cc([C@H](CN2CCC3(CCNC3)CC2)OC)ncc1C#N]

### Step A: (S)-tert-butyl 8-(2-(5-cyano-4-methoxypyridin-2-yl)-2-methoxyeth-2,8-diazaspiro[4.5]decane-2-carboxylate

To a solution of (S)-tert-butyl 8-(2-(5-cyano-4-methoxypyridin-2-yl)-2-hydroxyethyl)-2, 8-diazaspiro[4.5]decane-2-carboxylate (INTERMEDIATE 19, Step A) (70 mg, 0.168 mmol) in DMF (1 mL) at 0° C. was added KHMDS (0.252 mL, 0.252 mmol, 1 M in toluene). After 30 min, MeI (10.5 μL, 0.168 mmol) was added and the mixture was stirred for 2 h, warming slowly to rt. The reaction was quenched with saturated NH₄Cl and extracted with EtOAc (3×). The combined organic layers were washed with water, and brine, dried (Na₂SO₄), filtered and concentrated. The reaction mixture was purified by prep TLC (5% MeOH in DCM) to provide the title compound. LC-MS (IE, m/z): 431 [M+1]⁺.

### Step B: (S)-4-Methoxy-6-(1-methoxy-2-(2,8-diazaspiro[4.5]decan-8-yl)ethyl)nicotinonitrile

The title compound was prepared in a similar fashion to that described for the synthesis of INTERMEDIATE 18 Step B starting from (S)-tert-butyl 8-(2-(5-cyano-4-methoxypyridin-2-yl)-2-methoxyethyl)-2,8-diazaspiro[4.5]decane-2-carboxylate.

### Intermediate 22

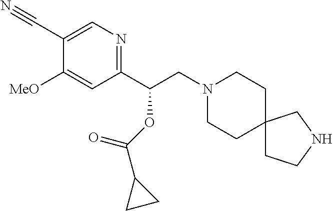 [SMILES: COc1cc([C@H](CN2CCC3(CCNC3)CC2)OC(=O)C2CC2)ncc1C#N]

### Step A: (S)-tert-butyl 8-(2-(5-cyano-4-methoxypyridin-2-yl)-2-((cyclopropanecarbonyl)oxy) ethyl)-2, 8-diazaspiro[4.5]decane-2-carboxylate

To a solution of (S)-tert-butyl 8-(2-(5-cyano-4-methoxypyridin-2-yl)-2-hydroxyethyl)-2,8-diazaspiro[4.5]decane-2-carboxylate (INTERMEDIATE 19, Step A) (80 mg, 0.192 mmol) in pyridine (1 ml) at rt was added cyclopropanecarbonyl chloride (60.2 mg, 0.576 mmol) and the mixture was stirred for 8 h. The mixture was poured into water and extracted with EtOAc (3×). The combined organic layers were washed with water (2×) and brine, then dried (Na₂SO₄), filtered and concentrated. The residue was purified by prep TLC (5% MeOH in DCM) to provide the title compound. LC-MS (IE, m/z): 485 [M+1]⁺.

### Step B: (S)-1-(5-cyano-4-methoxypyridin-2-yl)-2-(2,8-diazaspiro[4.5]decan-8-yl)ethyl cyclopropanecarboxylate

The title compound was prepared in a similar fashion to that described for the synthesis of INTERMEDIATE 18 Step B starting from (S)-tert-butyl 8-(2-(5-cyano-4-methoxypyridin-2-yl)-2-((cyclopropanecarbonyl)oxy)ethyl)-2,8-diazaspiro[4.5]decane-2-carboxylate. LC-MS (IE, m/z): 385 [M+1]⁺.

### Intermediate 23

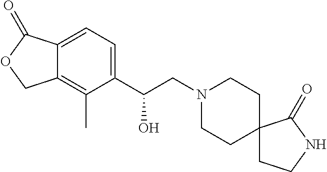 [SMILES: Cc1c([C@@H](O)CN2CCC3(CCNC3=O)CC2)ccc2c1COC2=O]

### Step A: 2,8-diazaspiro[4.5]decan-1-one hydrochloride

To a solution of tert-butyl 1-oxo-2,8-diazaspiro[4.5]decane-8-carboxylate (INTERMEDIATE 14) (92 g, 0.36 mol) in CH₂Cl₂ (1 L) was slowly added a 4 M HCl solution in EtOAc (500 mL). The mixture was stirred for 8 h at rt. The mixture was concentrated under vacuum to afford the title compound. ¹H-NMR (400 MHz, DMSO-d6): δ 9.35 (s, 1H), 9.02 (s, 1H), 7.72 (s, 1H), 3.30-3.20 (m, 2H), 3.16 (m, J=6.8 Hz, 2H), 2.98-2.85 (m, 2H), 1.96 (m, J=6.8 Hz, 2H), 1.90-1.80 (m, 2H), 1.55 (d, J=14 Hz, 2H).

### Step B: (R)-8-(2-hydroxy-2-(4-methyl-1-oxo-1,3-dihydroisobenzofuran-5-yl)ethyl)-2,8-diazaspiro[4.5]decan-1-one

To a solution of 2,8-diazaspiro[4.5]decan-1-one hydrochloride (68 g, 0.35 mol) in ethanol (1.5 L) was added Et₃N (55 mL). The mixture was stirred for 2 hours. Next, (R)-4-methyl-5-(oxiran-2-yl)isobenzofuran-1(3H)-one (65 g, 0.34 mol) was added. The mixture was heated to reflux for 40 h. After filtration, the solid was collected to provide the title compound. The filtrate was concentrated and purified by SFC separation to provide additional (R)-8-(2-hydroxy-2-(4-methyl-1-oxo-1,3-dihydroisobenzofuran-5-yl)ethyl)-2,8-diazaspiro[4.5]decan-1-one. ¹H-NMR (400 Hz, CDCl3): δ 7.82-7.75 (m, 2H), 6.00 (s, 1H), 5.24 (s, 2H), 5.08 (dd, J=2.1 Hz and 10.4 Hz, 1H), 4.21 (s, 1H), 3.35 (t, J=6.8 Hz, 2H), 3.17-3.14 (m, 1H), 2.85-2.82 (m, 1H), 2.57 (dd, J=2.1 Hz and 10.4 Hz, 1H), 2.49 (t, J=8.8 Hz, 1H), 2.37 (t, J=10.8 Hz, 1H), 2.27 (s, 3H), 2.23 (J=6.8 Hz, 1H), 2.09-1.98 (m, 4H), 1.52 (t, J=12.8 Hz, 2H).

### Intermediate 24

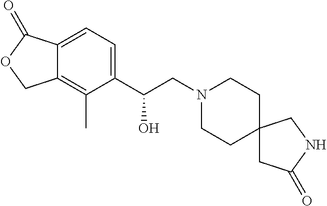 [SMILES: Cc1c([C@@H](O)CN2CCC3(CC2)CNC(=O)C3)ccc2c1COC2=O]

tert-Butyl 3-oxo-2, 8-diazaspiro[4.5]decane-8-carboxylate (INTERMEDIATE 15) (500 mg, 1.966 mmol) was dissolved in DCM (20 mL) and treated with 20 mL of 4N HCl in dioxane. After the reaction was stirred at room temperature for 4 hours, the excess of solvent was removed. The residue was then dissolved in EtOH, treated with DIEA (1717 μl, 9.83 mmol) and added to a sealed tube containing (R)-4-methyl-5-(oxiran-2-yl)isobenzofuran-1(3H)-one (374 mg, 1.966 mmol). The reaction vessel was sealed and heated at 100° C. overnight. The reaction was then cooled, concentrated and purified via MPLC eluting with 15% acetone/DCM to provide (R)-8-(2-hydroxy-2-(4-methyl-1-oxo-1,3-dihydroisobenzofuran-5-yl)ethyl)-2,8-diazaspiro[4.5]decan-3-one. LC-MS (IE, m/z): 345 [M+1]⁺.

### Intermediate 25

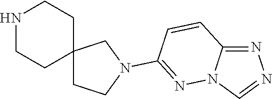 [SMILES: c1cc2nncn2nc1N1CCC2(CCNCC2)C1]

tert-Butyl 2,8-diazaspiro[4.5]decane-8-carboxylate (1.3 g, 5.42 mmol), 6-chloro-[1,2,4]triazolo[4,3-β]pyridazine (1.25 g, 8.11 mmol), diisopropylethylamine (2.74 ml, 15.6 mmol) and N,N-dimethylacetamide (8 ml) were mixed in a 40 mL vial. The mixture was heated at 80° C. for 16 hours. The solvent was then removed in Genavac to dryness to give the crude product, which was dissolved in 1,4-dioxanes (4 mL) and 4M HCl in 1,4-dioxanes (8 mL). The mixture was stirred for 3 hours at rt. The solvent was then removed to give a solid. Diethyl ether (10 mL) and ethyl acetate (10 mL) was added and the mixture was sonicated for 2 minutes. The solid was then filtered and dried under vacuum to give 6-(2,8-diazaspiro[4.5]decan-2-yl)-[1,2,4]triazolo[4,3-β]pyridazine as hydrochloric acid salt, which was used in the next step without further purification. LC-MS (IE, m/z): 259 [M+1]⁺.

### Intermediate 26

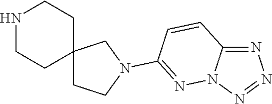 [SMILES: c1cc2nnnn2nc1N1CCC2(CCNCC2)C1]

### Step A: tert-butyl 2-(tetrazolo[1,5-b]pyridazin-6-yl)-2,8-diazaspiro[4.5]decane-8-carboxylate

tert-Butyl 2,8-diazaspiro[4.5]decane-8-carboxylate (0.150 g, 0.624 mmol), 6-chlorotetrazolo[1,5-β]pyridazine (0.097 g, 0.624 mmol), Huning's base (0.327 mL, 1.87 mmol) and 1,4-dioxane (2.0 mL) was charged to a microwave tube. The solution was degassed and filled with nitrogen (3×), then sealed and heated in a microwave reactor to 120° C. for 1 h. The reaction was cooled to room temperature and concentrated in vacuo. The resulting residue was purified by prep TLC (5% MeOH:DCM) to give the title compound. ¹HNMR (500 MHz, CDCl₃), δ 8.02 (m, 1H), 7.01 (m, 1H), 3.65 (s, 3H), 3.28 (m, 2H), 3.53 (m, 4H), 3.39 (m, 2H), 1.63 (m, 4H), 1.38 (s, 9H); LC-MS (IE, m/z): 360 [M+1]⁺.

### Step B: 6-(2,8-diazaspiro[4.5]decan-2-yl)tetrazolo[1,5-β]pyridazine

To a solution of tert-butyl 2-(tetrazolo[1,5-β]pyridazin-6-yl)-2,8-diazaspiro[4.5]decane-8-carboxylate (0.152 mg, 0.423 mmol) in dichloromethane (1.0 mL) at 0° C. was added trifluoroacetic acid (0.5 mL). The reaction was stirred at room temperature for 30 minutes. The mixture was concentrated in vacuo. The resulting residue was partitioned between DCM and saturated sodium bicarbonate solution which was adjusted with 1N NaOH to maintain pH˜9. The aqueous layer was extracted i-PrOH:CHCl₃ (1:3, 3×) and combined organic layers were washed with brine, dried (Mg₂SO₄), filtered and concentrated to provide the title compound. LC-MS (IE, m/z): 260 [M+1]+.

### Intermediate 27

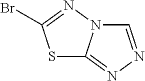

To a stirred solution of bromide (0.456 mL, 8.86 mmol) and isoamyl nitrite (1.91 mL, 14.2 mmol) in acetonitrile (5.0 ml) was added dropwise a solution of [1,2,4]triazolo[3,4-β][1,3,4]thiadiazol-6-amine (0.500 g, 3.54 mmol) in acetonitrile (5.0 mL). When the starting material was completely consumed (indicated by TLC and LC-MS), the mixture was quenched with saturated NaHCO₃ and EtOAc. The organic layer was washed with Na₂SO₃ and brine, then dried over Na₂SO₄, filtered and concentrated to provide 6-bromo-[1,2,4]triazolo[3,4-β][1,3,4]thiadiazole, which was used without further purification. ¹HNMR (500 MHz, CDCl₃), δ 8.97 (s, 1H); LC-MS (IE, m/z): 205, 207 [M+1]⁺.

### Intermediate 28

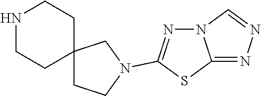 [SMILES: c1nnc2sc(N3CCC4(CCNCC4)C3)nn12]

6-(2,8-Diazaspiro[4.5]decan-2-yl)-[1,2,4]triazolo[3,4-β][1,3,4]thiadiazole was prepared in a similar fashion to that described for the synthesis of INTERMEDIATE 26 starting from tert-butyl 2,8-diazaspiro[4.5]decane-8-carboxylate and 6-bromo-[1,2,4]triazolo[3,4-β][1,3,4]thiadiazole (INTERMEDIATE 27). LC-MS (IE, m/z): 265 [M+1]⁺.

### Intermediate 29

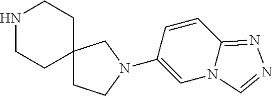 [SMILES: c1cc2nncn2cc1N1CCC2(CCNCC2)C1]

### Step A: tert-butyl 2-([1,2,4]triazolo[4,3-a]pyridin-6-yl)-2,8-diazaspiro[4.5]decane-8-carboxylate

tert-Butyl 2,8-diazaspiro[4.5]decane-8-carboxylate (0.200 g, 0.832 mmol), 6-bromo-[1,2,4]triazolo[4,3-a]pyridine (0.181 g, 0.915 mmol), cesium carbonate (0.407 mg, 1.25 mmol), Pd₂(dba)₃ (0.019 mg, 0.021 mmol), Xantphos (0.036 mg, 0.062 mmol), and 1,4-dioxane (3.0 mL) were charged to a microwave tube. The solution was degassed and filled with nitrogen (3×), then heated to 95° C. for 1 hour. The reaction was cooled to rt and concentrated in vacuo. The resulting residue was purified by prep TLC (5% MeOH/DCM) to provide tert-butyl 2-([1,2,4]triazolo[4,3-a]pyridin-6-yl)-2,8-diazaspiro[4.5]decane-8-carboxylate. LC-MS (IE, m/z): 358 [M+1]⁺.

### Step B: 6-(2,8-diazaspiro[4.5]decan-2-yl)-[1,2,4]triazolo[4,3-α]pyridine

The title compound was prepared from tert-butyl 2-([1,2,4]triazolo[4,3-a]pyridin-6-yl)-2,8-diazaspiro[4.5]decane-8-carboxylate in a similar fashion to that described for the synthesis of INTERMEDIATE 26 Step B. LC-MS (IE, m/z): 258 [M+1]⁺.

### Intermediate 30

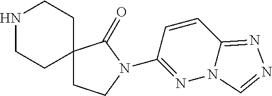 [SMILES: O=C1N(c2ccc3nncn3n2)CCC12CCNCC2]

2-([1,2,4]Triazolo[4,3-β]pyridazin-6-yl)-2,8-diazaspiro[4.5]decan-1-one was prepared in a similar fashion to that described for the synthesis of INTERMEDIATE 26 starting from tert-butyl 1-oxo-2,8-diazaspiro[4.5]decane-8-carboxylate (INTERMEDIATE 14) and 6-chloro-[1,2,4]triazolo[4,3-β]pyridazine. LC-MS (IE, m/z): 273 (M+1)⁺.

### Intermediate 31

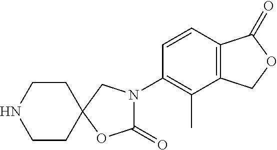 [SMILES: Cc1c(N2CC3(CCNCC3)OC2=O)ccc2c1COC2=O]

### Step A: tert-butyl 3-(4-methyl-1-oxo-1,3-dihydroisobenzofuran-5-yl)-2-oxo-1-oxa-3,8-diazaspiro[4.5]decane-8-carboxylate

tert-Butyl 2-oxo-1-oxa-3,8-diazaspiro[4.5]decane-8-carboxylate (INTERMEDIATE 16) (120 mg, 0.468 mmol), 4-methyl-1-oxo-1,3-dihydroisobenzofuran-5-yl trifluoromethanesulfonate (153 mg, 0.515 mmol), Pd₂(dba)₃ (10.72 mg, 0.012 mmol), Xantphos (20.32 mg, 0.035 mmol), and Cs₂CO₃ (229 mg, 0.702 mmol) were charged to a microwave vile. The vial was sealed, degased, and filled with dioxane (2.3 mL). The reaction mixture was heated at 95° C. overnight. The reaction was then diluted with water, extracted with EtOAc, and the organic layer was washed with brined, dried, and evaporated to give the crude product, which was purified by column chromatography (0-100% EtOAc/hexanes) to afford the title compound. LC/MS: [(M-56+1)]+=347.

### Step B: 3-(4-methyl-1-oxo-1,3-dihydroisobenzofuran-5-yl)-1-oxa-3,8-diazaspiro[4.5]decan-2-one

The title compound was prepared in a similar fashion to that described for the synthesis of INTERMEDIATE 26 Step B from tert-butyl 3-(4-methyl-1-oxo-1,3-dihydroisobenzofuran-5-yl)-2-oxo-1-oxa-3,8-diazaspiro[4.5]decane-8-carboxylate. LC/MS: [(M+1)]⁺=303.

### Intermediate 32

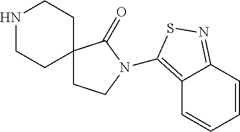 [SMILES: O=C1N(c2snc3ccccc23)CCC12CCNCC2]

2-(Benzo[γ]isothiazol-3-yl)-2,8-diazaspiro[4.5]decan-1-one was prepared in a similar fashion to that described for the synthesis of INTERMEDIATE 31 from tert-butyl 1-oxo-2,8-diazaspiro[4.5]decane-8-carboxylate (INTERMEDIATE 14) and 3-bromobenzo[γ]isothiazole. LC/MS: [(M+1)]⁺=288.

### Intermediate 33

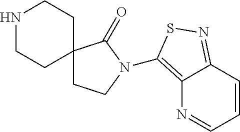 [SMILES: O=C1N(c2snc3cccnc23)CCC12CCNCC2]

2-(Isothiazolo[4,3-b]pyridin-3-yl)-2,8-diazaspiro[4.5]decan-1-one was prepared in a similar fashion to that described for the synthesis of INTERMEDIATE 31 from tert-butyl 1-oxo-2,8-diazaspiro[4.5]decane-8-carboxylate (INTERMEDIATE 14) and 3-bromoisothiazolo[4,3-β]pyridine. LC/MS: [(M+1)]⁺=289.

### Intermediate 34

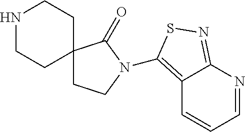 [SMILES: O=C1N(c2snc3ncccc23)CCC12CCNCC2]

2-(Isothiazolo[3,4-β]pyridin-3-yl)-2,8-diazaspiro[4.5]decan-1-one was prepared in a similar fashion to that described for the synthesis of INTERMEDIATE 31 from tert-butyl 1-oxo-2,8-diazaspiro[4.5]decane-8-carboxylate (INTERMEDIATE 14) and 3-bromoisothiazolo[3,4-β]pyridine. LC/MS: [(M+1)]⁺=289.

### Intermediate 35

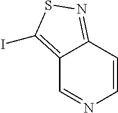

To a stirred solution of iodine (504 mg, 1.984 mmol) and isoamyl nitrite (427 μL, 3.17 mmol) in acetonitrile (6 mL) was added dropwise a acetonitrile (2 mL) solution of isothiazolo[4,3-γ]pyridin-3-amine (120 mg, 0.794 mmol). The reaction mixture was monitored by TLC and LCMS. After the starting material was consumed, the reaction mixture was absorbed on silica. Silica gel column chromatography (0-30% EtOAc/hexane) gave 3-iodoisothiazolo[4,3-γ]pyridine. ¹H NMR (500 MHz, CDCl₃) δ 8.90 (dd, J=3.8, 1.3 Hz, 1H), 8.16 (dd, J=8.9, 1.3 Hz, 1H), 7.42 (d, J=8.9, 3.8 Hz, 1H).

### Intermediate 36

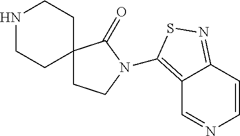 [SMILES: O=C1N(c2snc3ccncc23)CCC12CCNCC2]

2-(Isothiazolo[4,3-γ]pyridin-3-yl)-2,8-diazaspiro[4.5]decan-1-one was prepared in a similar fashion to that described for the synthesis of INTERMEDIATE 31 from tert-butyl 1-oxo-2,8-diazaspiro[4.5]decane-8-carboxylate (INTERMEDIATE 14) and 3-iodoisothiazolo[4,3-γ]pyridine (INTERMEDIATE 35). LC/MS: [(M+1)]⁺=289.

### Intermediate 37

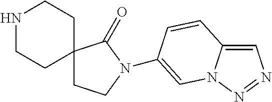 [SMILES: O=C1N(c2ccc3cnnn3c2)CCC12CCNCC2]

2-([1,2,3]triazolo[1,5-α]pyridin-6-yl)-2,8-diazaspiro[4.5]decan-1-one was prepared in a similar fashion to that described for the synthesis of INTERMEDIATE 31 from tert-butyl 1-oxo-2,8-diazaspiro[4.5]decane-8-carboxylate (INTERMEDIATE 14) and 6-bromo-[1,2,3]triazolo[1,5-α]pyridine. LC/MS: [(M+1)]⁺=272.

### Intermediate 38

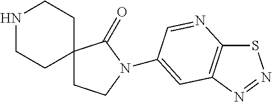 [SMILES: O=C1N(c2cnc3snnc3c2)CCC12CCNCC2]

2-([1,2,3]Thiadiazolo[5,4-b]pyridin-6-yl)-2,8-diazaspiro[4.5]decan-1-one was prepared in a similar fashion to that described for the synthesis of INTERMEDIATE 31 from tert-butyl 1-oxo-2,8-diazaspiro[4.5]decane-8-carboxylate (INTERMEDIATE 14) and 6-bromo-[1,2,3]thiadiazolo[5,4-β]pyridine. LC/MS: [(M+1)]⁺=290.

### Intermediate 39

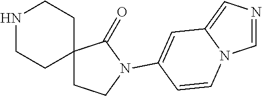 [SMILES: O=C1N(c2ccn3cncc3c2)CCC12CCNCC2]

2-(Imidazo[1,5-α]pyridin-7-yl)-2,8-diazaspiro[4.5]decan-1-one was prepared in a similar fashion to that described for the synthesis of INTERMEDIATE 31 from tert-butyl 1-oxo-2,8-diazaspiro[4.5]decane-8-carboxylate (INTERMEDIATE 14) and 7-bromoimidazo[1,5-a]pyridine. LC/MS: [(M+1)]⁺=271.

### Intermediate 40

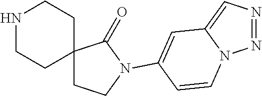 [SMILES: O=C1N(c2ccn3nncc3c2)CCC12CCNCC2]

2-([1,2,3]Triazolo[1,5-α]pyridin-5-yl)-2,8-diazaspiro[4.5]decan-1-one was prepared in a similar fashion to that described for the synthesis of INTERMEDIATE 31 from tert-butyl 1-oxo-2,8-diazaspiro[4.5]decane-8-carboxylate (INTERMEDIATE 14) and 5-bromo-[1,2,3]triazolo[1,5-α]pyridine. LC/MS: [(M+1)]⁺=272.

### Intermediates 41

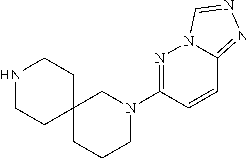 [SMILES: c1cc2nncn2nc1N1CCCC2(CCNCC2)C1]

6-(2,9-Diazaspiro[5.5]undecan-2-yl)-[1,2,4]triazolo[4,3-β]pyridazine was prepared in a similar fashion to that described for the synthesis of INTERMEDIATE 26 starting from tert-butyl 2,9-diazaspiro[5.5]undecane-9-carboxylate (INTERMEDIATE 13) and 6-chloro-[1,2,4]triazolo[4,3-β]pyridazine. LC/MS: [(M+1)]⁺=273.

### Intermediate 42

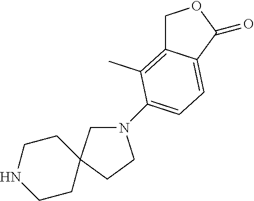 [SMILES: Cc1c(N2CCC3(CCNCC3)C2)ccc2c1COC2=O]

### Step A: tert-butyl 2-(4-methyl-1-oxo-1,3-dihydroisobenzofuran-5-yl)-2,8-diazaspiro[4.5]decane-8-carboxylate

A microwave vial containing tert-butyl 2,8-diazaspiro[4.5]decane-8-carboxylate (100 mg, 0.416 mmol), 5-bromo-4-methylisobenzofuran-1(3H)-one (94 mg, 0.416 mmol), Pd₂(dba)₃ (19.05 mg, 0.021 mmol), X-Phos (29.8 mg, 0.062 mmol) and potassium phosphate (177 mg, 0.832 mmol) in dioxane (2.080 mL) was sealed and evacuated and purged with nitrogen before heating to 100° C. for 1 h. The reaction was cooled, diluted with ethyl acetate, filtered and the filtrates concentrated to give crude material which was purified via MPLC (10-75% EtOAc/hexanes) to afford the title compound. LC/MS: [(M+1)]⁺=387.

### Step B: 4-methyl-5-(2,8-.5]decan-2-yl)isobenzofuran-1(3H)-one

tert-Butyl 2-(4-methyl-1-oxo-1,3-dihydroisobenzofuran-5-yl)-2,8-diazaspiro[4.5]decane-8-carboxylate was dissolved in DCM and then treated with 5 mL of 4N HCl at rt for 1 h. Excess solvent was then removed on the rotoevaporator. The residue was then re-dissolved in 4:1 chloroform/IPA and treated with 3 mL of 1N NaOH for 5 min and the then the solution was passed through a SPE column, washing with organic layer. The eluent was then concentrated to give the crude free amine which was used without further purification.

### Intermediate 43

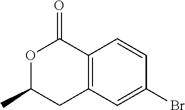

### (3R)-6-Bromo-3-methyl-3,4-dihydro-1H-isochromen-1-one

Absolute stereochemistry obtained from this enzymatic reaction was not identified or determined to be R or S, the stereochemistry was assigned arbitrarily as R.

### Step A: 4-bromo-N,N-diethyl-2-methylbenzamide

A solution of 4-bromo-2-methylbenzoic acid (25.0 g, 116 mmol) in DCM (400 mL) was treated with oxalyl chloride (11.7 mL, 134 mmol) and a catalytic amount of dry DMF (0.1 mL). The reaction was allowed to stir under nitrogen for 2 h at rt. Removal of excess solvent gave crude acid chloride which was redissolved in DCM (400 mL). The mixture was then cooled to 0° C. and triethyl amine (40.5 mL, 291 mmol) was added followed by the slow addition of diethyl amine (24.3 mL, 233 mmol). The reaction was then allowed to warm to rt overnight. The crude mixture was then diluted with 400 mL of water and extracted with DCM (3×500 mL). The combined organic layers were then washed with brine (200 mL), dried over magnesium sulfate, filtered and then concentrated. The crude material was purified via MPLC (10% EtOAc/Hex) to afford the title compound. ¹H NMR (500 MHz; CDCl₃) δ 7.39 (s, 1H), 7.36 (dd, J=1.6; 9.7 Hz, 1H), 7.05 (d, J=8.1, 1H), 3.3 (bs, 1H), 3.5 (bs, 1H), 3.13 (q, J=6.8 Hz, 2H), 2.29 (s, 3H), 1.27 (t, J=7.1 Hz, 3H), 1.05 (t, J=7.1 Hz, 3H). LC/MS: [(M+1)]⁺=270.

### Step B: 4-bromo-N,N-diethyl-2-(2-oxopropyl)benzamide

A 2M solution of LDA (35.2 mL, 70.3 mmol) in THF (176 mL) cooled to −78° C. was treated with slow addition of 4-bromo-N,N-diethyl-2-methylbenzamide (19 g, 70.3 mmol) in dry THF (176 mL). The reaction was allowed to stir at −78° C. for 1 h before it was quenched with N-methoxy-N-methylacetamide (22.43 mL, 211 mmol) and allowed to slowly warm to rt. The reaction was stirred overnight and then partitioned between 1N HCl (200 mL) and EtOAc (400 mL). The aqueous layer was further extracted with EtOAc (2×150 mL). The combined organic layers were washed with brine (150 mL), dried over magnesium sulfate, filtered and concentrated. The crude material was an orange/brown oil out of which the product crystalizes. The oil was decanted off and the solid was washed with hexanes and dried using a Buchner funnel to afford the title compound. ¹H NMR (500 MHz; CDCl₃) δ 7.44 (dd, J=1.7; 8.1 Hz, 1H), 7.37 (d, J=1.6 Hz, 1H), 7.11 (d, J=8.0 Hz, 1H), 3.81 (bs, 2H), 3.52 (bs, 2H), 3.18 (q, J=7.1 Hz, 2H), 2.21 (s, 3H), 1.21 (t, J=7.1 Hz, 3H), 1.10 (t, J=7.1 Hz, 3H). LC/MS: [(M+1)]⁺=312.

### Step C: 4-Bromo-N,N-diethyl-2-[(2R)-2-hydroxypropyl]benzamide

A flask equipped with an overhead stirrer was charged with pH=8 phosphate buffer (156 mL, 31.2 mmol) followed by D-glucose (1.298 g, 7.21 mmol) and then warmed to 30° C. Next, 135 mg glucose dehydrogenase and 270 mg NADP+disodium was added to the glucose/buffer solution at once, a homogeneous solution was obtained after 1 min of agitation. Next, 577 mg of enzymatic reductase P1 B2 was added to the reaction vessel and stirred at 500 rpm at 30° C. until the enzyme is wetted (about 40 min). Lastly, a solution of 4-bromo-N,N-diethyl-2-(2-oxopropyl)benzamide (1.5 g, 4.80 mmol) dissolved in DMSO (14.56 mL) (pre-warmed on stir plate to 30° C.) was added to the reaction over approximately 3 min and agitated at 30° C. (400 rpm) overnight. After 48 h the reaction was cooled to rt and then 75 g of potassium carbonate was added to the reaction in portions and stirred for 15 min until enzyme clumps together when stirring was stopped. Next, acetonitrile (50 mL) was poured into the reaction flask and the layers were thoroughly mixed. Stirring was stopped after 15-20 min, the layers allowed to separate and the upper layer decanted off. This was repeated two more times with additional 50 mL of acetonitrile. The combined organic layers were then filtered through a medium porosity funnel, concentrated and then 50 ml MTBE was added to the concentrate and stirred for 5 min and then transferred to a separatory funnel and the layers separated. The aqueous layer was extracted further another 50 mL of MTBE. The combined organic extracts were dried over magnesium sulfate, filtered and concentrated. Purification via MPLC (30-70% EtOAc/Hex) afforded 4-bromo-N,N-diethyl-2-(2-oxopropyl)benzamide. The absolute stereochemistry obtained from this enzymatic reaction was not identified or determined to be R or S; the stereochemistry was assigned arbitrarily as R.

### Step D: (3R)-6-Bromo-3-methyl-3,4-dihydro-1H-isochromen-1-one

A solution of 4-bromo-N,N-diethyl-2-[(2R)-2-hydroxypropyl]benzamide (12.2 g, 38.8 mmol) dissolved in 4N HCl in dioxane (200 mL) was stirred at room temperature and monitored by TLC. After 3 days the reaction was partitioned between EtOAc (300 mL) and water (300 mL). The aqueous phase was further extracted with EtOAc (2×250 mL). The combined organic layers were then washed with water (200 mL), brine (200 mL), dried over magnesium sulfate, filtered and concentrated. The crude material was then purified via MPLC (15-30% EtOAc/Hexane) to afford the title compound. ¹H NMR (500 MHz; CDCl₃) δ 7.98 (d, J=8.2 Hz, 1H), 7.56 (dd, J=1.5, 8.2 Hz, 1H), 7.45 (s, 1H), 4.71 (m, 1H), 2.94 (m, 2H), 1.55 (d, J=6.3 Hz, 3H). LC/MS: [(M+1)]⁺=241.

### Intermediate 44

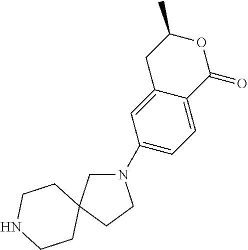 [SMILES: C[C@@H]1Cc2cc(N3CCC4(CCNCC4)C3)ccc2C(=O)O1]

### Step A: (R)-tert-butyl 2-(3-methyl-1-oxoisochroman-6-yl)-2,8-diazaspiro[4.5]decane-8-carboxylate

The title compound was prepared in an analogous fashion to that described for INTERMEDIATE 42, Step A from tert-butyl 2,8-diazaspiro[4.5]decane-8-carboxylate and (3R)-6-bromo-3-methyl-3,4-dihydro-1H-isochromen-1-one (INTERMEDIATE 43). LC/MS: [(M+1)]⁺=401.

### Step B: (R)-3-methyl-6-(2,8-diazaspiro[4.5]decan-2-yl)isochroman-1-one

The title compound (was prepared in an analogous fashion to that described for INTERMEDIATE 42, Step B from (R)-tert-butyl 2-(3-methyl-1-oxoisochroman-6-yl)-2,8-diazaspiro[4.5]decane-8-carboxylate.

### Intermediate 45

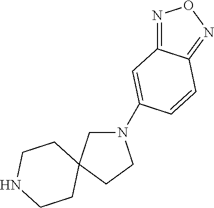 [SMILES: c1cc2nonc2cc1N1CCC2(CCNCC2)C1]

### Step A: tert-Butyl 2-(benzo[γ][1,2,5]oxadiazol-5-yl)-2,8-diazaspiro[4.5]decane-8-carboxylate

The title compound was prepared in an analogous fashion to that described for INTERMEDIATE 42, Step A. LC/MS: [(M+1)]⁺=359.

### Step B: 5-(2,8-Diazaspiro[4.5]decan-2-yl)benzo[γ ][1,2,5]oxadiazole

The title was prepared in an analogous fashion to that described for INTERMEDIATE 42, Step B from tert-butyl 2-(benzo[γ][1,2,5]oxadiazol-5-yl)-2,8-diazaspiro[4.5]decane-8-carboxylate.

### Intermediate 46

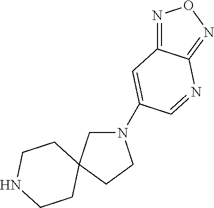 [SMILES: c1nc2nonc2cc1N1CCC2(CCNCC2)C1]

### Step A: tert-butyl 2-([1,2,5]oxadiazolo[3,4-β]pyridin-6-yl)-2,8-diazaspiro[4.5]decane-8-carboxylate

The title compound was prepared in an analogous fashion to that described for INTERMEDIATE 42, Step A. LC/MS: [(M+1)]⁺=360.

### Step B: 6-(2,8-diazaspiro[4.5]decan-2-yl)-[1,2,5]oxadiazolo[3,4-b]pyridine

The title compound was prepared in an analogous fashion to that described for INTERMEDIATE 42, Step B from tert-butyl 2-([1,2,5]oxadiazolo[3,4-(3]pyridin-6-yl)-2,8-diazaspiro[4.5]decane-8-carboxylate.

### Intermediate 47

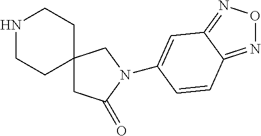 [SMILES: O=C1CC2(CCNCC2)CN1c1ccc2nonc2c1]

### Step A: tert-butyl 2-(benzo[γ][1,2,5]oxadiazol-5-yl)-3-oxo-2,8-diazaspiro[4.5]decane-8-carboxylate

A microwave vial containing tert-butyl 3-oxo-2,8-diazaspiro[4.5]decane-8-carboxylate (INTERMEDIATE 15) (200 mg, 0.786 mmol), 5-bromobenzo[γ][1,2,5]oxadiazole (156 mg, 0.786 mmol), Pd₂(dba)₃ (144 mg, 0.157 mmol), Xantphos (182 mg, 0.315 mmol) and cesium carbonate (384 mg, 1.18 mmol) in dioxane (3.9 mL) was sealed and evacuated and purged with nitrogen before heating in microwave to 120° C. for 12 min. The reaction was cooled, partitioned between EtOAc (150 mL) and water (40 mL). The organic phase was washed with brine, dried over magnesium sulfate, filtered and concentrated to give the crude material, which was purified via MPLC (30-80% EtOAc/hexanes) to afford tert-butyl 2-(benzo[γ][1,2,5]oxadiazol-5-yl)-3-oxo-2,8-diazaspiro[4.5]decane-8-carboxylate. LC-MS (372, m/z): 373 [M+1]+.

### Step B: 2-(benzo[γ][1,2,5]oxadiazol-5-yl)-2,8-diazaspiro[4.5]decan-3-one

The title compound was prepared in an analogous fashion to that described for INTERMEDIATE 42, Step B from tert-butyl 2-(benzo[γ][1,2,5]oxadiazol-5-yl)-3-oxo-2,8-diazaspiro[4.5]decane-8-carboxylate.

### Intermediate 48

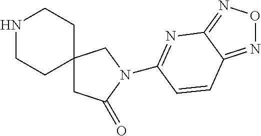 [SMILES: O=C1CC2(CCNCC2)CN1c1ccc2nonc2n1]

### Step A: tert-butyl 2-([1,2,5]oxadiazolo[3,4-β]pyridin-5-yl)-3-oxo-2,8-diazaspiro[4.5]decane-8-carboxylate

The title compound was prepared in an analogous fashion to that described for INTERMEDIATE 47. LC/MS: [(M+1)]⁺=374.

### Step B: 2-([1,2,5]Oxadiazolo[3,4-β]pyridin-5-yl)-2,8-diazaspiro[4.5]decan-3-one

The title compound was prepared in an analogous fashion to that described for INTERMEDIATE 42, Step B from the product of Step A.

### Intermediate 49

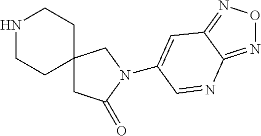 [SMILES: O=C1CC2(CCNCC2)CN1c1cnc2nonc2c1]

### Step A: tert-butyl 2-([1,2,5]oxadiazolo[3,4-β]pyridin-6-yl)-3-oxo-2,8-diazaspiro[4.5]decane-8-carboxylate

The title was prepared in an analogous fashion to that described for INTERMEDIATE 47, Step A. LC/MS: [(M+1)]⁺=374.

### Step B: 2-([1,2,5]xadiazolo[3,4-β]pyridin-6-yl)-2,8-diazaspiro[4.5]decan-3-one

2-([1,2,5]Oxadiazolo[3,4-β]pyridin-6-yl)-2,8-diazaspiro[4.5]decan-3-one was prepared in an analogous fashion to that described for INTERMEDIATE 42, Step B from tert-butyl 2-([1,2,5]oxadiazolo[3,4-β]pyridin-6-yl)-3-oxo-2,8-diazaspiro[4.5]decane-8-carboxylate.

### Intermediate 50

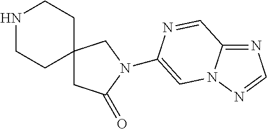 [SMILES: O=C1CC2(CCNCC2)CN1c1cn2ncnc2cn1]

### 2-([1,2,4]Triazolo[1,5-α]pyrazin-6-yl)-2,8-diazaspiro[4.5]decan-3-one

tert-Butyl 3-oxo-2,8-diazaspiro[4.5]decane-8-carboxylate (INTERMEDIATE 15) (50 mg, 0.197 mmol), 6-bromo[1,2,4]triazolo[1,5-α]pyrazine (58.8 mg, 0.296 mmol), copper(I) iodide (37.4 mg, 0.194 mmol), N,N′-dimethylethyldiamine (34.7 mg, 0.393 mmol) and cesium carbonate (192 mg, 0.59 mmol) were mixed in a 8 mL vial. 1,4-dioxanes (1 ml) was added. The vial was then capped and the mixture was heated at 98° C. overnight. The reaction mixture was cooled to room temperature, water (1 mL) and ethyl acetate (3 mL) were added. The organic layer was then collected. Removal of solvent gave the crude product, to which was added HCl in 1,4-dioxane (4M, 1 mL). The mixture was stirred at room temperature overnight. The solvent was then removed in vacuo to give the crude 2-([1,2,4]triazolo[1,5-α]pyrazin-6-yl)-2,8-diazaspiro[4.5]decan-3-one which was used without further purification.

### Intermediate 51

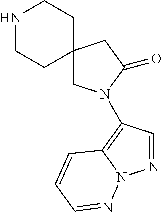 [SMILES: O=C1CC2(CCNCC2)CN1c1cnn2ncccc12]

2-(Pyrazolo[1,5-β]pyridazin-3-yl)-2,8-diazaspiro[4.5]decan-3-one was prepared in analogous fashion to that described for INTERMEDIATE 50 from tert-butyl 3-oxo-2,8-diazaspiro[4.5]decane-8-carboxylate (INTERMEDIATE 15) and 3-bromopyrazolo[1,5-β]pyridazine.

### Intermediate 52

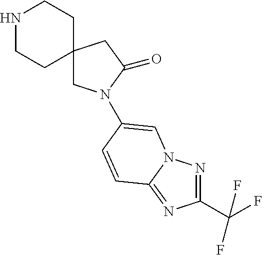 [SMILES: O=C1CC2(CCNCC2)CN1c1ccc2nc(C(F)(F)F)nn2c1]

2-(2-(Trifluoromethyl)-[1,2,4]triazolo[1,5-α]pyridin-6-yl)-2,8-diazaspiro[4.5]decan-3-one was prepared in analogous fashion to that described for INTERMEDIATE 50 from tert-butyl 3-oxo-2,8-diazaspiro[4.5]decane-8-carboxylate (INTERMEDIATE 15) and 6-bromo-2-(trifluoromethyl)-[1,2,4]triazolo[1,5-α]pyridine. LC-MS: 340 [M+1]⁺.

### Intermediate 53

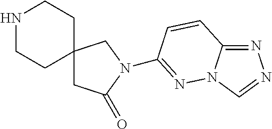 [SMILES: O=C1CC2(CCNCC2)CN1c1ccc2nncn2n1]

### Step A: tert-butyl 2-([1,2,4]triazolo[4,3-β]pyridazin-6-yl)-3-oxo-2,8-diazaspiro[4.5]decane-8-carboxylate

A mixture of tert-butyl 3-oxo-2,8-diazaspiro[4.5]decane-8-carboxylate (INTERMEDIATE 15) (417 mg, 1.64 mmol), 6-chloro-[1,2,4]triazolo[4,3-β]pyridazine (253 mg, 1.64 mmol), and potassium carbonate (453 mg, 3.28 mmol) in DMA (3 mL) was heated at 100° C. overnight. LC-MS analysis indicated the completion of the reaction. The mixture was diluted with EtOAc (30 mL) and water (15 mL). The organic layer was collected, dried and concentrated to give the title compound. LC/MS: [(M+1)]⁺=374.

### Step B: 2-([1,2,4]triazolo[4,3-β]pyridazin-6-yl)-2,8-diazaspiro[4.5]decan-3-one

The compound from Step A was dissolved in 3 mL of 4N HCl in 1,4-dioxane. The mixture was stirred at rt overnight. LC-MS showed the completion of the reaction. Removal of solvent gave a solid which was filtered and washed with EtOAc. The crude 2-([1,2,4]triazolo[4,3-β]pyridazin-6-yl)-2,8-diazaspiro[4.5]decan-3-one thus obtained was without further purification.

### Intermediate 54

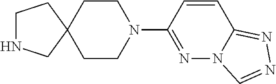 [SMILES: c1cc2nncn2nc1N1CCC2(CCNC2)CC1]

### Step A: tert-butyl 8-([1,2,4]triazolo[4,3-β]pyridazin-6-yl)-2,8-diazaspiro[4.5]decane-2-carboxylate

tert-Butyl 2,8-diazaspiro[4.5]decane-2-carboxylate (1.0 g, 4.16 mmol), 6-chloro-[1,2,4]triazolo[4,3-β]pyridazine (0.707 g, 4.58 mmol), and DIEA (1.45 mL, 8.32 mmol) were mixed in DMA (5 mL). The mixture was heated at 95° C. overnight. The solvent was removed to give the crude tert-butyl 8-([1,2,4]triazolo[4,3-β]pyridazin-6-yl)-2,8-diazaspiro[4.5]decane-2-carboxylate. LC/MS: [(M+1)]⁺=359.

### Step B: 6-(2,8-diazaspiro[4.5]decan-8-yl)-[1,2,4]triazolo[4,3-β]pyridazine

The crude tert-butyl 8-([1,2,4]triazolo[4,3-β]pyridazin-6-yl)-2,8-diazaspiro[4.5]decane-2-carboxylate from Step A was treated with 5 mL of 4N HCl in dioxane. The mixture was stirred at room temperature overnight then heated at 60° C. for 3 hours until complete removal of Boc group. The solvent was removed and the resulting solid was triturated with EtOAc to give 6-(2,8-diazaspiro[4.5]decan-8-yl)-[1,2,4]triazolo[4,3-β]pyridazine, which was used without further purification.

### Intermediate 55

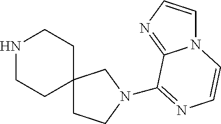 [SMILES: c1cn2ccnc2c(N2CCC3(CCNCC3)C2)n1]

### Step A: tert-Butyl 2-(imidazo[1,2-α]pyrazin-8-yl)-2,8-diazaspiro[4.5]decane-8-carboxylate

A vial containing tert-butyl 2,8-diazaspiro[4.5]decane-8-carboxylate (263 mg, 1.09 mmol), 8-chloroimidazo[1,2-α]pyrazine (168 mg, 1.09 mmol) and triethyl amine (152 uL, 1.09 mmol) in THF (5.4 mL) heated to 50° C. for 4 hours. The reaction was cooled and concentrated to give the crude material, which was purified via MPLC (50-100% EtOAc/hexanes) to afford the title compound. LC-MS (357, m/z): 358 [M+1]⁺.

### Step B: 8-(2,8-Diazaspiro[4.5]decan-2-yl)imidazo[1,2-α]pyrazine

The title compound was prepared in analogous fashion to that described for INTERMEDIATE 42, Step B from tert-butyl 2-(imidazo[1,2-α]pyrazin-8-yl)-2,8-diazaspiro[4.5]decane-8-carboxylate.

### Intermediate 56

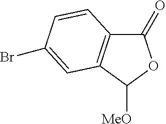 [SMILES: COC1OC(=O)c2ccc(Br)cc21]

5-Bromoisobenzofuran-1(3H)-one (1.0 g, 4.69 mmol), NBS (835 mg, 4.69 mmol), and carbon tetrachloride (15.6 mL) were heated to reflux in a 50 mL flask carrying a reflux condenser equipped with a drying tube. The reaction mixture was exposed to light of an ordinary 100-W unfrosted light bulb placed 6-8″ from the flask. After 30 min, the succinimide was removed by filtration and the filtrate was concentrated under atmospheric pressure to give crude 3,5-dibromoisobenzofuran-1(3H)-one. To 3,5-dibromoisobenzofuran-1(3H)-one was added methanol directly to afford 5-bromo-3-methoxyisobenzofuran-1(3H)-one. LC/MS: [(M+1)]⁺=244

### Intermediate 57A and 57B

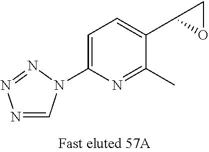

### (R)-2-Methyl-3-(oxiran-2-yl)-6-(1H-tetrazol-1-yl)pyridine

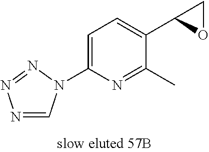

### (S)-2-Methyl-3-(oxiran-2-yl)-6-(1H-tetrazol-1-yl)pyridine

The above compounds were prepared in analogous fashion to that described for (R)-5-(oxiran-2-yl)-2-(1H-tetrazol-1-yl)pyridine and (S)-5-(oxiran-2-yl)-2-(1H-tetrazol-1-yl)pyridine (INTERMEDIATE 12A and 12B).

### Intermediate 58

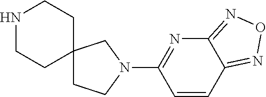 [SMILES: c1cc2nonc2nc1N1CCC2(CCNCC2)C1]

5-(2,8-Diazaspiro[4.5]decan-2-yl)-[1,2,5]oxadiazolo[3,4-b]pyridine was prepared in a similar fashion to that described for 6-(2,8-diazaspiro[4.5]decan-2-yl)-[1,2,4]triazolo[4,3-β]pyridazine (INTERMEDIATE 25).

### Example 1

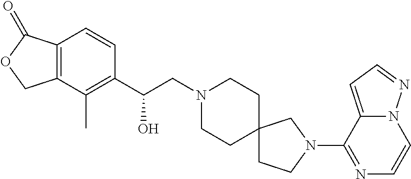 [SMILES: Cc1c([C@@H](O)CN2CCC3(CC2)CCN(c2nccn4nccc24)C3)ccc2c1COC2=O]

5-[(1R)-2-(2,8-diazaspiro[4.5]dec-8-yl)-1-hydroxyethyl]-4-methyl-2-benzofuran-1(3H)-one dihydrochloride (INTERMEDIATE 17) (30 mg, 0.074 mmole), 4-bromopyrazolo[1,5-a]pyrazine (22 mg, 0.112 mmole) and diisopropyl ethylamine (0.048 ml, 0.272 mmole) were mixed in 0.5 ml of N,N-dimethylacetamide in a vial. The mixture was stirred at 60° C. overnight. Analysis of the crude mixture by LCMS indicated the completion of the reaction. The mixture was cooled down and diluted with 0.5 mL of DMSO. The mixture was then purified by the reverse phase mass directed preparative HPLC system using CH₃CN/water as the mobile phase to give (R)-5-(1-hydroxy-2-(2-(pyrazolo[1,5-α]pyrazin-4-yl)-2,8-diazaspiro[4.5]decan-8-yl)ethyl)-4-methylisobenzofuran-1(3H)-one. ¹H NMR (600 MHz flow NMR, d6-DMSO) δ ppm 7.87 (1H, J=2.4 Hz), 7.85 (1H, J=4.8 Hz), 7.65 (2H, m), 7.22 (1H, J=4.8 Hz), 6.98 (1H, m), 5.35 (2H, dd, J=18.6, 15.6 Hz), 5.06 (1H, m), 3.8 (2H, br), 3.55 (2H, m), 3.37 (4H, m), 2.43 (2H, m), 2.23 (3H, s), 1.82 (2H, t, J=7.2 Hz), 1.55 (4H, m).

LC-MS (IE, m/z): 448.34 (M+1)+.

The compounds in Table 1 were prepared in an analogous fashion to Example 1 starting from INTERMEDIATE 17, 18, 20, 21, or 22 and the corresponding halide. The column in Table 1 with the heading INT provides the numbers which represent the intermediates that were used in the syntheses.

TABLE 1

| EX. | INT. | EXAMPLE STRUCTURE/NAME | LC/MS (M + 1)⁺ |
|---|---|---|---|
| 2 | 17 | 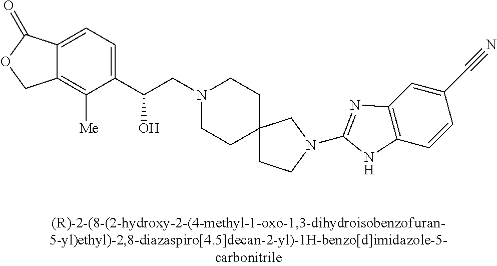 [SMILES: Cc1c([C@@H](O)CN2CCC3(CC2)CCN(c2nc4cc(C#N)ccc4[nH]2)C3)ccc2c1COC2=O] | 472 |
| 3 | 17 | 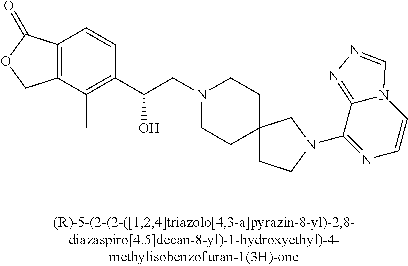 [SMILES: Cc1c([C@@H](O)CN2CCC3(CC2)CCN(c2nccn4cnnc24)C3)ccc2c1COC2=O] | 449 |
| 4 | 17 | 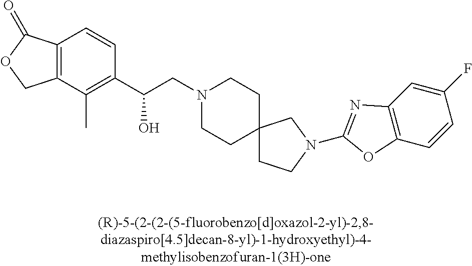 [SMILES: Cc1c([C@@H](O)CN2CCC3(CC2)CCN(c2nc4cc(F)ccc4o2)C3)ccc2c1COC2=O] | 466 |
| 5 | 17 | 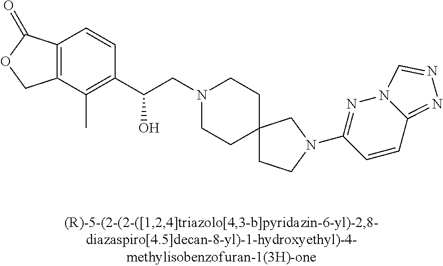 [SMILES: Cc1c([C@@H](O)CN2CCC3(CC2)CCN(c2ccc4nncn4n2)C3)ccc2c1COC2=O] | 449 |
| 6 | 17 | 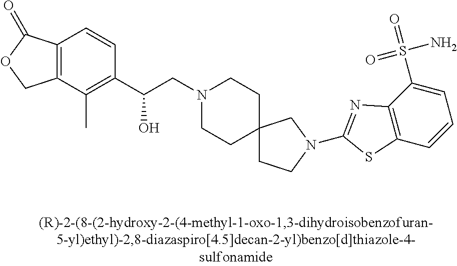 [SMILES: Cc1c([C@@H](O)CN2CCC3(CC2)CCN(c2nc4c(S(N)(=O)=O)cccc4s2)C3)ccc2c1COC2=O] | 543 |
| 7 | 17 | 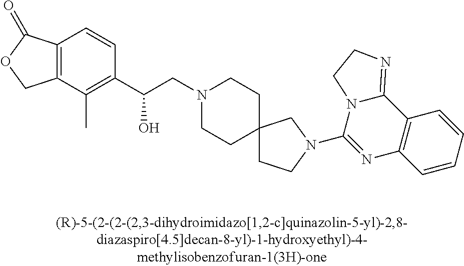 [SMILES: Cc1c([C@@H](O)CN2CCC3(CC2)CCN(C2=Nc4ccccc4C4=NCCN42)C3)ccc2c1COC2=O] | 500 |
| 8 | 17 | 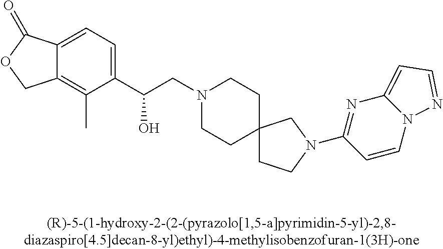 [SMILES: Cc1c([C@@H](O)CN2CCC3(CC2)CCN(c2ccn4nccc4n2)C3)ccc2c1COC2=O] | 448 |
| 9 | 17 | 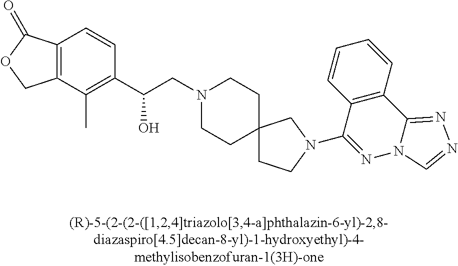 [SMILES: Cc1c([C@@H](O)CN2CCC3(CC2)CCN(c2nn4cnnc4c4ccccc24)C3)ccc2c1COC2=O] | 499 |
| 10 | 17 | 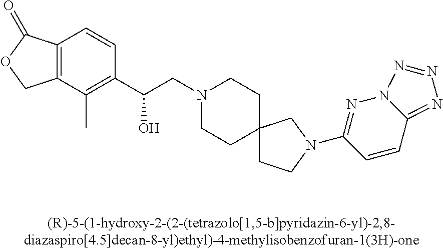 [SMILES: Cc1c([C@@H](O)CN2CCC3(CC2)CCN(c2ccc4nnnn4n2)C3)ccc2c1COC2=O] | 450 |
| 11 | 17 | 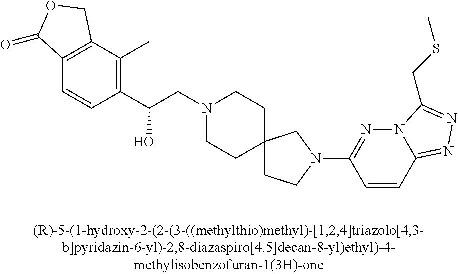 [SMILES: CSCc1nnc2ccc(N3CCC4(CCN(C[C@H](O)c5ccc6c(c5C)COC6=O)CC4)C3)nn12] | 509 |
| 12 | 17 | 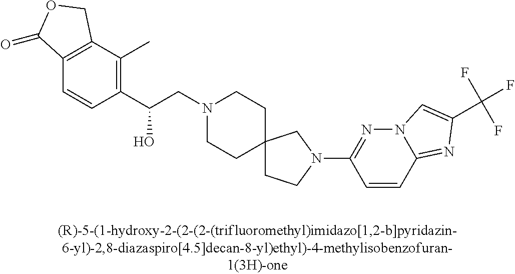 [SMILES: Cc1c([C@@H](O)CN2CCC3(CC2)CCN(c2ccc4nc(C(F)(F)F)cn4n2)C3)ccc2c1COC2=O] | 516 |
| 13 | 17 | 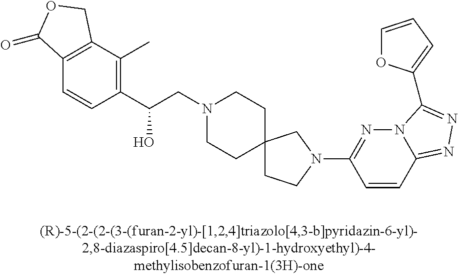 [SMILES: Cc1c([C@@H](O)CN2CCC3(CC2)CCN(c2ccc4nnc(-c5ccco5)n4n2)C3)ccc2c1COC2=O] | 515 |
| 14 | 17 | 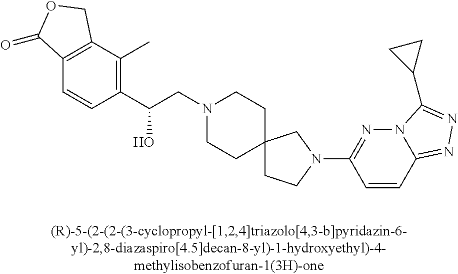 [SMILES: Cc1c([C@@H](O)CN2CCC3(CC2)CCN(c2ccc4nnc(C5CC5)n4n2)C3)ccc2c1COC2=O] | 489 |
| 15 | 17 | 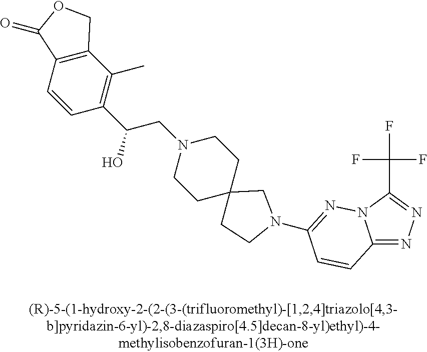 [SMILES: Cc1c([C@@H](O)CN2CCC3(CC2)CCN(c2ccc4nnc(C(F)(F)F)n4n2)C3)ccc2c1COC2=O] | 517 |
| 16 | 17 | 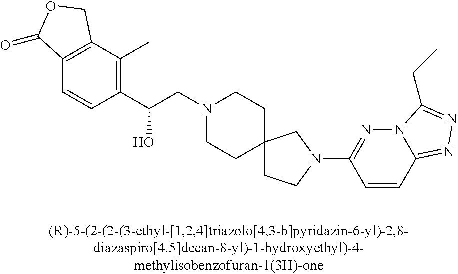 [SMILES: CCc1nnc2ccc(N3CCC4(CCN(C[C@H](O)c5ccc6c(c5C)COC6=O)CC4)C3)nn12] | 477 |
| 17 | 17 | 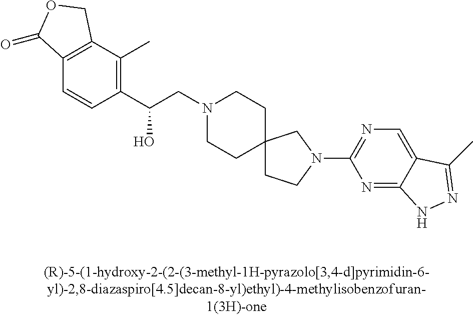 [SMILES: Cc1c([C@@H](O)CN2CCC3(CC2)CCN(c2ncc4c(C)n[nH]c4n2)C3)ccc2c1COC2=O] | 463 |
| 18 | 17 | 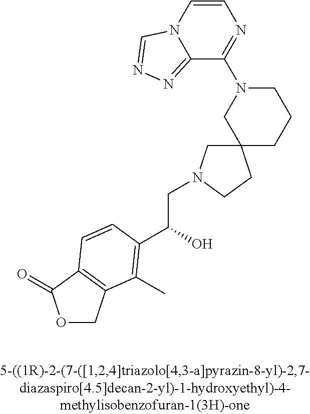 [SMILES: Cc1c([C@@H](O)CN2CCC3(CCCN(c4nccn5cnnc45)C3)C2)ccc2c1COC2=O] | 449 |
| 19 | 17 | 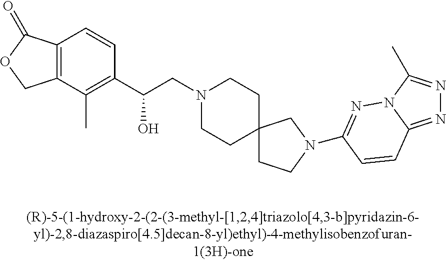 [SMILES: Cc1c([C@@H](O)CN2CCC3(CC2)CCN(c2ccc4nnc(C)n4n2)C3)ccc2c1COC2=O] | 463 |
| 20 | 21 | 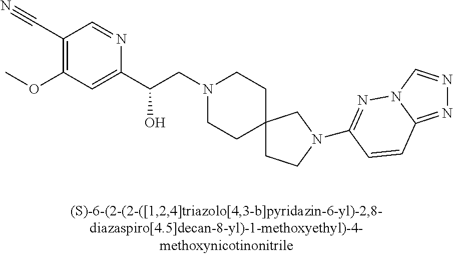 [SMILES: COc1cc([C@@H](O)CN2CCC3(CC2)CCN(c2ccc4nncn4n2)C3)ncc1C#N] | 431 |
| 21 | 20 | 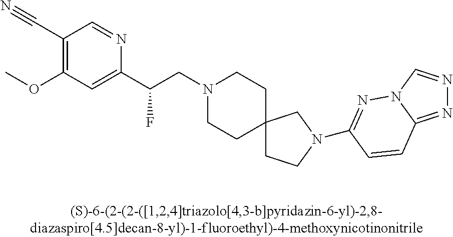 [SMILES: COc1cc([C@@H](F)CN2CCC3(CC2)CCN(c2ccc4nncn4n2)C3)ncc1C#N] | 437 |
| 22 | 22 | 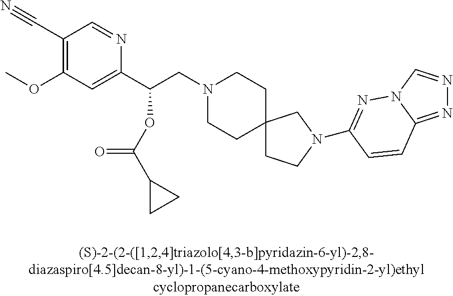 [SMILES: COc1cc([C@H](CN2CCC3(CC2)CCN(c2ccc4nncn4n2)C3)OC(=O)C2CC2)ncc1C#N] | 503 |

### Example 23

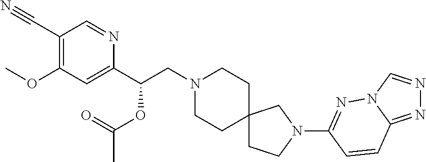 [SMILES: COc1cc([C@H](CN2CCC3(CC2)CCN(c2ccc4nncn4n2)C3)OC(C)=O)ncc1C#N]

To a solution of 6-(2-(2-([1,2,4]triazolo[4,3-b]pyridazin-6-yl)-2,8-diazaspiro[4.5]decan-8-yl)-1-hydroxyethyl)-4-methoxynicotinonitrile (EXAMPLE 45) (0.040 g, 0.092 mmol) in pyridine (1.0 mL) at room temperature was added acetic anhydride (0.026 mL, 0.276 mmol) and the mixture was stirred for 8 hours. The mixture was poured into water and extracted with EtOAc (3×). The combined organic layers were washed with water (2×), brine, then dried (Na₂SO₄), filtered and concentrated. The resulting residue was purified by prep TLC (5% MeOH:DCM) to give (S)-2-(2-([1,2,4]triazolo[4,3-β]pyridazin-6-yl)-2,8-diazaspiro[4.5]decan-8-yl)-1-(5-cyano-4-methoxypyridin-2-yl)ethyl acetate. LC-MS (IE, m/z): 477 (M+1)⁺.

### Example 24

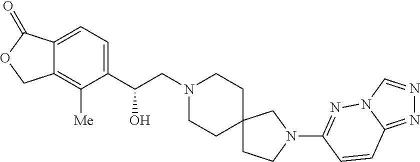 [SMILES: Cc1c([C@@H](O)CN2CCC3(CC2)CCN(c2ccc4nncn4n2)C3)ccc2c1COC2=O]

5-[(1R)-2-(2,8-diazaspiro[4.5]dec-8-yl)-1-hydroxyethyl]-4-methyl-2-benzofuran-1(3H)-one (INTERMEDIATE 17) (30 mg, 0.091 mmol), 6-bromo[1,2,4]triazolo[4,3-a]pyrimidine (27.1 mg, 0.136 mmol), copper(I) iodide (3.5 mg, 0.018 mmole), s-proline (10.5 mg, 0.091 mmol) and cesium carbonate (118 mg, 0.363 mmol) were mixed in a 8 ml vial. DMF (1 ml) was added. The vial was then capped and the mixture was heated at 95° C. overnight. The reaction mixture was cooled to rt, water (1 mL) and ethyl acetate (3 mL) were added. The organic layer was then collected. Removal of solvent gave crude product, which was then dissolved in 1 mL of DMSO and purified by reverse phase mass directed HPLC system using acetonitrile/water/formic acid as the mobile phase to give (R)-5-(2-(2-([1,2,4]triazolo[4,3-a]pyrimidin-6-yl)-2,8-diazaspiro[4.5]decan-8-yl)-1-hydroxyethyl)-4-methylisobenzofuran-1(3H)-one. LC-MS (IE, m/z): 449.46 (M+1)⁺.

### Example 25

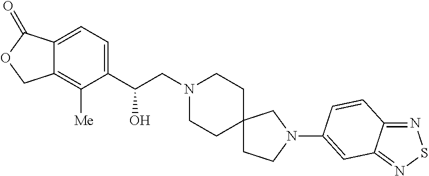 [SMILES: Cc1c([C@@H](O)CN2CCC3(CC2)CCN(c2ccc4nsnc4c2)C3)ccc2c1COC2=O]

In a 1 dram vial containing 1 mL of degassed (N₂) N,N′-dimethylamide, were added (R)-5-(1-hydroxy-2-(2,8-diazaspiro[4.5]decan-8-yl)ethyl)-4-methylisobenzofuran-1(3H)-one dihydrochloride (INTERMEDIATE 17) (52.4 mg, 0.13 mmol), (9,9-dimethyl-9H-xanthene-4,5-diyl)bis(diphenylphosphine) (12.01 mg, 0.021 mmol), tris(dibenzylideneacetone)dipalladium (0) (4.75 mg, 5.2 umol), 5-bromobenzo[c][1,2,5]thiadiazole (34 mg, 0.156 mmol) and cesium carbonate (101 mg, 0.311 mmol). The vial was purged with N₂ gas, sealed and heated at 85° C. overnight. The reaction mixture was cooled to room temperature, filtered and the filtrates were concentrated. The residue was purified by semi-preparative HPLC (focused gradient 0-40% ACN over 12 minutes using 0.1% TFA as the acidic modifier). The pure fractions were combined and the solvents were removed in vacuo. The final product was dissolved in 1 mL of 4:1 mixture of water/acetonitrile and lyophilized to dryness to give (R)-5-(2-(2-(benzo[c][1,2,5]thiadiazol-5-yl)-2,8-diazaspiro[4.5]decan-8-yl)-1-hydroxyethyl)-4-methylisobenzofuran-1 (3H)-one. LC-MS (IE, m/z): 465 (M+1)⁺.

### Example 26

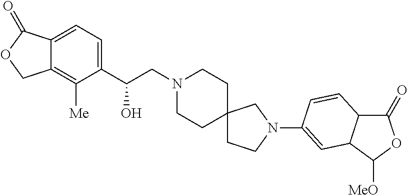 [SMILES: COC1OC(=O)C2C=CC(N3CCC4(CCN(C[C@H](O)c5ccc6c(c5C)COC6=O)CC4)C3)=CC12]

To a microwave vial was charged (R)-5-(1-hydroxy-2-(2,8-diazaspiro[4.5]decan-8-yl)ethyl)-4-methylisobenzofuran-1(3H)-one (INTERMEDIATE 17) (60 mg, 0.182 mmol), 5-bromo-3-methoxyisobenzofuran-1(3H)-one (INTERMEDIATE 56) (48.5 mg, 0.200 mmol), Pd2(dba)3 (8.31 mg, 9.08 μmol), X-Phos (17.31 mg, 0.036 mmol), and K₃PO₄ (77 mg, 0.363 mmol). The vial was sealed, degased, and filled with dioxane (908 μL). The reaction mixture was heated at 100° C. overnight, and diluted with water, extracted with EtOAc. The organic layer was washed with brined, dried, evaporated to give the crude product, which was purified by column chromatography (0-100% EtOAc/hex) to give 5-((1R)-1-hydroxy-2-(2-(3-methoxy-1-oxo-1,3-dihydroisobenzofuran-5-yl)-2,8-diazaspiro[4.5]decan-8-yl)ethyl)-4-methylisobenzofuran-1(3H)-one. LC/MS: [(M+1)]⁺=493

### Example 27

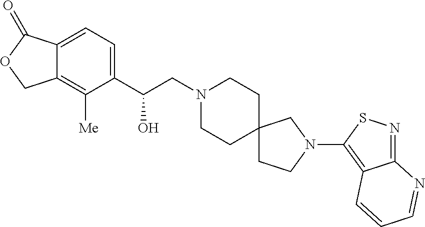 [SMILES: Cc1c([C@@H](O)CN2CCC3(CC2)CCN(c2snc4ncccc24)C3)ccc2c1COC2=O]

Using a glove box, 3-bromoisothiazolo[3,4-b]pyridine (32 mg, 0.15 mmol) and cesium carbonate (0.158 g, 0.484 mmol) were dispensed into a 4 mL vial. (R)-5-(1-hydroxy-2-(2,8-diazaspiro[4.5]decan-8-yl)ethyl)-4-methylisobenzofuran-1(3H)-one (INTERMEDIATE 17) (0.040 g, 0.121 mmol) was dissolved in t-amyl alcohol (1 mL), which had been degassed for 1 h and this solution was added to the vial. 2-Dicyclohexylphosphino-2′,6′-diisopropoxybiphenyl (RuPhos) (2.82 mg, 6.05 μmol) and 1^{st} generation RuPhos pre-catalyst (6.05 μmol) were dissolved in t-amyl alcohol (1 mL) and this solution was added to the vial containing the prior reactants. The vial was capped and heated at 95° C. overnight. Post reaction workup entailed solvent removal (Gene Vac), dissolution in 1.5 mL of DMSO, filtration and semi-preparative HPLC purification afforded the (R)-5-(1-hydroxy-2-(2-(isothiazolo[3,4-b]pyridin-3-yl)-2,8-diazaspiro[4.5]decan-8-yl)ethyl)-4-methylisobenzofuran-1(3H)-one. LC-MS (IE, m/z): 465 (M+1)⁺.

### Example 28

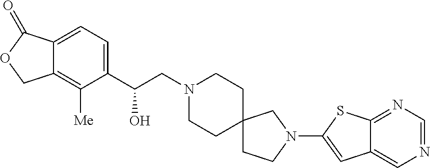 [SMILES: Cc1c([C@@H](O)CN2CCC3(CC2)CCN(c2cc4cncnc4s2)C3)ccc2c1COC2=O]

(R)-5-(1-Hydroxy-2-(2-(thieno[2,3-d]pyrimidin-6-yl)-2,8-diazaspiro[4.5]decan-8-yl)ethyl)-4-methylisobenzofuran-1 (3H)-one was prepared in analogous fashion to that described for (R)-5-(1-hydroxy-2-(2-(isothiazolo[3,4-b]pyridin-3-yl)-2,8-diazaspiro[4.5]decan-8-yl)ethyl)-4-methylisobenzofuran-1(3H)-one (example 29). LC-MS (IE, m/z): 465 (M+1)⁺.

### Example 29

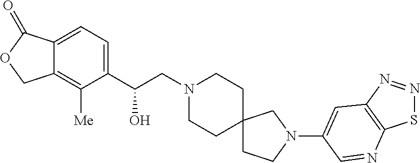 [SMILES: Cc1c([C@@H](O)CN2CCC3(CC2)CCN(c2cnc4snnc4c2)C3)ccc2c1COC2=O]

To a one dram vial were dispensed 6-bromo-[1,2,3]thiadiazolo[5,4-b]pyridine (28 mg, 0.13 mmol) and cesium carbonate (0.097 g, 0.298 mmol). Next, (R)-5-(1-hydroxy-2-(2,8-diazaspiro[4.5]decan-8-yl)ethyl)-4-methylisobenzofuran-1(3H)-one dihydrochloride (INTERMEDIATE 17) (0.040 g, 0.099 mmol), which was dissolved in 0.5 mL of 1,4-dioxane that had been degassed under nitrogen for 1 hour, was added to the vial prepared. Next, a mixture containing 4,5-bis(diphenylphosphino)-9,9-dimethylxanthene (Xantphos) (11 mg, 0.020 mmol) and tris (dibenzylideneacetone)dipalladium (0) (4.54 mg, 4.96 μmol) 1 mL of degassed dioxane was prepared and the mixture was then added to the vial containing the halide and the core. A stir bar was added to the vial and the vial was blanketed with nitrogen and capped. The vial was heated at 95° C. overnight. The reaction mixture was cooled to room temperature and filtered. The filtrates were concentrated and the resulting oil was purified by RP-HPLC and combined pure fraction yielded (R)-5-(2-(2-([1,2,3]thiadiazolo[5,4-b]pyridin-6-yl)-2,8-diazaspiro[4.5]decan-8-yl)-1-hydroxyethyl)-4-methylisobenzofuran-1(3H)-one. LC-MS (IE, m/z): 466 (M+1)⁺.

The compounds in Table 2 were prepared in an analogous fashion to EXAMPLE 29 from INTERMEDIATE 23 and the corresponding halide.

TABLE 2

| | | | LC/MS, |
|---|---|---|---|
| EX. | INT. | EXAMPLE STRUCTURE/NAME | (M + 1)⁺ |
| 30 | 23 | 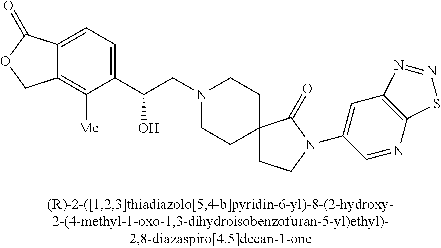 [SMILES: Cc1c([C@@H](O)CN2CCC3(CC2)CCN(c2cnc4snnc4c2)C3=O)ccc2c1COC2=O] | 480 |
| 31 | 23 | 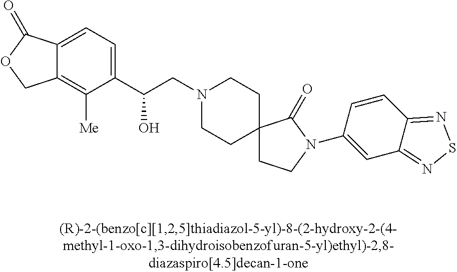 [SMILES: Cc1c([C@@H](O)CN2CCC3(CC2)CCN(c2ccc4nsnc4c2)C3=O)ccc2c1COC2=O] | 479 |
| 32 | 23 | 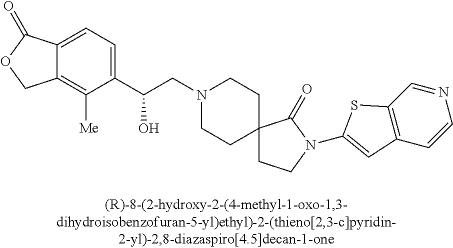 [SMILES: Cc1c([C@@H](O)CN2CCC3(CC2)CCN(c2cc4ccncc4s2)C3=O)ccc2c1COC2=O] | 478 |
| 33 | 23 | 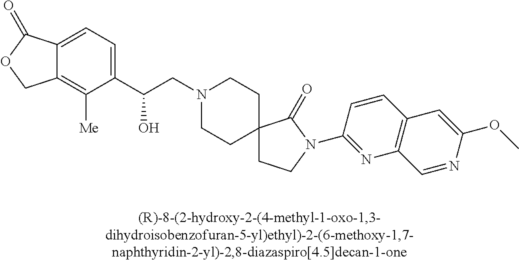 [SMILES: COc1cc2ccc(N3CCC4(CCN(C[C@H](O)c5ccc6c(c5C)COC6=O)CC4)C3=O)nc2cn1] | 503 |
| 34 | 23 | 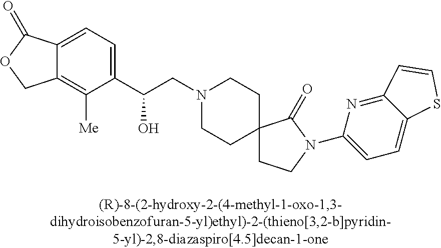 [SMILES: Cc1c([C@@H](O)CN2CCC3(CC2)CCN(c2ccc4sccc4n2)C3=O)ccc2c1COC2=O] | 478 |
| 35 | 23 | 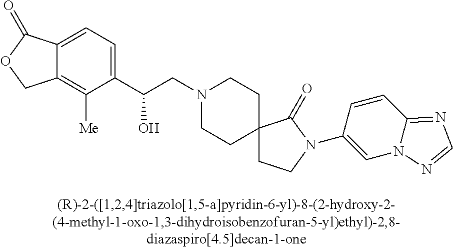 [SMILES: Cc1c([C@@H](O)CN2CCC3(CC2)CCN(c2ccc4ncnn4c2)C3=O)ccc2c1COC2=O] | 462 |
| 36 | 23 | 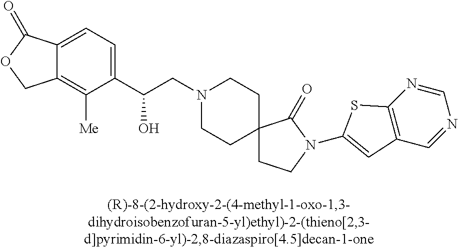 [SMILES: Cc1c([C@@H](O)CN2CCC3(CC2)CCN(c2cc4cncnc4s2)C3=O)ccc2c1COC2=O] | 479 |
| 37 | 23 | 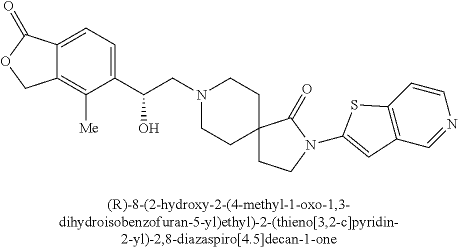 [SMILES: Cc1c([C@@H](O)CN2CCC3(CC2)CCN(c2cc4cnccc4s2)C3=O)ccc2c1COC2=O] | 478 |

### Example 38

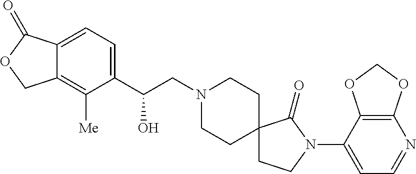 [SMILES: Cc1c([C@@H](O)CN2CCC3(CC2)CCN(c2ccnc4c2OCO4)C3=O)ccc2c1COC2=O]

Potassium phosphate (111 mg, 0.523 mmol), trans-(1R,2R)-N,N′-bismethyl-1,2-cyclohexanediamine (8.24 μL, 0.052 mmol), 7-bromo-[1,3]dioxolo[4,5-b]pyridine (35 mg, 0.174 mmol), (R)-8-(2-hydroxy-2-(4-methyl-1-oxo-1,3-dihydroisobenzofuran-5-yl)ethyl)-2,8-diazaspiro[4.5]decan-1-one (INTERMEDIATE 23) (60 mg, 0.174 mmol), and copper(I) iodide (9.95 mg, 0.052 mmol) were charged to a microwave vial. The vial was sealed, degassed, and filled with dioxane (871 μL). The reaction mixture was heated at 110° C. over two days, and was diluted with EtOAc and DCM, then filtered through CELITE®. The filtrate was concentrated and the crude product was purified by column chromatography (0-10% MeOH/DCM) to afford (R)-2-([1,3]dioxolo[4,5-b]pyridin-7-yl)-8-(2-hydroxy-2-(4-methyl-1-oxo-1,3-dihydroisobenzofuran-5-yl)ethyl)-2,8-diazaspiro[4.5]decan-1-one. LC/MS: [(M+1)]+=466

### Example 39

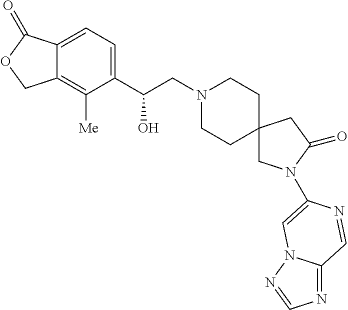 [SMILES: Cc1c([C@@H](O)CN2CCC3(CC2)CC(=O)N(c2cn4ncnc4cn2)C3)ccc2c1COC2=O]

2-([1,2,4]Triazolo[1,5-a]pyrazin-6-yl)-2,8-diazaspiro[4.5]decan-3-one (INTERMEDIATE 50) (61 mg, 0.197 mmol) was dissolved in 2 mL of ethanol in a 5 mL microwave vial. Polymer supported carbonate (3 eq) was then added, followed by addition of 4-methyl-5-[(2R)-oxiran-2-yl]-2-benzofuran-1(3H)-one (INTERMEDIATE 1B) (67 mg, 0.352 mmol). The vial is capped and the mixture was irradiated at 140° C. for 55 min. The polymer resin was then filtered off. The crude mixture was purified by reverse phase mass directed HPLC system using acetonitrile/water as the mobile phase to give (R)-2-([1,2,4]triazolo[1,5-a]pyrazin-6-yl)-8-(2-hydroxy-2-(4-methyl-1-oxo-1,3-dihydroisobenzofuran-5-yl)ethyl)-2,8-diazaspiro[4.5]decan-3-one. LC-MS (IE, m/z): 463.45 (M+1)⁺.

The compounds in Table 3 were prepared in an analogous fashion to that described for Example 39. The column having the heading INT provides the numbers that represent each of the two intermediates that were combined to make each exemplified compound.

TABLE 3

| | | | LC/MS |
|---|---|---|---|
| EX. | INT. | EXAMPLE STRUCTURE/NAME | (M + 1)^{+:} |
| 40 | 52, 1B | 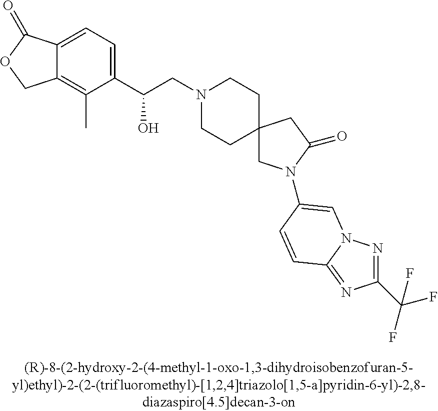 [SMILES: Cc1c([C@@H](O)CN2CCC3(CC2)CC(=O)N(c2ccc4nc(C(F)(F)F)nn4c2)C3)ccc2c1COC2=O] | 530 |
| 41 | 51, 1B | 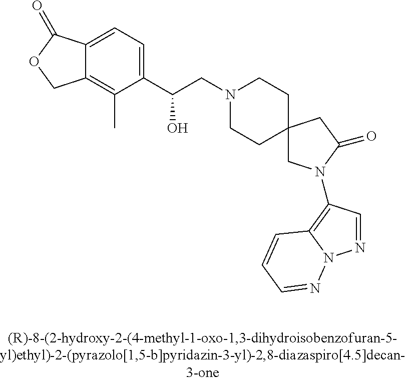 [SMILES: Cc1c([C@@H](O)CN2CCC3(CC2)CC(=O)N(c2cnn4ncccc24)C3)ccc2c1COC2=O] | 462 |
| 42 | 25, 2A | 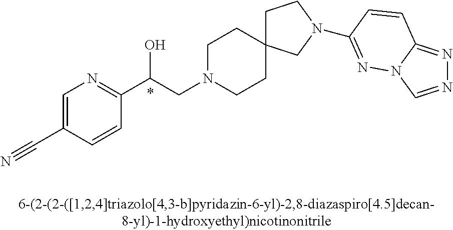 [SMILES: N#Cc1ccc(C(O)CN2CCC3(CC2)CCN(c2ccc4nncn4n2)C3)nc1] | 405 |
| 43 | 25, 2B | 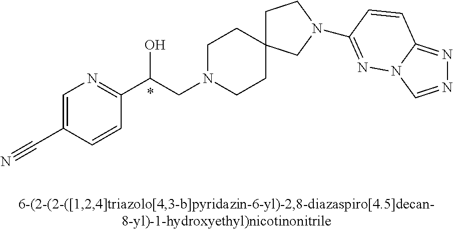 [SMILES: N#Cc1ccc(C(O)CN2CCC3(CC2)CCN(c2ccc4nncn4n2)C3)nc1] | 405 |
| 44 | 25, 6A | 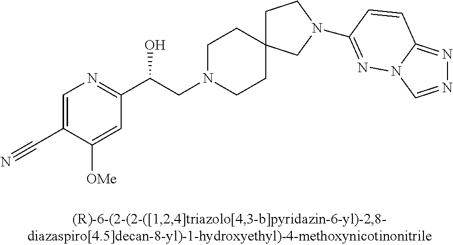 [SMILES: COc1cc([C@H](O)CN2CCC3(CC2)CCN(c2ccc4nncn4n2)C3)ncc1C#N] | 435 |
| 45 | 25, 6B | 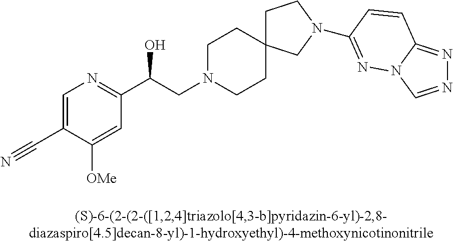 [SMILES: COc1cc([C@@H](O)CN2CCC3(CC2)CCN(c2ccc4nncn4n2)C3)ncc1C#N] | 435 |
| 46 | 25, 5A | 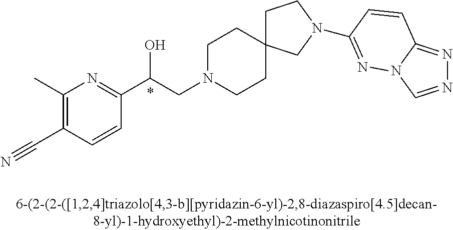 [SMILES: Cc1nc(C(O)CN2CCC3(CC2)CCN(c2ccc4nncn4n2)C3)ccc1C#N] | 419 |
| 47 | 25, 5B | 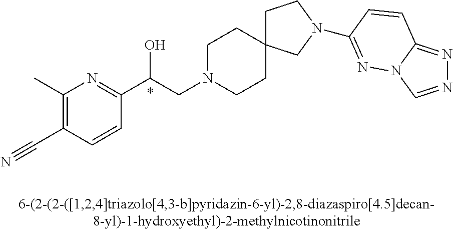 [SMILES: Cc1nc(C(O)CN2CCC3(CC2)CCN(c2ccc4nncn4n2)C3)ccc1C#N] | 419 |
| 48 | 25, 4A | 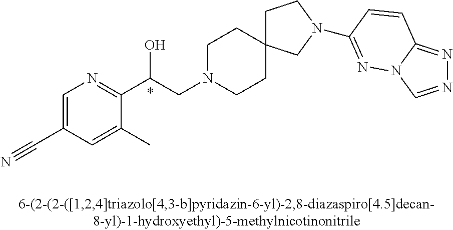 [SMILES: Cc1cc(C#N)cnc1C(O)CN1CCC2(CC1)CCN(c1ccc3nncn3n1)C2] | 419 |
| 49 | 25, 4B | 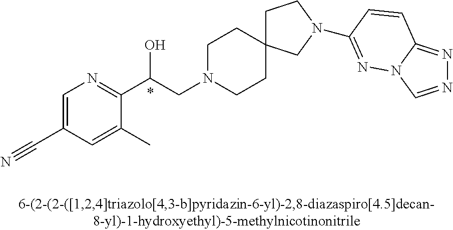 [SMILES: Cc1cc(C#N)cnc1C(O)CN1CCC2(CC1)CCN(c1ccc3nncn3n1)C2] | 419 |
| 50 | 25, 7A | 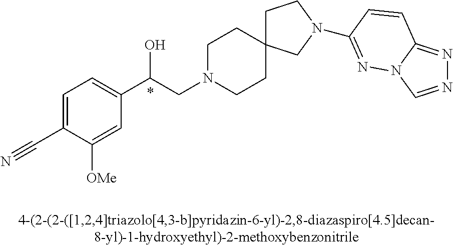 [SMILES: COc1cc(C(O)CN2CCC3(CC2)CCN(c2ccc4nncn4n2)C3)ccc1C#N] | 434 |
| 51 | 25, 7B | 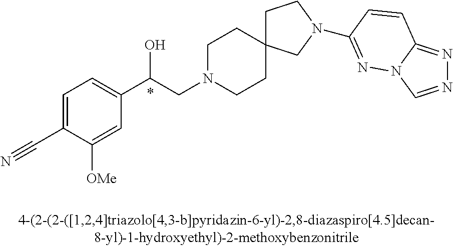 [SMILES: COc1cc(C(O)CN2CCC3(CC2)CCN(c2ccc4nncn4n2)C3)ccc1C#N] | 434 |
| 52 | 25, 12 (racemic) | 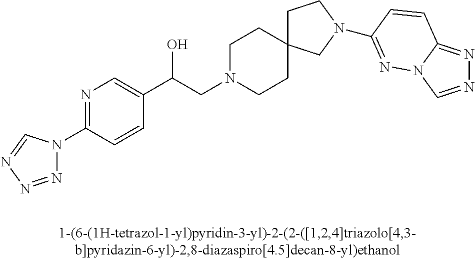 [SMILES: OC(CN1CCC2(CC1)CCN(c1ccc3nncn3n1)C2)c1ccc(-n2cnnn2)nc1] | 448 |
| 53 | 25, 9 (racemic) | 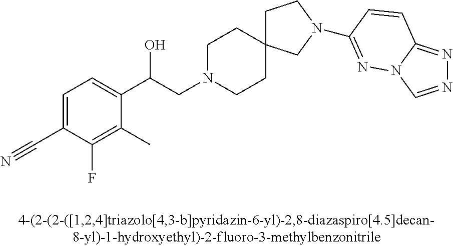 [SMILES: Cc1c(C(O)CN2CCC3(CC2)CCN(c2ccc4nncn4n2)C3)ccc(C#N)c1F] | 436 |
| 54 | 25, 10 (racemic) | 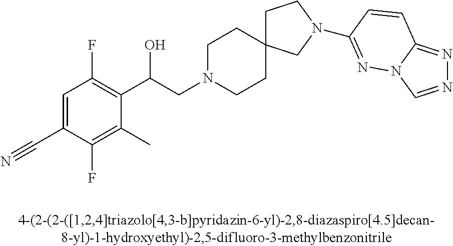 [SMILES: Cc1c(F)c(C#N)cc(F)c1C(O)CN1CCC2(CC1)CCN(c1ccc3nncn3n1)C2] | 454 |
| 55 | 25, 11 (racemic) | 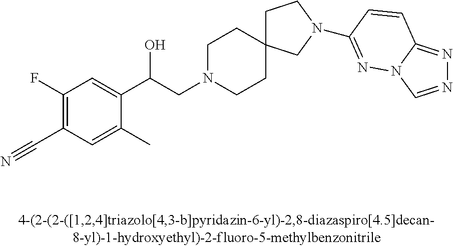 [SMILES: Cc1cc(C#N)c(F)cc1C(O)CN1CCC2(CC1)CCN(c1ccc3nncn3n1)C2] | 436 |
| 56 | 25, 8 | 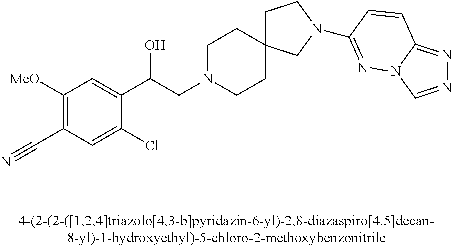 [SMILES: COc1cc(C(O)CN2CCC3(CC2)CCN(c2ccc4nncn4n2)C3)c(Cl)cc1C#N] | 468 |
| 57 | 53, 5A | 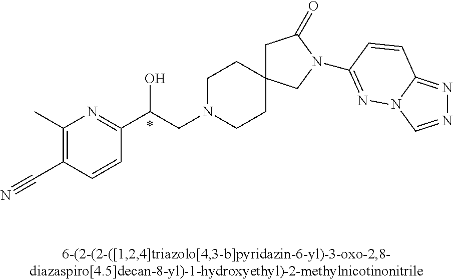 [SMILES: Cc1nc(C(O)CN2CCC3(CC2)CC(=O)N(c2ccc4nncn4n2)C3)ccc1C#N] | 433 |
| 58 | 53, 7B | 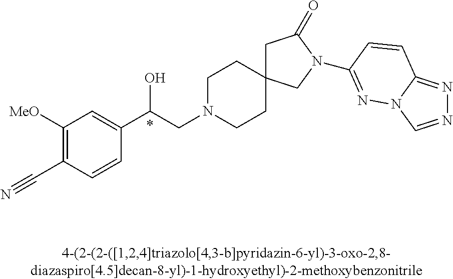 [SMILES: COc1cc(C(O)CN2CCC3(CC2)CC(=O)N(c2ccc4nncn4n2)C3)ccc1C#N] | 448 |
| 59 | 53, 2A | 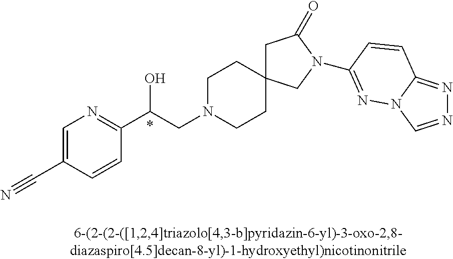 [SMILES: N#Cc1ccc(C(O)CN2CCC3(CC2)CC(=O)N(c2ccc4nncn4n2)C3)nc1] | 419 |
| 60 | 53, 6A | 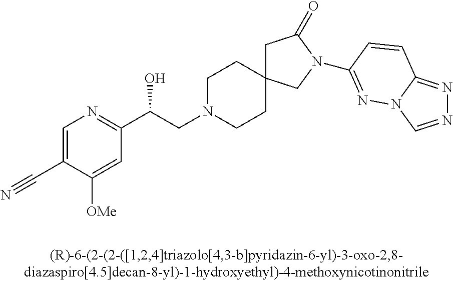 [SMILES: COc1cc([C@H](O)CN2CCC3(CC2)CC(=O)N(c2ccc4nncn4n2)C3)ncc1C#N] | 449 |
| 61 | 53, 6B | 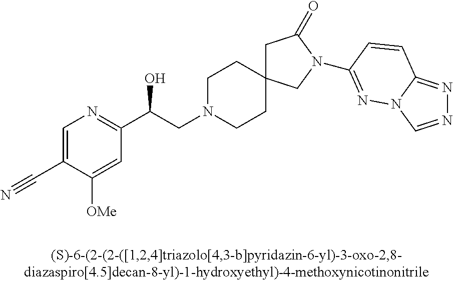 [SMILES: COc1cc([C@@H](O)CN2CCC3(CC2)CC(=O)N(c2ccc4nncn4n2)C3)ncc1C#N] | 449 |
| 62 | 54, 2A | 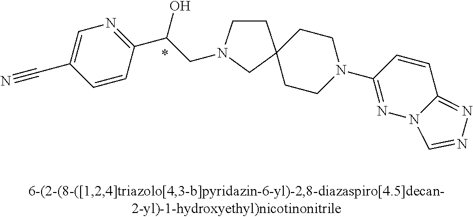 [SMILES: N#Cc1ccc(C(O)CN2CCC3(CCN(c4ccc5nncn5n4)CC3)C2)nc1] | 405 |
| 63 | 54, 2B | 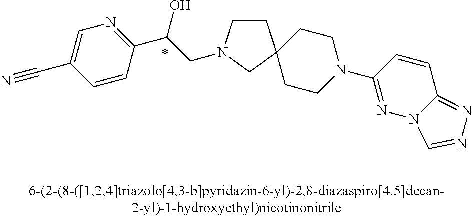 [SMILES: N#Cc1ccc(C(O)CN2CCC3(CCN(c4ccc5nncn5n4)CC3)C2)nc1] | 405 |
| 64 | 54, 6A | 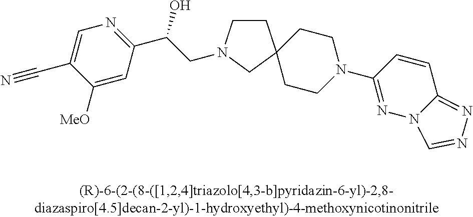 [SMILES: COc1cc([C@H](O)CN2CCC3(CCN(c4ccc5nncn5n4)CC3)C2)ncc1C#N] | 435 |
| 65 | 54, 4A | 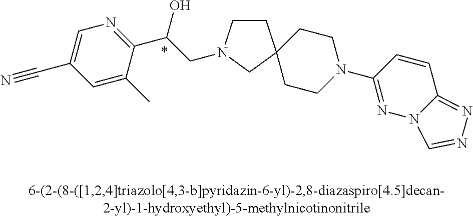 [SMILES: Cc1cc(C#N)cnc1C(O)CN1CCC2(CCN(c3ccc4nncn4n3)CC2)C1] | 419 |
| 66 | 54, 4B | 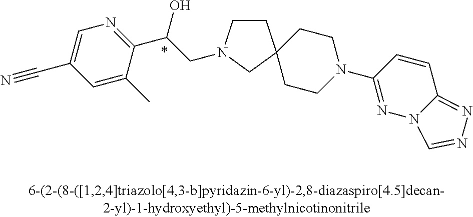 [SMILES: Cc1cc(C#N)cnc1C(O)CN1CCC2(CCN(c3ccc4nncn4n3)CC2)C1] | 419 |
| 67 | 54, 5A | 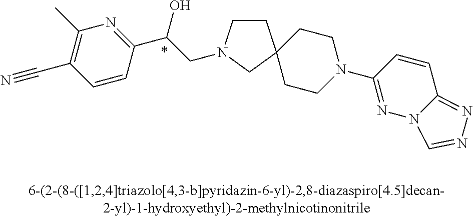 [SMILES: Cc1nc(C(O)CN2CCC3(CCN(c4ccc5nncn5n4)CC3)C2)ccc1C#N] | 419 |
| 68 | 54, 5B | 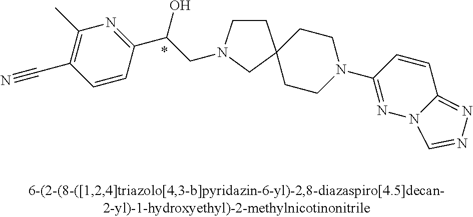 [SMILES: Cc1nc(C(O)CN2CCC3(CCN(c4ccc5nncn5n4)CC3)C2)ccc1C#N] | 419 |
| 69 | 54, 12 (racemic) | 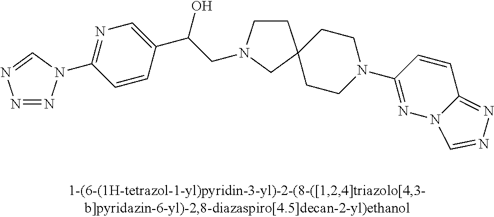 [SMILES: OC(CN1CCC2(CCN(c3ccc4nncn4n3)CC2)C1)c1ccc(-n2cnnn2)nc1] | 448 |
| 70 | 54, 1B | 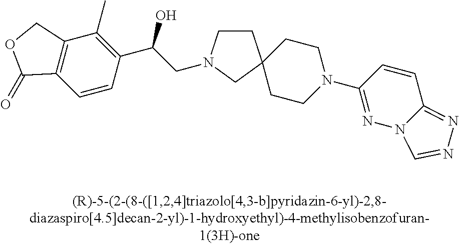 [SMILES: Cc1c([C@@H](O)CN2CCC3(CCN(c4ccc5nncn5n4)CC3)C2)ccc2c1COC2=O] | 449 |

### Example 71

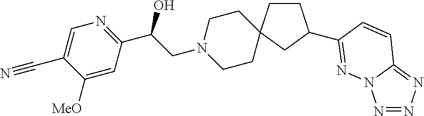 [SMILES: COc1cc([C@@H](O)CN2CCC3(CCC(c4ccc5nnnn5n4)C3)CC2)ncc1C#N]

A microwave tube was charged with 6-(2,8-diazaspiro[4.5]decan-2-yl)tetrazolo[1,5-b]pyridazine (INTERMEDIATE 26) (0.025 g, 0.096 mmol), 4-methoxy-6-(oxiran-2-yl)nicotinonitrile (INTERMEDIATE 6, isomer B) (0.017 g, 0.096 mmol) and ethanol (1.0 mL). The solution was degassed and filled with nitrogen (3×), then sealed and heated in a microwave reactor to 140° C. for 1 hour. The reaction was cooled to room temperature and concentrated in vacuo. The resulting residue was purified by prep TLC (5% MeOH:DCM) to provide (S)-6-(1-hydroxy-2-(2-(tetrazolo[1,5-b]pyridazin-6-yl)-2,8-diazaspiro[4.5]decan-8-yl)ethyl)-4-methoxynicotinonitrile. ¹H NMR (500 MHz, CDCl₃): δ 8.60 (s, 1H), 8.03 (m, 1H), 7.32 (m, 1H), 7.01 (s, 1H), 4.83 (m, 1H), 4.03 (s, 3H), 3.72 (m, 2H), 3.56 (m, 2H), 2.98 (m, 2H), 2.94 (m, 1H), 2.78 (m, 1H), 2.59 (m, 2H), 2.48 (m, 2H), 1.81 (m, 4H); LC-MS (IE, m/z): 436 [M+1]⁺.

The compounds in Table 4 were synthesized in analogous fashion to that described for EXAMPLE 71. The column having the heading INT provides the numbers that represent each of the two intermediates that were combined to make each exemplified compound.

TABLE 4

| | | | LC/MS |
|---|---|---|---|
| EX | INT | EXAMPLE STRUCTURE/NAME | (M + 1)^{+:} |
| 72 | 28, 6B | 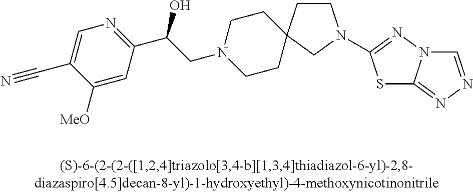 [SMILES: COc1cc([C@@H](O)CN2CCC3(CC2)CCN(c2nn4cnnc4s2)C3)ncc1C#N] | 441 |
| 73 | 29, 6B | 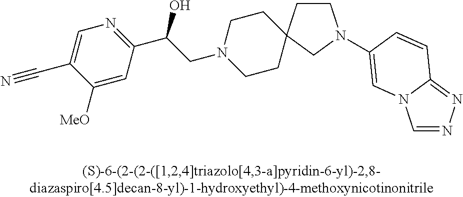 [SMILES: COc1cc([C@@H](O)CN2CCC3(CC2)CCN(c2ccc4nncn4c2)C3)ncc1C#N] | 434 |
| 74 | 30 1B | 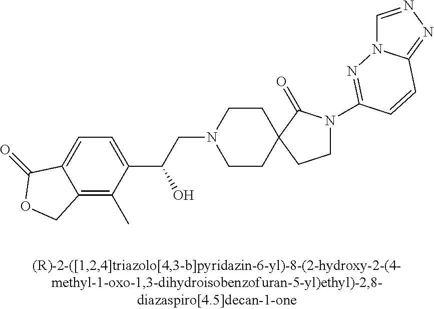 [SMILES: Cc1c([C@@H](O)CN2CCC3(CC2)CCN(c2ccc4nncn4n2)C3=O)ccc2c1COC2=O] | 463 |
| 75 | 30, 6B | 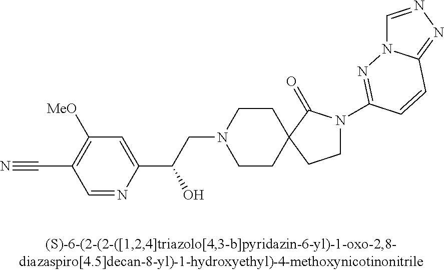 [SMILES: COc1cc([C@@H](O)CN2CCC3(CC2)CCN(c2ccc4nncn4n2)C3=O)ncc1C#N] | 449 |
| 76 | 41, 1B | 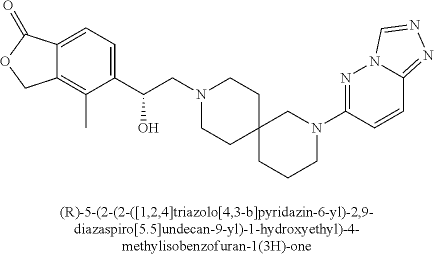 [SMILES: Cc1c([C@@H](O)CN2CCC3(CCCN(c4ccc5nncn5n4)C3)CC2)ccc2c1COC2=O] | 463 |
| 77 | 42, 6A | 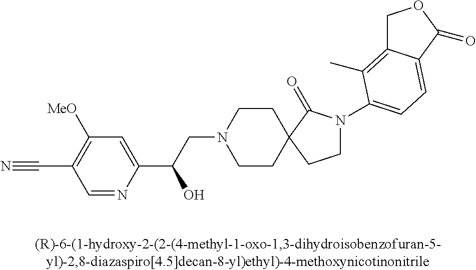 [SMILES: COc1cc([C@H](O)CN2CCC3(CC2)CCN(c2ccc4c(c2C)COC4=O)C3=O)ncc1C#N] | 463 |
| 78 | 42, 57A | 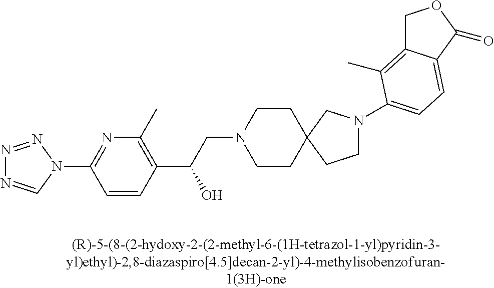 [SMILES: Cc1nc(-n2cnnn2)ccc1[C@@H](O)CN1CCC2(CC1)CCN(c1ccc3c(c1C)COC3=O)C2] | 490 |
| 79 | 42, 1B | 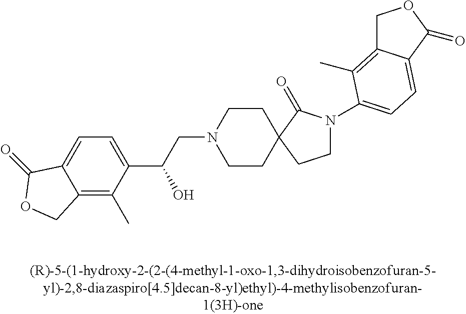 [SMILES: Cc1c([C@@H](O)CN2CCC3(CC2)CCN(c2ccc4c(c2C)COC4=O)C3=O)ccc2c1COC2=O] | 477 |
| 80 | 44, 1B | 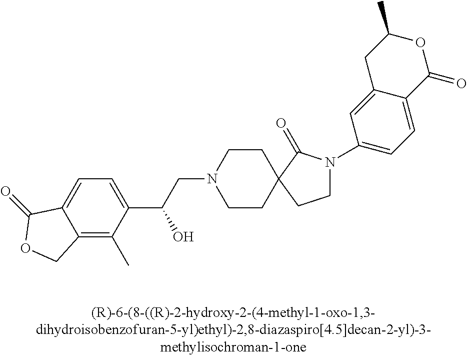 [SMILES: Cc1c([C@@H](O)CN2CCC3(CC2)CCN(c2ccc4c(c2)C[C@@H](C)OC4=O)C3=O)ccc2c1COC2=O] | 491 |
| 81 | 31, 1B | 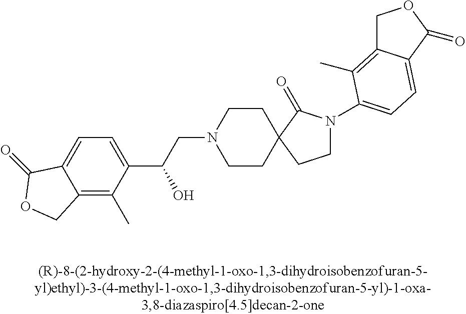 [SMILES: Cc1c([C@@H](O)CN2CCC3(CC2)CCN(c2ccc4c(c2C)COC4=O)C3=O)ccc2c1COC2=O] | 493 |
| 82 | 32, 1B | 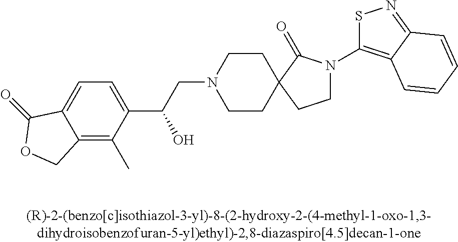 [SMILES: Cc1c([C@@H](O)CN2CCC3(CC2)CCN(c2snc4ccccc24)C3=O)ccc2c1COC2=O] | 478 |
| 83 | 33, 1B | 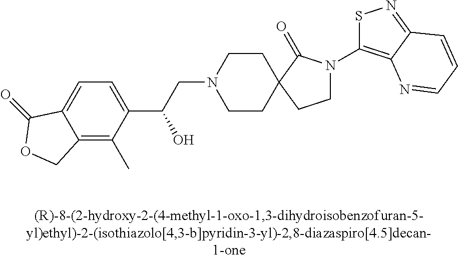 [SMILES: Cc1c([C@@H](O)CN2CCC3(CC2)CCN(c2snc4cccnc24)C3=O)ccc2c1COC2=O] | 479 |
| 84 | 34, 1B | 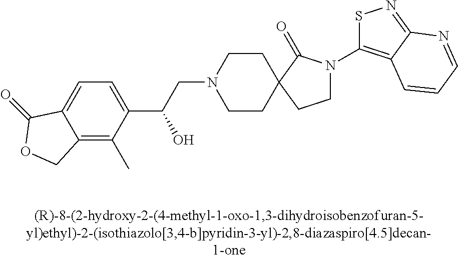 [SMILES: Cc1c([C@@H](O)CN2CCC3(CC2)CCN(c2snc4ncccc24)C3=O)ccc2c1COC2=O] | 479 |
| 85 | 36, 1B | 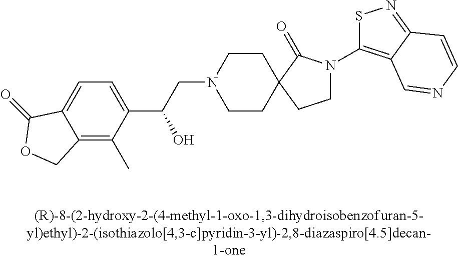 [SMILES: Cc1c([C@@H](O)CN2CCC3(CC2)CCN(c2snc4ccncc24)C3=O)ccc2c1COC2=O] | 479 |
| 86 | 37, 1B | 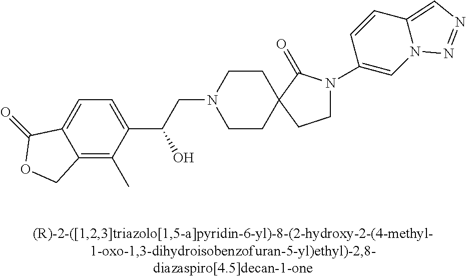 [SMILES: Cc1c([C@@H](O)CN2CCC3(CC2)CCN(c2ccc4cnnn4c2)C3=O)ccc2c1COC2=O] | 462 |
| 87 | 39, 1B | 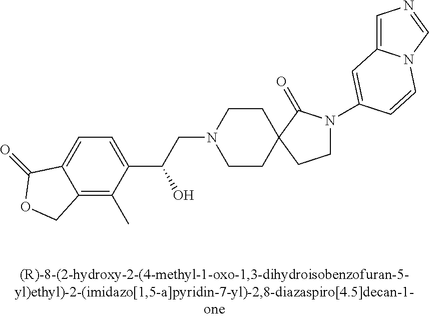 [SMILES: Cc1c([C@@H](O)CN2CCC3(CC2)CCN(c2ccn4cncc4c2)C3=O)ccc2c1COC2=O] | 461 |
| 88 | 40, 1B | 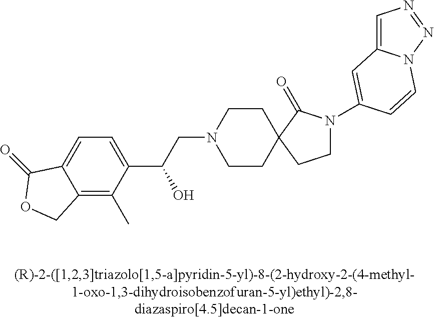 [SMILES: Cc1c([C@@H](O)CN2CCC3(CC2)CCN(c2ccn4nncc4c2)C3=O)ccc2c1COC2=O] | 462 |
| 89 | 47, 1B | 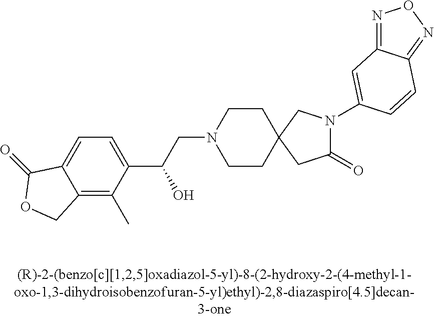 [SMILES: Cc1c([C@@H](O)CN2CCC3(CC2)CC(=O)N(c2ccc4nonc4c2)C3)ccc2c1COC2=O] | 463 |
| 90 | 45, 1B | 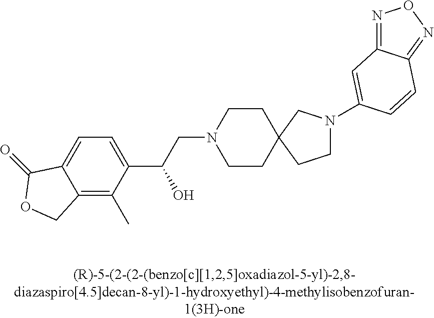 [SMILES: Cc1c([C@@H](O)CN2CCC3(CC2)CCN(c2ccc4nonc4c2)C3)ccc2c1COC2=O] | 449 |
| 91 | 55, 1B | 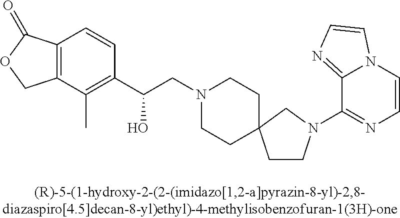 [SMILES: Cc1c([C@@H](O)CN2CCC3(CC2)CCN(c2nccn4ccnc24)C3)ccc2c1COC2=O] | 448 |
| 92 | 46, 1B | 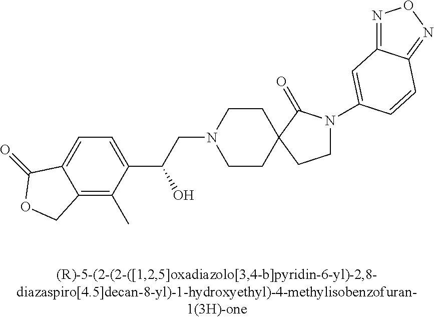 [SMILES: Cc1c([C@@H](O)CN2CCC3(CC2)CCN(c2ccc4nonc4c2)C3=O)ccc2c1COC2=O] | 450 |
| 93 | 48, 1B | 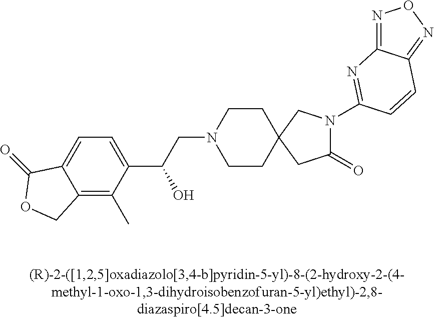 [SMILES: Cc1c([C@@H](O)CN2CCC3(CC2)CC(=O)N(c2ccc4nonc4n2)C3)ccc2c1COC2=O] | 464 |
| 94 | 49, 1B | 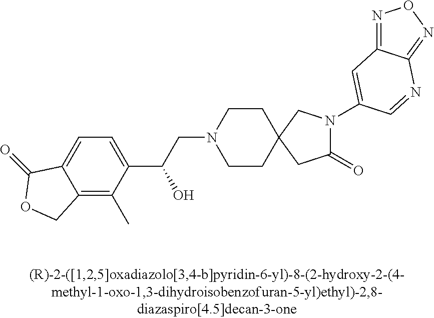 [SMILES: Cc1c([C@@H](O)CN2CCC3(CC2)CC(=O)N(c2cnc4nonc4c2)C3)ccc2c1COC2=O] | 464 |
| 95 | 58, 1B | 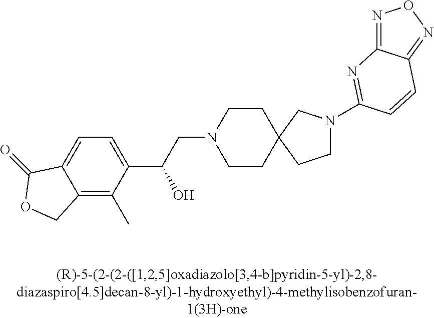 [SMILES: Cc1c([C@@H](O)CN2CCC3(CC2)CCN(c2ccc4nonc4n2)C3)ccc2c1COC2=O] | 450 |

The following Thallium Flux Assay was performed on each of the final product compounds in the Examples.

### Thallium Flux Assay

### Cell Culture Conditions—

HEK293 cells stably expressing hROMK (hKᵢᵣ1.1) were grown at 37° C. in a 10% CO₂ humidified incubator in complete growth media: Dulbecco's Modified Eagle Medium supplemented with non-essential amino acids, Penicillin/Streptomycin/Glutamine, G418 and FBS. At >80% confluency, the media was aspirated from the flask and rinsed with 10 mL calcium/magnesium-free phosphate buffered saline (PBS). 5 mL of 1× trypsin (prepared in Ca/Mg Free PBS) was added to T-225 flask and the flask was returned to 37° C./CO₂ incubator for 2-3 minutes. To dislodge the cells, the side of the flask was gently banged with one's hand. The cells were completely titrated and then the cells were transferred to 25 mL complete media, centrifuged at 1,500 rpm for 6 min followed by resuspension in complete growth media, and the cell concentration was determined. For typical re-seeding, 4E6 cells/T-225 flask will attain >80% confluency in 4 days. Under ideal growth conditions and appropriate tissue culture practices, this cell line is stable for 40-45 passages.

### FluxOR Kit Components (Invitrogen F10017)

FluxOR™ Reagent (Component A)

FluxOR™ Assay Buffer (Component B)—10× Concentrate

PowerLoad™ Concentrate (Component C)—100× Concentrate

Probenecid (Component D)—Lyophilized sample is kept at −20° C. Water soluble, 100× after solubilization in 1 mL water. Store at 4° C.

FluxOR™ Chloride-free Buffer (Component E)—5× Concentrate

Potassium sulfate (K₂SO₄) Concentrate (Component F)—125 mM in water. Store at 4° C.

Thallium sulfate (Tl₂SO₄) Concentrate (Component G)—50 mM in water. Store at 4° C.

DMSO (dimethyl sulfoxide, Component H)—1 mL (100%)

### Reagent Preparation: FluxOR Working Solutions

1000× FluxOR™ Reagent: Reconstitute a vial of component A in 100 μl DMSO; Mix well; Store 10 μl aliquots at −20° C.

1× FluxOR™ Assay Buffer: Dilute Component B 10-fold with water; Adjust pH to 7.4 with Hepes/NaOH; Filter and store at 4° C.

Probenecid/Assay Buffer: 100 mL of 1× FluxOR™ Assay Buffer; 1 mL of reconstituted component D; Store at 4° C.

Loading Buffer (per microplate): 10 μl 1000× FluxOR™ Reagent; 100 μl component C; 10 mL Probenecid/Assay Buffer

Compound Buffer (per microplate): 20 mL Probenecid/Assay Buffer; 0.3 mM ouabain (10 mM ouabain in water can be stored in amber bottle/aluminum foil at room temperature); Test compound

1× FluxOR™Chloride-Free Buffer: Prepare 1× working solution in water. Can be stored at room temperature

Stimulant Buffer (prepared at 5× final concentration in 1× FluxOR™Chloride-Free Buffer): 7.5 mM thallium sulfate and 0.75 mM potassium sulfate (to give a final assay concentration of 3 mM Thallium/0.3 mM potassium). Store at 4° C. when not in use. If kept sterile, this solution is good for months.

### Assay Protocol—

The ROMK channel functional thallium flux assay was performed in 384 wells, using the FLIPR-Tetra instrument. HEK-hKir1.1 cells were seeded in Poly-D-Lysine microplates and kept in a 37° C.-10% CO₂ incubator overnight. On the day of the experiment, the growth media was replaced with the FluxOR™ reagent loading buffer and incubated, protected from light, at ambient temperature (23-25° C.) for 90 min. The loading buffer was replaced with assay buffer±test compound followed by 30 min incubation at ambient temperature, where the thallium/potassium stimulant was added to the microplate.

### Step Protocol

1. Seed HEK-hKir1.1 cells (50 μl at 20,000 cells/well) in 384-well PDL coated Microplates
2. Allow cells to adhere overnight in humidified 37° C./10% CO₂ incubator
3. Completely remove cell growth media from microplate and replace with 25 μl loading buffer
4. Incubate Microplate at room temperature, protected form light, for 90 min
5. Remove loading buffer and replace with 25 μl 1× Assay Buffer±test compound.
6. Incubate microplate at room temperature, protected from light, for 30 min
7. At FLIPR-Tetra 384: Add stimulant (thallium/potassium) solution to microplate and monitor fluorescence. Excitation=400 nm, Emission=460 & 580 nm. Collect data for ˜10 min.

### Data Calculation—

The fluorescence intensity of wells containing 3 μM of a standard control ROMK inhibitor of the present invention was used to define the ROMK-sensitive component of thallium flux. Fluorescence in the presence of test compounds was normalized to control values to provide % fluorescence change. IC₅₀ values represent the concentration of compound that inhibited 50% of the ROMK thallium flux signal.

### Assay Standard—

Normally, a control compound is included to support that the assay is giving consistent results compared to previous measurements, although the control is not required to obtain the results for the test compounds. The control can be any compound of Formula I of the present invention, preferably with an IC₅₀ potency of less than 1 μM in this assay. Alternatively, the control could be another compound (outside the scope of Formula I) that has an IC₅₀ potency in this assay of less than 1 μM.

Data collected for compounds in the Examples of the present invention using the Thallium Flux Assay are shown in Table 5 below. All of the tested final product compounds in the Examples (diastereomeric mixtures and individual diastereomers) had IC₅₀ potencies less than 1 μM as determined by the Thallium Flux Assay.

TABLE 5

| Example Number | IC50 (μM) | Example Number | IC50 (μM) |
|---|---|---|---|
| 1 | 0.7455 | 2 | 0.1317 |
| 3 | 0.2354 | 4 | 0.7789 |
| 5 | 0.07472 | 6 | 0.3038 |
| 7 | 0.6204 | 8 | 0.2463 |
| 9 | 0.2612 | 10 | 0.01278 |
| 11 | 0.04769 | 12 | 0.3062 |
| 13 | 0.4111 | 14 | 0.204 |
| 15 | 0.2867 | 16 | 0.0978 |
| 17 | 0.4857 | 18 | 0.3394 |
| 19 | 0.07469 | 20 | 0.5134 |
| 21 | 0.236 | 22 | 0.2117 |
| 23 | 0.1083 | 24 | 0.85 |
| 25 | 0.3424 | 26 | 0.1993 |
| 27 | 0.3174 | 28 | 0.2612 |
| 29 | 0.301 | 30 | 0.08321 |
| 31 | 0.2672 | 32 | 0.04882 |
| 33 | 0.2843 | 34 | 0.4269 |
| 35 | 0.5368 | 36 | 0.1896 |
| 37 | 0.1173 | 38 | 0.05492 |
| 39 | 0.4583 | 40 | 0.8942 |
| 41 | 0.3363 | 42 | 0.1174 |
| 43 | 0.3606 | 44 | 0.1237 |
| 45 | 0.0459 | 46 | 0.07696 |
| 47 | 0.1474 | 48 | 0.08814 |
| 49 | 0.2076 | 50 | 0.04148 |
| 51 | 0.0487 | 52 | 0.08395 |
| 53 | 0.01872 | 54 | 0.03591 |
| 55 | 0.026 | 56 | 0.02763 |
| 57 | 0.248 | 58 | 0.4126 |
| 59 | 0.4123 | 60 | 0.7797 |
| 61 | 0.2283 | 62 | 0.1293 |
| 63 | 0.1545 | 64 | 0.09184 |
| 65 | 0.03471 | 66 | 0.07283 |
| 67 | 0.06418 | 68 | 0.08588 |
| 69 | 0.07258 | 70 | 0.03098 |
| 71 | 0.2077 | 72 | 0.2504 |
| 73 | 0.2209 | 74 | 0.116 |
| 75 | 0.6079 | 76 | 0.0976 |
| 77 | 0.06638 | 78 | 0.6222 |
| 79 | 0.019 | 80 | 0.093 |
| 81 | 0.0914 | 82 | 0.03261 |
| 83 | 0.03386 | 84 | 0.0571 |
| 85 | 0.01852 | 86 | 0.1717 |
| 87 | 0.2597 | 88 | 0.02923 |
| 89 | 0.04275 | 90 | 0.09828 |
| 91 | 0.6991 | 92 | 0.02732 |
| 93 | 0.2736 | 94 | 0.1086 |
| 95 | 0.01916 | | |

While the invention has been described with reference to certain particular embodiments thereof, numerous alternative embodiments will be apparent to those skilled in the art from the teachings described herein. The scope of the claims should not be limited by the preferred embodiments set forth in the examples, but should be given the broadest interpretation consistent with the description as a whole. Recitation or depiction of a specific compound in the claims (i.e., a species) without a specific stereoconfiguration designation, or with such a designation for less than all chiral centers, is intended to encompass the racemate, racemic mixtures, each individual enantiomer, a diastereoisomeric mixture and each individual diastereomer of the compound where such forms are possible due to the presence of one or more asymmetric centers. All patents, patent applications and publications cited herein are incorporated by reference in their entirety.

1. A compound of formula I: 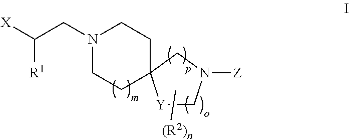
or a pharmaceutically acceptable salt thereof,
wherein:
X is 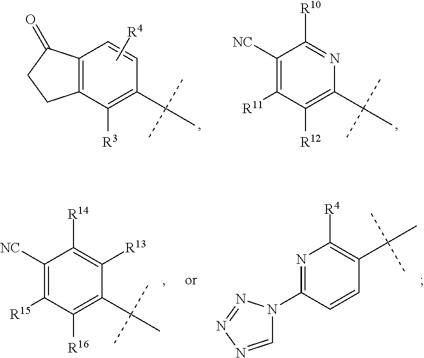
Y is —O— or —CH₂—;
Z is a N-containing multicyclic heteroaromatic group, which is optionally substituted with one R⁶ group, or is a group of the formula: 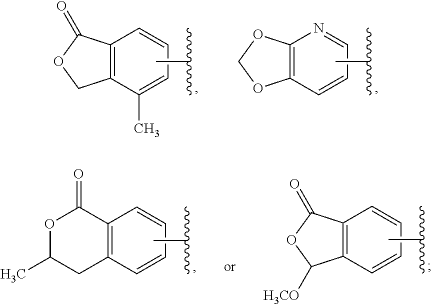
R is H, C₁₋₂ alkyl optionally substituted with 1-3 halogens, or —C(O)R⁵;
R¹ is —OR or halogen;
R² is oxo or C₁₋₂ alkyl optionally substituted with 1-3 F;
R³ is H or CH₃;
R⁴ is H or CH₃;
R⁵ is CH₃ or C₃₋₆cycloalkyl;
R⁶ is halogen, —CN, C₃₋₆ cycloalkyl, furanyl, —SO₂N(R⁸)(R⁹), C₁₋₂ alkyl which is optionally substituted with —SR⁷ or 1-5 halogens, or —OC₁₋₂ alkyl which is optionally substituted with 1-5 halogens;
R⁷ is allyl or C₁₋₂ alkyl;
R⁸ is H or CH₃;
R⁹ is H or CH₃;
R¹⁰ is H, C₁₋₂ alkyl, or —OCH₃;
R¹¹ is H, C₁₋₂ alkyl, or —OCH₃;
R¹² is H, C₁₋₂ alkyl or —OCH₃;
R¹³ is H, halogen, C₁₋₂ alkyl or —OCH₃;
R¹⁴ is H, halogen, C₁₋₂ alkyl or —OCH₃;
R¹⁵ is H, halogen, C₁₋₂ alkyl or —OCH₃;
R¹⁶ is H, halogen, C₁₋₂ alkyl or —OCH₃;
m is 0 or 1;
n is 0 or 1;
o is 0, 1 or 2; and
p is 1, 2, or 3;
provided that o+p=2 or 3.

2. The compound as defined in claim 1 wherein the N-containing multicyclic heteroaromatic group is

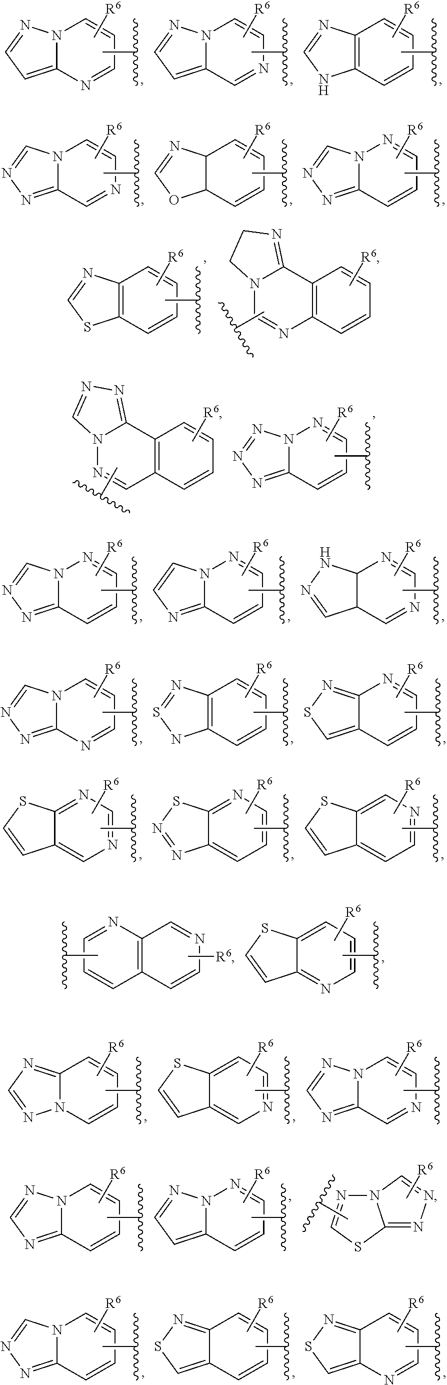

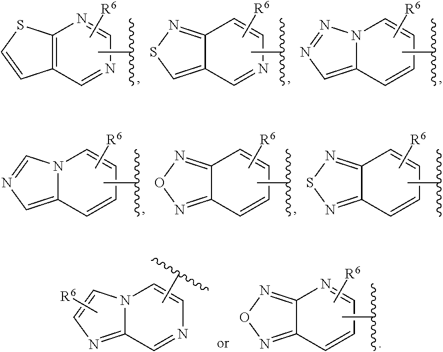

3. The compound as defined in claim 1, which has the formula II: 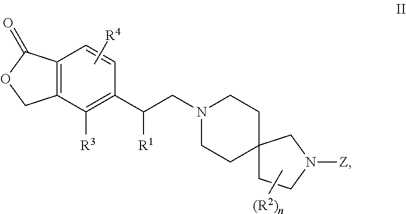
or a pharmaceutically acceptable salt thereof.

4. The compound as defined in claim 1, which has the formula III: 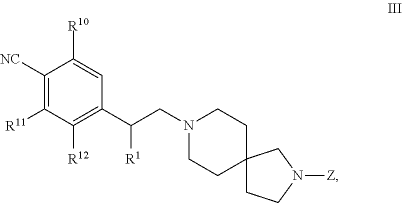
or a pharmaceutically acceptable salt thereof,
wherein:
Z is

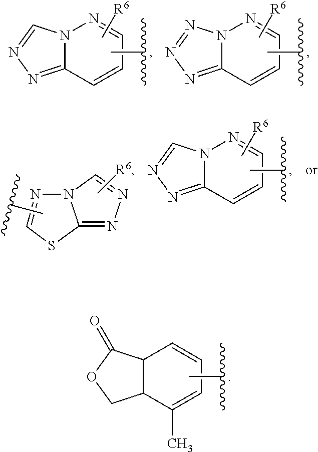

5. The compound as defined claim 1, which has the formula IV or IVa: 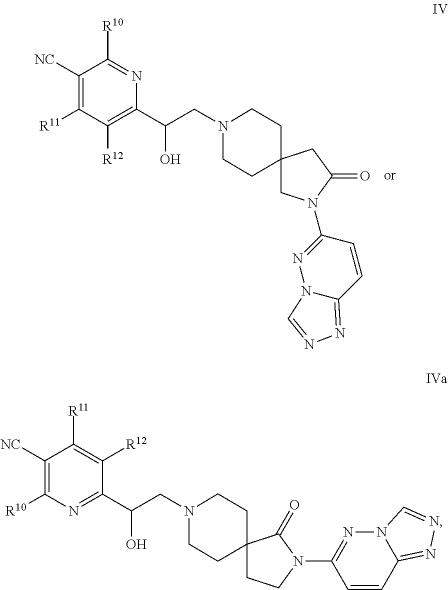
or a pharmaceutically acceptable salt thereof.

6. The compound as defined in claim 1, which has the formula V: 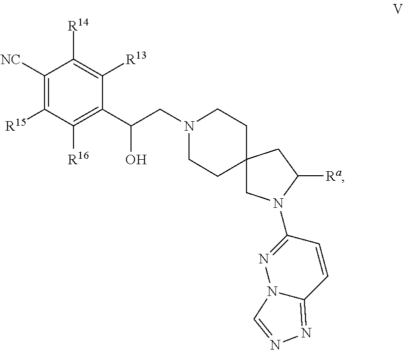
or a pharmaceutically acceptable salt thereof,
wherein:
R^{a} is H or oxo;
R¹³ is H, halogen, C₁₋₂ alkyl, or —OC₁₋₂ alkyl;
R¹⁴ is H, halogen, C₁₋₂ alkyl, or —OC₁₋₂ alkyl;
R¹⁵ is H, halogen, C₁₋₂ alkyl, or —OC₁₋₂ alkyl, and
R¹⁶ is H, halogen, C₁₋₂ alkyl or —OC₁₋₂ alkyl.

7. The compound as defined in claim 1 having the formula VI: 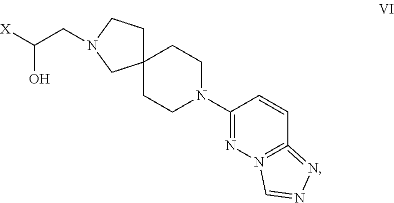
or a pharmaceutically acceptable salt thereof,
wherein:
X is 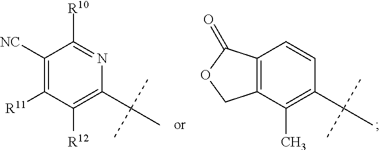
R¹⁰ is H or C₁₋₂ alkyl;
R¹¹ is H, C₁₋₂ alkyl, or —OC₁₋₂ alkyl; and
R¹² is H, C₁₋₂ alkyl, or —OC₁₋₂ alkyl.

8. The compound as defined in claim 1, which has formula VII or VIII: 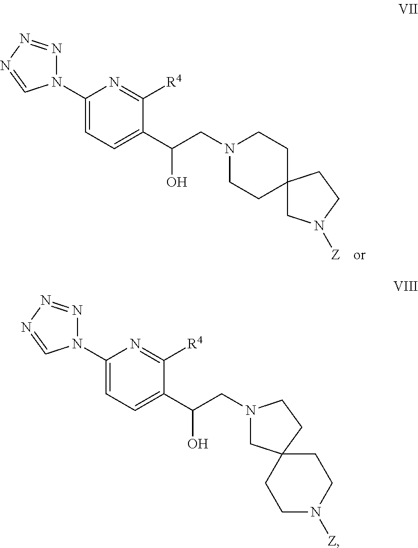
or a pharmaceutically acceptable salt thereof,
wherein:
Z is

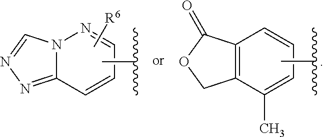

9. The compound as defined in claim 1, which has the formula IX: 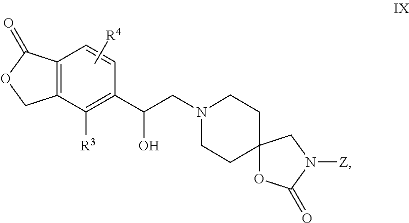
or a pharmaceutically acceptable salt thereof,
wherein
Z is

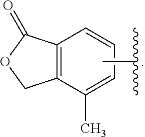

10. The compound as defined in claim 1, which has the formula X or XI: 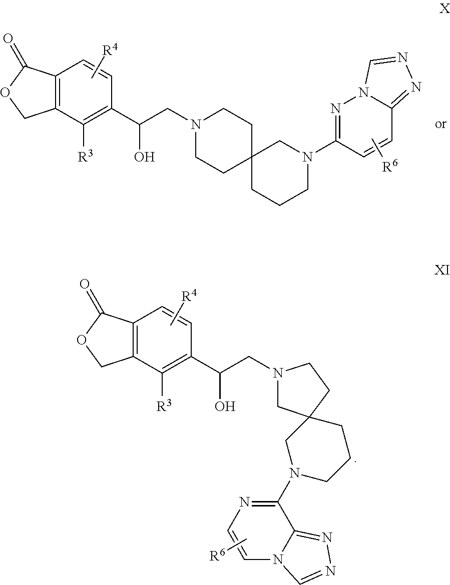
or a pharmaceutically acceptable salt thereof.

11. The compound as defined in claim 3, which has the formula IIa: 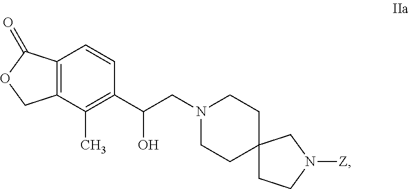
or a pharmaceutically acceptable salt thereof,
wherein:
Z is:

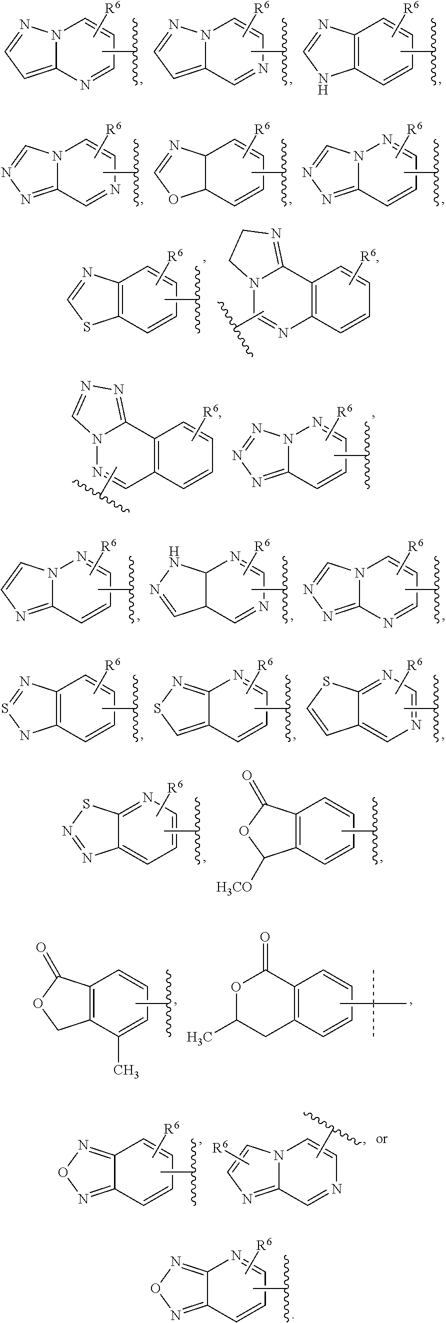

12. The compound as defined in claim 3, which has the formula IIb: 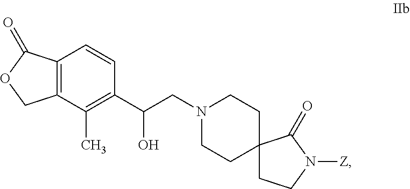
or a pharmaceutically acceptable salt thereof,
wherein:
Z is

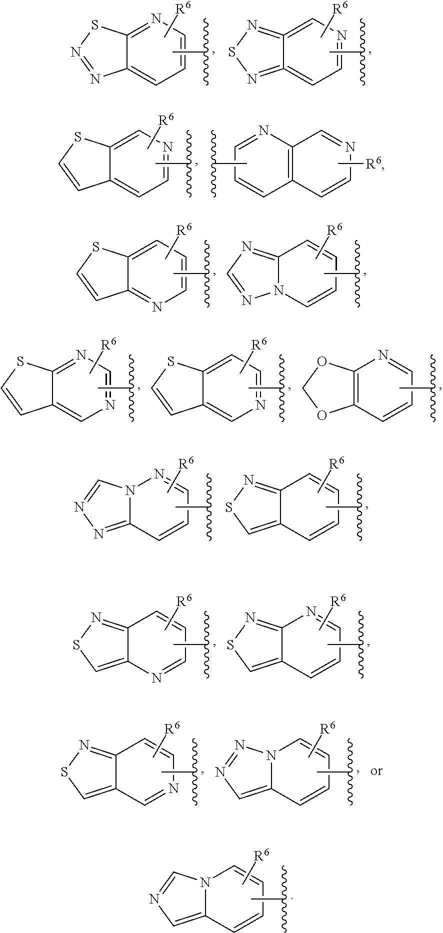

13. The compound as defined in claim 1, which has the formula IIc: 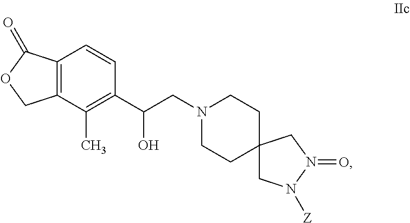
or a pharmaceutically acceptable salt thereof,
wherein:
Z is

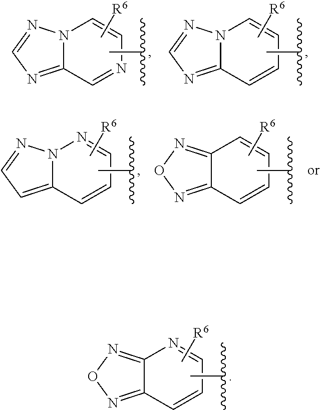

14. A compound as defined in claim 1, which is:
(R)-5-(2-(2-([1,2,4]triazolo[4,3-b]pyridazin-6-yl)-2,8-diazaspiro[4.5]decan-8-yl)-1-hydroxyethyl)-4-methylisobenzofuran-1(3H)-one; (Ex. 5)
(R)-5-(1-hydroxy-2-(2-(tetrazolo[1,5-b]pyridazin-6-yl)-2,8-diazaspiro[4.5]decan-8-yl)ethyl)-4-methylisobenzofuran-1(3H)-one; (Ex 10);
(R)-2-([1,3]dioxolo[4,5-b]pyridin-7-yl)-8-(2-hydroxy-2-(4-methyl-1-oxo-1,3-dihydroisobenzofuran-5-yl)ethyl)-2,8-diazaspiro[4.5]decan-1-one; (Ex 38)
6-(2-(2-([1,2,4]triazolo[4,3-b]pyridazin-6-yl)-2,8-diazaspiro[4.5]decan-8-yl)-1-hydroxyethyl)-2-methylnicotinonitrile; (Ex 46)
4-(2-(2-([1,2,4]triazolo[4,3-b]pyridazin-6-yl)-2,8-diazaspiro[4.5]decan-8-yl)-1-hydroxyethyl)-2,5-difluoro-3-methylbenzonitrile; (54)
6-(2-(8-([1,2,4]triazolo[4,3-b]pyridazin-6-yl)-2,8-diazaspiro[4.5]decan-2-yl)-1-hydroxyethyl)-5-methylnicotinonitrile; (Ex 65)
6-(2-(8-([1,2,4]triazolo[4,3-b]pyridazin-6-yl)-2,8-diazaspiro[4.5]decan-2-yl)-1-hydroxyethyl)-2-methylnicotinonitrile; (Ex 67)
(R)-5-(2-(8-([1,2,4]triazolo[4,3-b]pyridazin-6-yl)-2,8-diazaspiro[4.5]decan-2-yl)-1-hydroxyethyl)-4-methylisobenzofuran-1(3H)-one; (Ex 70)
(R)-6-(1-hydroxy-2-(2-(4-methyl-1-oxo-1,3-dihydroisobenzofuran-5-yl)-2,8-diazaspiro[4.5]decan-8-yl)ethyl)-4-methoxynicotinonitrile; (Ex. 77)
(R)-5-(1-hydroxy-2-(2-(4-methyl-1-oxo-1,3-dihydroisobenzofuran-5-yl)-2,8-diazaspiro[4.5]decan-8-yl)ethyl)-4-methylisobenzofuran-1(3H)-one; (Ex 79)
(R)-8-(2-hydroxy-2-(4-methyl-1-oxo-1,3-dihydroisobenzofuran-5-yl)ethyl)-3-(4-methyl-1-oxo-1,3-dihydroisobenzofuran-5-yl)-1-oxa-3,8-diazaspiro[4.5]decan-2-one; (Ex. 81)
(R)-2-([1,2,3]triazolo[1,5-a]pyridin-5-yl)-8-(2-hydroxy-2-(4-methyl-1-oxo-1,3-dihydroisobenzofuran-5-yl)ethyl)-2,8-diazaspiro[4.5]decan-1-one; (Ex 88)
(R)-2-(benzo[c][1,2,5]oxadiazol-5-yl)-8-(2-hydroxy-2-(4-methyl-1-oxo-1,3-dihydroisobenzofuran-5-yl)ethyl)-2,8-diazaspiro[4.5]decan-3-one; (Ex 89);
(R)-5-(2-(2-([1,2,5]oxadiazolo[3,4-b]pyridin-6-yl)-2,8-diazaspiro[4.5]decan-8-yl)-1-hydroxyethyl)-4-methylisobenzofuran-1(3H)-one; (Ex 92)
(R)-5-(2-(2-([1,2,5]oxadiazolo[3,4-b]pyridin-5-yl)-2,8-diazaspiro[4.5]decan-8-yl)-1-hydroxyethyl)-4-methylisobenzofuran-1(3H)-one; (Ex 95)
or a pharmaceutically acceptable salt thereof.

15. A pharmaceutical composition comprising a therapeutically effective amount of a compound as defined in claim 1 or a pharmaceutically acceptable salt thereof and a pharmaceutically acceptable carrier.

16. The pharmaceutical composition as defined in claim 15, which further comprises a therapeutically effective amount of at least one additional therapeutic agent.

17. The pharmaceutical composition as defined in claim 16, wherein the additional therapeutic agent is losartan, valsartan, candesartan, olmesartan, telmesartan, eprosartan, irbesartan, amlodipine, alacepril, benazepril, captopril, ceronapril, cilazapril, delapril, enalapril, enalaprilat, fosinopril, imidapril, lisinopril, moveltipril, perindopril, quinapril, ramipril, spirapril, temocapril, or trandolapril, amiloride, spironolactone, epleranone or triamterene, or a pro-drug thereof, or a pharmaceutically acceptable salt of any of the foregoing

18. A method for inhibiting ROMK comprising administering to a patient in need thereof a therapeutically effective amount of the compound defined in claim 1 or a pharmaceutically acceptable salt thereof.

19. A method for causing natriuresis comprising administering to a patient in need thereof a therapeutically effective amount of the compound defined in claim 1 or a pharmaceutically acceptable salt thereof.

20. A method for the treatment of one or more disorders selected from hypertension, acute heart failure, chronic heart failure, pulmonary arterial hypertension, cardiovascular disease, diabetes, endothelial dysfunction, diastolic dysfunction, stable and unstable angina pectoris, thromboses, restenosis, myocardial infarction, stroke, cardiac insufficiency, pulmonary hypertonia, atherosclerosis, hepatic cirrhosis, ascitis, pre-eclampsia, cerebral edema, nephropathy, nephrotic syndrome, acute kidney insufficiency, chronic kidney disease, hypercalcemia, Dent's disease, Meniere's disease, or edematous states in a patient in need thereof comprising administering an effective amount of a compound as defined in claim 1 or a pharmaceutically acceptable salt thereof to said patient.

21. (canceled)

22. (canceled)
